(19) United States
(12) Patent Application Publication
(10) Pub. No.: US 2026/0076616 A1
(43) Pub. Date: Mar. 19, 2026

(54) CENTRALIZED HUB DEVICE FOR DETERMINING AND DISPLAYING HEALTH-RELATED METRICS

(71) Applicant: Sleep Number Corporation, Minneapolis, MN (US)

(72) Inventors: Faisal Mushtaq, San Diego, CA (US); Stacy Stusynski, Blaine, MN (US); Cory Lee Grabinger, Maple Grove, MN (US); Caleb Siffring, Victoria, MN (US); Gary N. Garcia Molina, Madison, WI (US)

(21) Appl. No.: 19/332,370

(22) Filed: Sep. 18, 2025

Related U.S. Application Data
(63) Continuation of application No. 17/573,175, filed on Jan. 11, 2022.

(51) Int. Cl. A61B 5/00; A61B 5/0205; A61B 5/021; A61B 5/024; A61B 5/08; A61B 5/083; A61B 5/1455; A61M 21/00; G16H 40/67

(52) U.S. Cl. CPC A61B 5/4815; A61B 5/0205; A61B 5/0816; A61B 5/0836; A61B 5/14551; A61B 5/6898; A61B 5/7275; A61B 5/742; A61B 5/746; A61B 5/747; A61B 5/7475; A61M 21/00; G16H 40/67; A61B 5/021; A61B 5/02405; A61B 2560/0242; A61M 2021/0027

(57) ABSTRACT

Described are systems for beds that can include sensors for sensing physical phenomena in an environment surrounding a bed, a display for outputting information about the environment, the bed, and a sleeper, and a controller communicably coupled to the sensors. The controller can receive the sensed physical phenomena from the sensors, analyze the physical phenomena to determine at least one of environmental, sleep, and health metrics of a sleeper in the bed, and determine, based on at least one of the environmental, sleep, and health metrics of the sleeper, control signals to modify the environment surrounding the bed. The controller can also output, at the display, the environmental, sleep, and health metrics of the sleeper. The controller can also transmit the control signals to a second controller in order to engage a home automation device. The physical phenomena can include ambient sound, ambient light, ambient CO2 concentration, and/or ambient temperature.

### CROSS REFERENCE TO RELATED APPLICATIONS

This application is a continuation of U.S. application Ser. No. 17/573,175, filed Jan. 11, 2022, the contents of which is hereby incorporated by reference.

### TECHNICAL FIELD

This document relates to a centralized device for determining sleep-related insights and controlling components in a sleep environment, such as an air bed and home automation devices.

### BACKGROUND

In general, a bed is a piece of furniture used as a location to sleep or relax. Many modern beds include a soft mattress on a bed frame. The mattress may include springs, foam material, and/or an air chamber to support the weight of one or more occupants.

### SUMMARY

This document generally relates to a centralized hub device that can detect physical phenomena in an environment and determine environmental, health, and sleep metrics based on that physical phenomena. More particularly, this document provides for systems and methods to monitor, evaluate, and modify a sleep environment to promote health, detect adverse conditions, and recommend actionable strategies to improve the environment.

The disclosed technology can provide for determining user health information (e.g., apnea, cardiovascular health, fever, predicting health issues, sleep walking analysis, snore analysis, etc.), determining improved sleep settings (e.g., environment score, improving sleep quality, sleep insight, smart home integration, etc.), determining and engaging bed controls (e.g., closed-loop, environment score, greater automation, improving sleep quality, etc.), smart home automation and connectivity (e.g., improving sleep quality, lighting, mirroring settings, improved settings for sleep, etc.), visualization of metrics associated with the user, additional user interaction with their sleep environment (e.g., digital dream diary, pressure signal as an environment sensor, voice control, etc.), determining an environment score (e.g., improving sleep quality, making a sleep score more meaningful and insightful, quantification, etc.), monitoring the user's sleep (e.g., accuracy of sleep monitoring, posture, etc.), clustering users into cohorts to glean additional sleep and health data about a particular user (e.g., user-facing interface), improved storage of information and data, and privacy protection.

The hub device can be an integrated nightstand device having sensors and a user interface, such as a display, to track and output environmental, health, and sleep metrics to a user of a bed in the environment. The sensors can detect a plurality of different types of physical phenomena. The hub device can process such physical phenomena to determine whether the environment is providing a user with conditions that facilitate improved sleep quality. Such conditions can include, but are not limited to, lighting, sound, temperature, humidity, and CO₂ concentration in the environment. The hub device can also process the sensed/detected physical phenomena to determine health-related information about the user and perform sleep quality analysis for the user.

The user interface of the hub device can output information to the user and receive user input. The user interface can be a touchscreen display that outputs information such as the determined environmental, health, and sleep metrics. The user interface can also output third party services and applications that are accessible via a connection (e.g., wired and/or wireless) and/or downloaded at the hub device. The user can therefore interact with third party services and applications by using the hub device. The hub device can receive touch-based user input and/or audio user input for navigating the user interface, interacting with the third party services and applications, and controlling one or more components in the environment (e.g., such as an adjustable foundation, an air mattress, an HVAC system, and/or home automation devices).

The hub device can be utilized for home automation. For example, the hub device can automatically adjust settings or components in the environment to desired or automatically calculated settings that improve the user's sleep quality. The hub device can also transmit control signals to one or more controllers that engage home automation devices in the environment. Moreover, the user can manually control settings and/or components in the environment via user input at the hub device.

The hub device can also quantify risk associated with health and/or sleep metrics that are determined for the user. Based on this risk quantification, the hub device can determine whether healthcare providers should be notified of the user's current condition. The hub device can transmit notifications or alerts to one or more different healthcare providers based on identifying that the user is experiencing and/or developing health-related issues. The hub device can therefore provide early warning and detection of health-related issues for bed users to healthcare providers.

In some implementations, the disclosed technology can provide for determining a health score (e.g., between 0 and 100) that corresponds to improved or preferred health of the particular user. The health score can quantify cardiovascular health and can be used to screen the user for chronic conditions, such as apnea. If, for example, the health score is below a certain threshold level, the hub device can recommend measures that can be taken with regards to other parameters (e.g., blood pressure, SpO₂, etc.) in order to improve the user's health. The hub device may identify an influence of environmental factors on the health score and suggest actionable ways to increase the health score by modifying the environment. The hub device can automatically implement the suggestions using connection with home automation devices and/or smart integration.

Similarly, the disclosed technology provides for determining improved or preferred settings for sleep. An ambient score (e.g., between 0 and 100) can be determined and can correspond to improved, restful sleep. The hub device can learn from the environment when the user's sleep is disturbed. The hub device can accordingly coach the user to prepare the environment so that they can experience improved sleep through each stage of the user's sleep cycle.

Some embodiments described herein include a system having sensors that can sense physical phenomena in an environment surrounding a bed, a display that can output information about the environment, the bed, and a sleeper in the bed, and a controller communicably coupled to the plurality of sensors. The controller can receive the sensed physical phenomena from the sensors, analyze the physical phenomena to determine at least one of environmental, sleep, and health metrics of a sleeper in the bed, and determine, based on at least one of the environmental, sleep, and health metrics of the sleeper, one or more control signals to modify the environment surrounding the bed.

Embodiments described herein can include one or more optional features. For example, the controller can output, at the display, at least one of the environmental, sleep, and health metrics of the sleeper. The controller can also transmit the one or more control signals to a second controller in order to engage a home automation device.

In some implementations, the physical phenomena can include at least one of ambient sound, ambient light, ambient CO₂ concentration, and ambient temperature. The ambient CO₂ concentration can be greater than a threshold level and the one or more control signals can include ventilating the environment surrounding the bed until a desired CO₂ concentration is detected by one or more of the plurality of sensors. The desired CO₂ concentration can be less than 800 parts per million (ppm). The ambient sound can be greater than a threshold level and the one or more control signals can include maintaining sound exposure during a sleep cycle of the sleeper at a desired sound level as detected by one or more of the plurality of sensors. The desired sound level can be less than 30 decibels (dB). The ambient temperature can be greater than a threshold level and the one or more control signals can include lowering a temperature of the environment until a desired temperature is reached and detected by one or more of the plurality of sensors. The desired temperature can be greater than or equal to 60 degrees Fahrenheit and less than or equal to 70 degrees Fahrenheit. The ambient light can be greater than a threshold level and the one or more control signals can include maintaining illumination in the environment at a desired illumination level as detected by one or more of the plurality of sensors. The desired illumination level can be less than 10 lux (lx).

In some implementations, one or more of the sensors can include audio, light, CO₂ concentration, temperature, humidity, motion, volatile organic compounds, electromagnetic interference, atmospheric pressure, systolic blood pressure (SBP), oxygen saturation (SPO₂), pulse, heartrate (HR), and radar sensors.

As another example, the physical phenomena can include at least one of a heartrate variability (HRV), HR, respiratory rate (RR), SPO₂, SBP, and diastolic blood pressure (DBP), and the controller can analyze the physical phenomena to determine health metrics of a sleeper in the bed using at least one of age, gender, and body mass index (BMI) of the sleeper. The controller can also determine whether the determined health metrics of the sleeper are within predetermined value ranges for each of the health metrics of the sleeper. The controller may quantify a risk level associated with the one or more health metrics based on a determination that the one or more health metrics are not within predetermined value ranges, generate an alert based on the quantified risk level, and output the alert at the display. Sometimes, the controller can also transmit an alert about the one or more health metrics to a medical provider based on a determination that the risk level associated with the one or more health metrics exceeds a threshold risk level.

In some implementations, one or more of the sensors can be communicably coupled to the bed and can sense physical phenomena on the bed. The controller can be separate from the sensors, the display, and the bed, and the controller can be a cloud based system. Sometimes, the audio sensor can detect audio at 20 KHz, and the controller can determine, based on the detected audio, information about the sleeper's sleep cycle and sleep quality. The information about the sleeper's sleep cycle and sleep quality can include snore and sleep apnea.

As another example, the controller can determine at least one of environmental, sleep, and health metrics of a sleeper in the bed further based on at least one of (i) sleep quality information that is provided as user input at the display and (ii) physical phenomena that are sensed by one or more wearable devices and external sensors in communication with the controller. The light sensor can detect illumination values every 5 minutes, and the controller can determine, based on the detected illumination values, changes in HR of the sleeper. The CO₂ concentration sensor can detect CO₂ concentration levels every 5 minutes, and the controller can determine, based on the detected CO₂ concentration levels, sleep fragmentation of the sleeper. The temperature sensor can detect temperature of the environment every 5 minutes, and the controller can determine, based on the detected temperature, how long it takes the sleeper to fall asleep and how long the sleeper experiences restful sleep. The SPO₂ sensor can detect spot measurements from an optical signal acquired at a sampling rate in a range of 0.1 Hz to 1 KHz, and the controller can determine, based on the spot measurements, blood pressure of the sleeper. The HR sensor can detect spot measurements from at least one of an optical signal and a galvanic signal acquired at a sampling rate in a range of 0.1 Hz to 1 KHz, and the controller can determine, based on the spot measurements, heartrate and blood pressure of the sleeper. The motion sensor can detect motion every 10 seconds, and the controller can determine, based on the detected motion, whether the sleeper sleepwalks or experiences REM sleep disorders. The sensors can include an external sensor that cam detect blood pressure readings of the sleeper, and the controller can determine, based on the detected blood pressure readings, information about the sleeper's sleep cycle and sleep quality.

In some implementations, the display can be a touchscreen that is integrated into the system, and the system can be positioned proximate to the bed in the environment. The controller can also execute the one or more control signals, the control signals including adjusting pressure settings of the bed, raising one or more portions of the bed, lowering one or more portions of the bed, activating a heating or cooling element of the bed, activating a night light, and activating an alarm clock.

The display can output data about the sleeper's sleep quality and sleep cycle. The display can also receive audio input from the sleeper to control one or more home automation devices. The display can also display user-selected pictures. The display can also output a graphical user interface (GUI) that includes selectable options for the sleeper to interact with third party mobile applications that are downloaded to or accessible via the display. The display can output a health dashboard for the sleeper, weather data, stock quotes, security information, lighting information, and HVAC information. The system can also include an external power source that can provide power to at least one of the plurality of sensors, the display, and the controller. The system can also include a speaker that can generate audio output that greets the sleeper when the sleeper wakes up and informs the sleeper of their sleep score.

The devices, system, and techniques described herein may provide one or more of the following advantages. For example, the disclosed technology provides a centralized interface for determining metrics associated with a user's sleep quality, outputting such information to the user, and providing controls to the user to control components in the user's sleep environment. Thus, one device can perform different functionality and provide a centralized location for the user to interact with components in their environment, home automation devices, third party services and applications, and healthcare providers.

As another example, the disclosed technology provides for non-invasive and unobtrusive detection of physical phenomena in a sleep environment that can impact the user's sleep quality and health metrics. The user may not have to wear sensor devices, such as wearables like a wristband, chest strap, or head-worn sensor. Instead, sensors can be integrated into the hub device and configured to sense physical phenomena without touching or interfering with the user's activity. As such, the user does not need to remember to wear, turn on, or interact with the health hub in order for the health hub to work. By comparison, a system requiring activation every night can be forgotten. A wearable, even if it is comfortable enough to sleep with, can still run out of batteries in the middle of the night. The hub device can also communicate with other sensors that are part of the environment and/or integrated into the user's bed. The other sensors can include virtual sensors, or sensors that exist in local infrastructure in the environment and/or that are in communication with the hub device via a network. Thus, the sensors can be external to the environment and can be configured to collect local weather data, which can then be transmitted to the hub device. The sensors can also be configured to detect discrete physical phenomena that other sensors may not be able to detect, such as SpO₂ concentrations that may be influenced by the environment and/or CO₂ concentrations in the environment, and/or that systems such as the hub device may not correlate with sleep quality and overall health of the particular user in that physical environment.

As yet another example, the disclosed technology provides for automatic determination of changes that can be made to the environment to improve the user's sleep quality and/or overall health. Environmental intervention can enhance an aggregate metric of sleep quality, which can be an aggregate of cardiorespiratory metrics along with sleep architecture metrics. As described herein, the hub device can determine changes to make based on detected physical phenomena and data associated with the user's historic sleep data, historic health data, currently detected sleep data, and currently detected health data. The hub device can make minute changes to the environment that otherwise may not be monitored or analyzed in relation to user sleep quality and health metrics by existing systems. In many cases, the changes may be so small as to be imperceptible to the user in a way that disrupts sleep if the user is sleeping or, if the user is away, do not call the user's attention away from their current activities and to the changes. For example, the hub device can monitor and adjust environmental conditions such as illumination, noise, temperature, humidity, CO₂ concentration, and/or VOC concentration, including very small changes (e.g., a light can be dimmed in very small increments, a fan sped up or slowed down). Therefore, the disclosed technology can provide for more accurate determinations of how environmental conditions impact user sleep quality and health metrics and can change the environment to help the user even when the user is not thinking about it.

Similarly, history and context of environmental conditions that lead to better sleep and dependencies thereof can be used to improve sleep quality. Sleep quality (which can be quantified as described herein by aggregating various physiological markers) can be improved through modification of environmental conditions where the modifications can be informed by history, context, and the user's longitudinal data. In other words, combinations of environmental conditions and contextual information (e.g., season, day of the week, latitude/longitude, time-of-day, etc.) that favor better sleep can be stored and used to improve environmental conditions in the user's sleep environment. Historic data, such as temporal changes, is also relevant because some environmental conditions that favor better sleep for young adults may be different compared to older adults. Environmental conditions that favor improved sleep quality, therefore, is not confined to a small region and/or demographic, but rather can be disjointed. Such historic environmental conditions can therefore be used to personalize the user's sleep environment.

The disclosed technology can also provide for automatic risk quantification to determine whether user is experiencing health issues that should be reported out. By monitoring physical phenomena during the user's sleep cycle, the hub device can more accurately track how the user's health conditions trend throughout the sleep cycle. The hub device can determine whether the user's health metrics trend outside of expected ranges for their age, gender, and other user-related information. Based on such continuous, non-invasive monitoring, the hub device can detect health-related issues early enough to get healthcare providers involved. This can have large improvements in health outcomes where early interventions are key for preventing rapid degradation (e.g, stroke or cardiac event). For example, the hub device can determine whether the user is experiencing breathing issues while asleep and whether emergency response personnel should be notified before the user experiences a more serious issue, such as a heart attack. When the hub device identifies abnormal health conditions that may not be as serious or grave as other conditions, the hub device can determine what information should be reported out to healthcare providers who may be assessing what sort of diagnosis or treatment is needed for a particular condition of the user. Thus, the hub device can provide for communication of health-related information with healthcare providers such that the healthcare providers can provide improved and more robust diagnosis and analysis of the user's condition(s).

As described herein, the disclosed technology also provides for improved and more robust analysis about environmental conditions and the user's sleep quality. A variety of physical phenomena can be unobtrusively (e.g., passively) detected and analyzed to determine impacts of such phenomena on the user's sleep quality. The hub device can then determine unobtrusive modifications to make to the environment to improve the user's sleep quality.

The disclosed technology provides for monitoring physical phenomena in such a way that preserves user privacy. Although the hub device may detect noise levels in the environment, the hub device can adjust sound recordings to protect privacy (such as hardware (HW) filtering to prevent aliasing and/or limit delays) while enabling detection of noises that can be used to identify sleep apnea, snoring, and/or disturbing levels of noise that affect sleep quality. A microphone of the hub device can, for example, be restricted to detect certain decibel levels and/or sound wavelengths that do not include sounds corresponding to human speech. As a result, the hub device can passively and/or continuously monitor physical phenomena in the environment to glean more accurate insight into user sleep quality and health while preserving user privacy.

As yet another example, processing can be performed at the hub device to avoid clogging network bandwidth and computing resources. Processing can therefore be performed at the edge, which can be faster and more efficient then analyzing physical phenomena to determine metrics at a remote computing system. The hub device can also communicate with data repositories (e.g., data stores, databases, cloud-based services) to receive population information that can be used to generate additional, personalized insights about preferred conditions in the user's environment.

As mentioned herein, the hub deice can also communicate with other sensors, devices, controllers, and equipment in the environment to more accurately determine metrics and/or conditions associated with the user's sleep quality. Such integration allows for more robust analysis of detected physical phenomena to generate more accurate metrics and suggestions to improve the user's sleep quality.

The details of one or more implementations are set forth in the accompanying drawings and the description below. Other features, aspects and potential advantages will be apparent from the accompanying description and figures.

### DESCRIPTION OF DRAWINGS

FIG. 1 shows an example air bed system.

FIG. 2 is a block diagram of an example of various components of an air bed system.

FIG. 3 shows an example environment including a bed in communication with devices located in and around a home.

FIGS. 4A and 4B are block diagrams of example data processing systems that can be associated with a bed.

FIGS. 5 and 6 are block diagrams of examples of motherboards that can be used in a data processing system associated with a bed.

FIG. 7 is a block diagram of an example of a daughterboard that can be used in a data processing system associated with a bed.

FIG. 8 is a block diagram of an example of a motherboard with no daughterboard that can be used in a data processing system associated with a bed.

FIG. 9 is a block diagram of an example of a sensory array that can be used in a data processing system associated with a bed.

FIG. 10 is a block diagram of an example of a control array that can be used in a data processing system associated with a bed

FIG. 11 is a block diagram of an example of a computing device that can be used in a data processing system associated with a bed.

FIGS. 12-16 are block diagrams of example cloud services that can be used in a data processing system associated with a bed.

FIG. 17 is a block diagram of an example of using a data processing system that can be associated with a bed to automate peripherals around the bed.

FIG. 18 is a schematic diagram that shows an example of a computing device and a mobile computing device.

FIG. 19 is an overview conceptual diagram of an environment having a hub device that performs the techniques described herein.

FIGS. 20A-G depict example graphical user interfaces (GUIs) presented at the hub device.

FIG. 21 is a conceptual diagram for monitoring vital signs of a user and assessing a need to report such vital signs to a healthcare provider.

FIG. 22 is a conceptual diagram for determining changes to make to an environment based on monitoring ambient environmental conditions.

FIG. 23 is a conceptual diagram of combined sensor analysis.

FIG. 24 is a system diagram of components that perform the techniques described herein.

FIGS. 25A-C are system diagrams of components that perform the techniques described herein.

FIGS. 26A-B is a flowchart of a process for modifying an environment based on monitoring physical phenomena in the environment.

FIGS. 27A-B depict example implementations of the hub device described herein.

Like reference symbols in the various drawings indicate like elements.

### DETAILED DESCRIPTION

This document generally describes systems and processes for providing centralized processing of physical phenomena to determine environmental, sleep, and health metrics as well as controls to adjust home automation devices and other components in a physical environment. A hub device can perform the processes described herein. The hub device can also provide a user interface that provides interaction between users and third party services and applications.

The hub device described herein can provide numerous features. The hub device can include a speaker that outputs messages to a nearby user. For example, the hub device can generate output that says good morning to the user and tells the user what their sleep score or other sleep quality information is for a previous sleep cycle. The hub device can be a closed loop system that detects sleep apnea and addresses positional apnea via articulation of one or more components of a bed system. The hub device may alert one or more components in the environment to take heartrate (HR) measurements of a sleeping user when the hub device determines that the user experienced variations in HR throughout a sleep cycle. The hub device can also generate recommendations about measuring blood pressure and/or oxygen saturation (SpO₂). Blood pressure (BP) can fall during sleep, which is important to monitor for cardiovascular health. However, it may not be possible or practice to measure BP during an entire night or sleep cycle. Thus, the hub device can quantify BP dipping probability using BP that is detected at different times throughout the night.

The hub device can also provide for monitoring weight and/or body mass index (BMI) in a seamless manner every day that the user is in the environment. Similarly, the hub device can monitor heart health of the user. The hub device may determine an association between HR decrease during sleep with environmental conditions. For example, HR may not decrease if brightness prior to sleep onset is too high. The hub device can determine how much to adjust brightness, and sometimes automatically make such adjustments, in order to assist the user in experiencing lower HR during sleep.

The hub device can also determine when the user is sick. For example, the hub device can communicate with bed thermistors for fever detection. As another example, the hub device can determine if and when environmental conditions contribute to the user becoming sick. The hub device can predict health issues and/or causes of health-related issues. For example, the hub device can determine that VOC levels in the user's bedroom exceeded some threshold level and therefore contributed to the user falling ill. Any monitored and/or determined health metrics can be shared with not only the user but also medical or other healthcare providers. Such metrics can be automatically transmitted to the medical or other healthcare providers (e.g., when the metrics exceed predetermined risk thresholds). Such metrics can also be transmitted to the providers by user input provided at the hub device.

As additional examples, the hub device can generate health scores for the user (e.g., on a scale of 0-100) that corresponds to improved or preferred health and that is based on one or more detected physical phenomena in the environment. The hub device can communicate with motion sensors to detect episodes of somnambulism and/or REM behavior disorder(s). Similarly, the hub device can detect and identify instances of sleep walking. The hub device can determine an influence that CO₂ concentrations in the environment may have on the user's snoring probability. The hub device can then generate recommendations to adjust CO₂ concentrations in the environment to reduce or prevent the user from snoring.

Sometimes, the hub device can also learn about conditions in the environment to predict moments where sleep may be disturbed. For example, if there is a regular level of noise in the neighborhood, the hub device can determine that such noise should be masked to protect the user's sleep. The hub device can also provide suggestions to the user about how to prepare the environment, such as their bedroom, for improved sleep. For example, the hub device can generate recommendations such as opening windows for 1 hour between 3 PM and 5 PM every day. The hub device can also determine an ambient quality score (e.g., on a scale of 0-100) that can correspond to improved, restful sleep for the particular user. The hub device can further use indoor air quality data (e.g., CO₂, VOC, temperature, humidity, barometric pressure, etc.) sensed by the hub device and/or other components in the environment to determine sleep quality detriments. The hub device can determine sleep improvement and/or disturbances based on weather and/or pressure. The hub device can also track how ambient pressure correlates to local weather and the particular user's sleep patterns.

The hub device may assist the user in falling asleep by masking pink noise, thereby improving sleep quality of the user. The hub device can also improve conditions in the ambient environment for each sleep stage to improve sleep quality of the user. Sometimes, the hub device can connect screen time on mobile devices to sleep quality. The hub device can keep track of when the mobile devices are being used in bed and for how long. After all, the mobile devices may be proxies for light pollution, even if the lights are off in the environment. The hub device can determine how this light pollution affects the user's ability to fall asleep (e.g., how long it takes to fall asleep) and how well the user sleeps (e.g., sleep quality).

The hub device can provide various sleep insights to the user. The hub device can generate automatic reports that associate environmental conditions with types of sleep. The hub device can also detect detrimental ambient attributes (e.g., noise, light, etc.) and alleviate such attributes with smart integration and home automation (e.g., white noise, opening and closing windows, etc.). Although the hub device may detect noise levels in the environment, the hub device can adjust sound recordings to protect privacy (e.g., by using hardware packet (HW) filtering) while enabling detection of noises to identify sleep apnea, snoring, or disturbing levels of noise that affect sleep quality. For example, to protect user privacy, HW filtering provides for low-pass filtering the sound recordings with a cutoff frequency of approximately 2 KHz since consonants (which are essential to make intelligible understandings of what is being spoken) can be mainly present in a frequency band above 2 KHz.

The hub device can perform additional functionality. For example, the hub device can correlate movement and changes in pressure on the bed to sleep stages. Using this information, the hub device can more accurately determine sleep quality of the user. The hub device can employ algorithms that fuse together and correlate various different sensor values in order to generate more accurate information about the user, such as their sleep positioning. The hub device can also log and track instances of positional changes, rolling over, and other movement that may occur during a sleep cycle and that may impact sleep quality.

Communication with various different devices, systems, and services can also provide for improved monitoring of environmental, health, and sleep metrics. For example, the hub device can enable connection with third party services, such as HEPA filters and/or home HVAC fans in order to change conditions in the environment that affect sleep quality. The hub device may also detect ambient lighting and use sleep data to determine automated controls for shutters or other blinds in the environment. The hub device can also use CO₂, temperature, humidity, and sleep data to automate windows, heating, and/or AC in the environment. Sometimes, the hub device may provide for internet of things (IoT) linking of a nightstand to smart lights to enhance or otherwise improve the user's wakeup experience. Even more so, the hub device can enable users to copy and paste (or otherwise mirror) desired conditions in their home to a hotel room or other environment that they are located in. The hub device can provide for management of smart home features (e.g., lights, door locks, blinds, etc.) that can be based on the user's ability to fall asleep, remain asleep, and wake up. The hub device can also connect to other devices (e.g., WiFi, BLUETOOTH, and/or USB connection) in the user's environment to provide interaction and functionality through the hub device.

Moreover, functionality of the hub device can provide for visualizing information about the particular user and populations of users. For example, the hub device can provide an additional dimension for clustering similar users. The hub device can identify how users sleep in relation to other users of a same age and/or region (e.g., similar environment, similar meteorological conditions, etc.). Similar environment conditions can also be mapped across different regions to cluster users into different groupings and to generate additional insight into sleep quality of those clustered users.

The hub device can provide a variety of visualizations to the user. For example, histograms of sleeper parameters can be displayed at the user interface. The histograms can be relative to the particular user and/or generate across different groups of users. The hub device can also output and display bed pressure graphs and/or biometrics. The hub device can further display near RT, HR, and respiration information. Moreover, the hub device can display logged historic parameters associated with different variables in the environment (e.g., temperature, sound, air pressure, etc.).

### Example Airbed Hardware

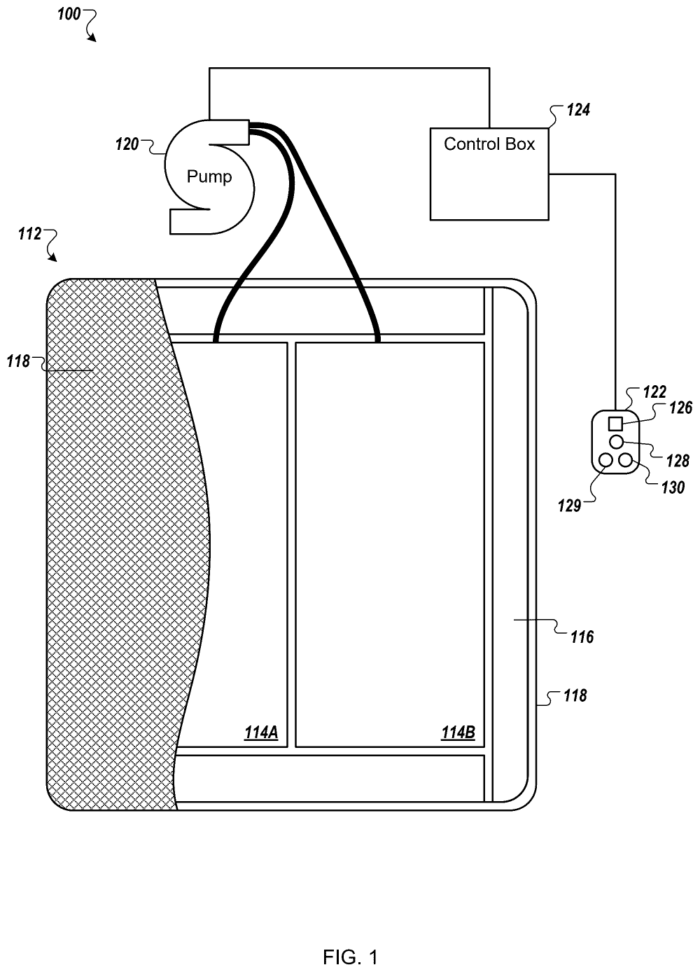

FIG. 1 shows an example air bed system 100 that includes a bed 112. The bed 112 can be a mattress that includes at least one air chamber 114 surrounded by a resilient border 116 and encapsulated by bed ticking 118. The resilient border 116 can comprise any suitable material, such as foam. In some embodiments, the resilient border 116 can combine with a top layer or layers of foam (not shown in FIG. 1) to form an upside down foam tub. In other embodiments, mattress structure can be varied as suitable for the application.

As illustrated in FIG. 1, the bed 112 can be a two chamber design having first and second fluid chambers, such as a first air chamber 114A and a second air chamber 114B. Sometimes, the bed 112 can include chambers for use with fluids other than air that are suitable for the application. For example, the fluids can include liquid. In some embodiments, such as single beds or kids' beds, the bed 112 can include a single air chamber 114A or 114B or multiple air chambers 114A and 114B. Although not depicted, sometimes, the bed 112 can include additional air chambers.

The first and second air chambers 114A and 114B can be in fluid communication with a pump 120. The pump 120 can be in electrical communication with a remote control 122 via control box 124. The control box 124 can include a wired or wireless communications interface for communicating with one or more devices, including the remote control 122. The control box 124 can be configured to operate the pump 120 to cause increases and decreases in the fluid pressure of the first and second air chambers 114A and 114B based upon commands input by a user using the remote control 122. In some implementations, the control box 124 is integrated into a housing of the pump 120. Moreover, sometimes, the pump 120 can be in wireless communication (e.g., via a home network, WIFI, BLUETOOTH, or other wireless network) with a mobile device via the control box 124. The mobile device can include but is not limited to the user's smartphone, cell phone, laptop, tablet, computer, wearable device, home automation device, or other computing device. A mobile application can be presented at the mobile device and provide functionality for the user to control the bed 112 and view information about the bed 112. The user can input commands in the mobile application presented at the mobile device. The inputted commands can be transmitted to the control box 124, which can operate the pump 120 based upon the commands.

The remote control 122 can include a display 126, an output selecting mechanism 128, a pressure increase button 129, and a pressure decrease button 130. The remote control 122 can include one or more additional output selecting mechanisms and/or buttons. The display 126 can present information to the user about settings of the bed 112. For example, the display 126 can present pressure settings of both the first and second air chambers 114A and 114B or one of the first and second air chambers 114A and 114B. Sometimes, the display 126 can be a touch screen, and can receive input from the user indicating one or more commands to control pressure in the first and second air chambers 114A and 114B and/or other settings of the bed 112.

The output selecting mechanism 128 can allow the user to switch air flow generated by the pump 120 between the first and second air chambers 114A and 114B, thus enabling control of multiple air chambers with a single remote control 122 and a single pump 120. For example, the output selecting mechanism 128 can by a physical control (e.g., switch or button) or an input control presented on the display 126. Alternatively, separate remote control units can be provided for each air chamber 114A and 114B and can each include the ability to control multiple air chambers. Pressure increase and decrease buttons 129 and 130 can allow the user to increase or decrease the pressure, respectively, in the air chamber selected with the output selecting mechanism 128. Adjusting the pressure within the selected air chamber can cause a corresponding adjustment to the firmness of the respective air chamber. In some embodiments, the remote control 122 can be omitted or modified as appropriate for an application. For example, as mentioned above, the bed 112 can be controlled by a mobile device in wired or wireless communication with the bed 112.

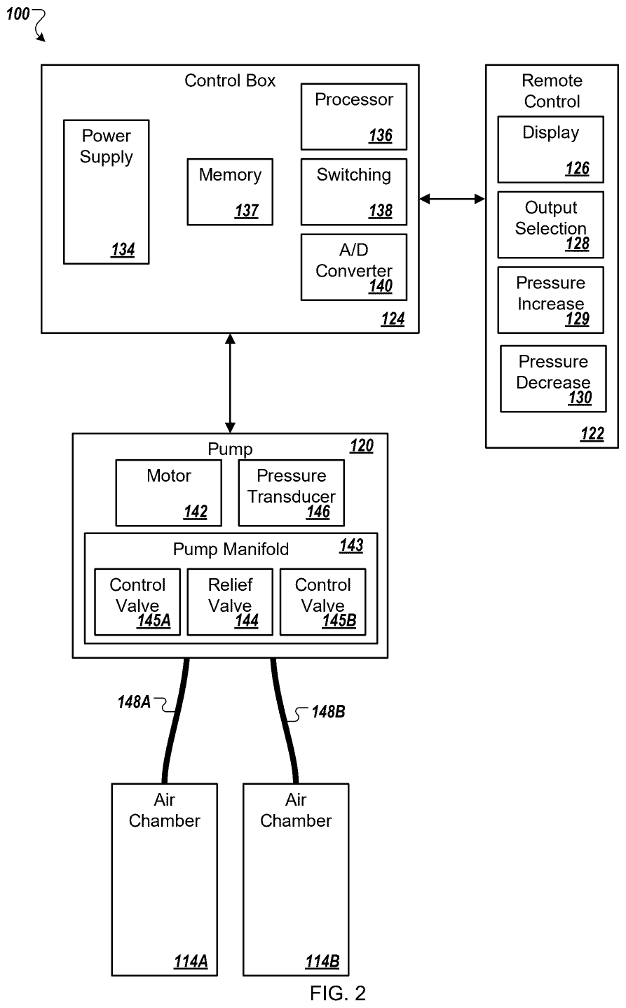

FIG. 2 is a block diagram of an example of various components of an air bed system. For example, these components can be used in the example air bed system 100. As shown in FIG. 2, the control box 124 can include a power supply 134, a processor 136, a memory 137, a switching mechanism 138, and an analog to digital (A/D) converter 140. The switching mechanism 138 can be, for example, a relay or a solid state switch. In some implementations, the switching mechanism 138 can be located in the pump 120 rather than the control box 124.

The pump 120 and the remote control 122 can be in two-way communication with the control box 124. The pump 120 includes a motor 142, a pump manifold 143, a relief valve 144, a first control valve 145A, a second control valve 145B, and a pressure transducer 146. The pump 120 is fluidly connected with the first air chamber 114A and the second air chamber 114B via a first tube 148A and a second tube 148B, respectively. The first and second control valves 145A and 145B can be controlled by switching mechanism 138, and are operable to regulate the flow of fluid between the pump 120 and first and second air chambers 114A and 114B, respectively.

In some implementations, the pump 120 and the control box 124 can be provided and packaged as a single unit. In some implementations, the pump 120 and the control box 124 can be provided as physically separate units. In yet some implementations, the control box 124, the pump 120, or both can be integrated within or otherwise contained within a bed frame, foundation, or bed support structure that supports the bed 112. Sometimes, the control box 124, the pump 120, or both can be located outside of a bed frame, foundation, or bed support structure (as shown in the example in FIG. 1).

The example air bed system 100 depicted in FIG. 2 includes the two air chambers 114A and 114B and the single pump 120 of the bed 112 depicted in FIG. 1. However, other implementations can include an air bed system having two or more air chambers and one or more pumps incorporated into the air bed system to control the air chambers. For example, a separate pump can be associated with each air chamber of the air bed system. As another example, a pump can be associated with multiple chambers of the air bed system. A first pump can, for example, be associated with air chambers that extend longitudinally from a left side to a midpoint of the air bed system 100 and a second pump can be associated with air chambers that extend longitudinally from a right side to the midpoint of the air bed system 100. Separate pumps can allow each air chamber to be inflated or deflated independently and/or simultaneously. Furthermore, additional pressure transducers can be incorporated into the air bed system 100 such that, for example, a separate pressure transducer can be associated with each air chamber.

As an illustrative example, in use, the processor 136 can send a decrease pressure command to one of air chambers 114A or 114B, and the switching mechanism 138 can convert the low voltage command signals sent by the processor 136 to higher operating voltages sufficient to operate the relief valve 144 of the pump 120 and open the respective control valve 145A or 145B. Opening the relief valve 144 can allow air to escape from the air chamber 114A or 114B through the respective air tube 148A or 148B. During deflation, the pressure transducer 146 can send pressure readings to the processor 136 via the A/D converter 140. The A/D converter 140 can receive analog information from pressure transducer 146 and can convert the analog information to digital information useable by the processor 136. The processor 136 can send the digital signal to the remote control 122 to update the display 126 in order to convey the pressure information to the user. The processor 136 can also send the digital signal to one or more other devices in wired or wireless communication with the air bed system, including but not limited to mobile devices such as smartphones, cellphones, tablets, computers, wearable devices, and home automation devices. As a result, the user can view pressure information associated with the air bed system at their mobile device instead of at, or in addition to, the remote control 122.

As another example, the processor 136 can send an increase pressure command. The pump motor 142 can be energized in response to the increase pressure command and send air to the designated one of the air chambers 114A or 114B through the air tube 148A or 148B via electronically operating the corresponding valve 145A or 145B. While air is being delivered to the designated air chamber 114A or 114B in order to increase the firmness of the chamber, the pressure transducer 146 can sense pressure within the pump manifold 143. Again, the pressure transducer 146 can send pressure readings to the processor 136 via the A/D converter 140. The processor 136 can use the information received from the A/D converter 140 to determine the difference between the actual pressure in air chamber 114A or 114B and the desired pressure. The processor 136 can send the digital signal to the remote control 122 to update display 126 in order to convey the pressure information to the user.

Generally speaking, during an inflation or deflation process, the pressure sensed within the pump manifold 143 can provide an approximation of the pressure within the respective air chamber that is in fluid communication with the pump manifold 143. An example method of obtaining a pump manifold pressure reading that is substantially equivalent to the actual pressure within an air chamber includes turning off the pump 120, allowing the pressure within the air chamber 114A or 114B and the pump manifold 143 to equalize, and then sensing the pressure within the pump manifold 143 with the pressure transducer 146. Thus, providing a sufficient amount of time to allow the pressures within the pump manifold 143 and chamber 114A or 114B to equalize can result in pressure readings that are accurate approximations of actual pressure within air chamber 114A or 114B. In some implementations, the pressure of the air chambers 114A and/or 114B can be continuously monitored using multiple pressure sensors (not shown). The pressure sensors can be positioned within the air chambers 114A and/or 114B. The pressure sensors can also be fluidly connected to the air chambers 114A and 114B, such as along the air tubes 148A and 148B.

In some implementations, information collected by the pressure transducer 146 can be analyzed to determine various states of a user laying on the bed 112. For example, the processor 136 can use information collected by the pressure transducer 146 to determine a heartrate or a respiration rate for the user laying on the bed 112. As an illustrative example, the user can be laying on a side of the bed 112 that includes the chamber 114A. The pressure transducer 146 can monitor fluctuations in pressure of the chamber 114A, and this information can be used to determine the user's heartrate and/or respiration rate. As another example, additional processing can be performed using the collected data to determine a sleep state of the user (e.g., awake, light sleep, deep sleep). For example, the processor 136 can determine when the user falls asleep and, while asleep, the various sleep states (e.g., sleep stages) of the user. Based on the determined heartrate, respiration rate, and/or sleep states of the user, the processor 136 can determine information about the user's sleep quality. The processor 136 can, for example, determine how well the user slept during a particular sleep cycle. The processor 136 can also determine user sleep cycle trends. Accordingly, the processor 136 can generate recommendations to improve the user's sleep quality and overall sleep cycle. Information that is determined about the user's sleep cycle (e.g., heartrate, respiration rate, sleep states, sleep quality, recommendations to improve sleep quality, etc.) can be transmitted to the user's mobile device and presented in a mobile application, as described above.

Additional information associated with the user of the air bed system 100 that can be determined using information collected by the pressure transducer 146 includes motion of the user, presence of the user on a surface of the bed 112, weight of the user, heart arrhythmia of the user, snoring of the user or another user on the air bed system, and apnea of the user. One or more other health conditions of the user can also be determined based on the information collected by the pressure transducer 146. Taking user presence detection for example, the pressure transducer 146 can be used to detect the user's presence on the bed 112, e.g., via a gross pressure change determination and/or via one or more of a respiration rate signal, heartrate signal, and/or other biometric signals. Detection of the user's presence on the bed 112 can be beneficial to determine, by the processor 136, one or more adjustments to make to settings of the bed 112 (e.g., adjusting a firmness of the bed 112 when the user is present to a user-preferred firmness setting) and/or peripheral devices (e.g., turning off lights when the user is present, activating a heating or cooling system, etc.).

For example, a simple pressure detection process can identify an increase in pressure as an indication that the user is present on the bed 112. As another example, the processor 136 can determine that the user is present on the bed 112 if the detected pressure increases above a specified threshold (so as to indicate that a person or other object above a certain weight is positioned on the bed 112). As yet another example, the processor 136 can identify an increase in pressure in combination with detected slight, rhythmic fluctuations in pressure as corresponding to the user being present on the bed 112. The presence of rhythmic fluctuations can be identified as being caused by respiration or heart rhythm (or both) of the user. The detection of respiration or a heartbeat can distinguish between the user being present on the bed and another object (e.g., a suitcase, a pet, a pillow, etc.) being placed upon the bed.

In some implementations, fluctuations in pressure can be measured at the pump 120. For example, one or more pressure sensors can be located within one or more internal cavities of the pump 120 to detect fluctuations in pressure within the pump 120. The fluctuations in pressure detected at the pump 120 can indicate fluctuations in pressure in one or both of the chambers 114A and 114B. One or more sensors located at the pump 120 can be in fluid communication with one or both of the chambers 114A and 114B, and the sensors can be operative to determine pressure within the chambers 114A and 114B. The control box 124 can be configured to determine at least one vital sign (e.g., heartrate, respiratory rate) based on the pressure within the chamber 114A or the chamber 114B.

In some implementations, the control box 124 can analyze a pressure signal detected by one or more pressure sensors to determine a heartrate, respiration rate, and/or other vital signs of the user lying or sitting on the chamber 114A and/or 114B. More specifically, when a user lies on the bed 112 and is positioned over the chamber 114A, each of the user's heart beats, breaths, and other movements (e.g., hand, arm, leg, foot, or other gross body movements) can create a force on the bed 112 that is transmitted to the chamber 114A. As a result of the force input applied to the chamber 114A from the user's movement, a wave can propagate through the chamber 114A and into the pump 120. A pressure sensor located at the pump 120 can detect the wave, and thus the pressure signal outputted by the sensor can indicate a heartrate, respiratory rate, or other information regarding the user.

With regard to sleep state, the air bed system 100 can determine the user's sleep state by using various biometric signals such as heartrate, respiration, and/or movement of the user. While the user is sleeping, the processor 136 can receive one or more of the user's biometric signals (e.g., heartrate, respiration, motion, etc.) and can determine the user's present sleep state based on the received biometric signals. In some implementations, signals indicating fluctuations in pressure in one or both of the chambers 114A and 114B can be amplified and/or filtered to allow for more precise detection of heartrate and respiratory rate.

Sometimes, the processor 136 can also receive additional biometric signals of the user from one or more other sensors or sensor arrays that are positioned on or otherwise integrated into the air bed system 100. For example, one or more sensors can be attached or removably attached to a top surface of the air bed system 100 and configured to detect signals such as heartrate, respiration rate, and/or motion of the user. The processor 136 can then combine biometric signals received from pressure sensors located at the pump 120, the pressure transducer 146, and/or the sensors positioned throughout the air bed system 100 to generate accurate and more precise heartrate, respiratory rate, and other information about the user and the user's sleep quality.

Sometimes, the control box 124 can perform a pattern recognition algorithm or other calculation based on the amplified and filtered pressure signal(s) to determine the user's heartrate and/or respiratory rate. For example, the algorithm or calculation can be based on assumptions that a heartrate portion of the signal has a frequency in a range of 0.5-4.0 Hz and that a respiration rate portion of the signal has a frequency in a range of less than 1 Hz. Sometimes, the control box 124 can use one or more machine learning models to determine the user's heartrate, respiratory rate, or other health information. The models can be trained using training data that includes training pressure signals and expected heartrates and/or respiratory rates. Sometimes, the control box 124 can determine the user's heartrate, respiratory rate, or other health information by using a lookup table that corresponds to sensed pressure signals.

The control box 124 can also be configured to determine other characteristics of the user based on the received pressure signal, such as blood pressure, tossing and turning movements, rolling movements, limb movements, weight, presence or lack of presence of the user, and/or the identity of the user.

For example, the pressure transducer 146 can be used to monitor the air pressure in the chambers 114A and 114B of the bed 112. If the user on the bed 112 is not moving, the air pressure changes in the air chamber 114A or 114B can be relatively minimal, and can be attributable to respiration and/or heartbeat. When the user on the bed 112 is moving, however, the air pressure in the mattress can fluctuate by a much larger amount. Thus, the pressure signals generated by the pressure transducer 146 and received by the processor 136 can be filtered and indicated as corresponding to motion, heartbeat, or respiration. The processor 136 can also attribute such fluctuations in air pressure to sleep quality of the user. Such attributions can be determined based on applying one or more machine learning models and/or algorithms to the pressure signals generated by the pressure transducer 146. For example, if the user shifts and turns a lot during a sleep cycle (for example, in comparison to historic trends of the user's sleep cycles), the processor 136 can determine that the user experienced poor sleep during that particular sleep cycle.

In some implementations, rather than performing the data analysis in the control box 124 with the processor 136, a digital signal processor (DSP) can be provided to analyze the data collected by the pressure transducer 146. Alternatively, the data collected by the pressure transducer 146 can be sent to a cloud-based computing system for remote analysis.

In some implementations, the example air bed system 100 further includes a temperature controller configured to increase, decrease, or maintain a temperature of the bed 112, for example for the comfort of the user. For example, a pad (e.g., mat, layer, etc.) can be placed on top of or be part of the bed 112, or can be placed on top of or be part of one or both of the chambers 114A and 114B. Air can be pushed through the pad and vented to cool off the user on the bed 112. Additionally or alternatively, the pad can include a heating element that can be used to keep the user warm. In some implementations, the temperature controller can receive temperature readings from the pad. The temperature controller can determine whether the temperature readings are less than or greater than some threshold range and/or value. Based on this determination, the temperature controller can actuate components to push air through the pad to cool off the user or active the heating element. In some implementations, separate pads are used for different sides of the bed 112 (e.g., corresponding to the locations of the chambers 114A and 114B) to provide for differing temperature control for the different sides of the bed 112. Each pad can therefore be selectively controlled by the temperature controller to provide cooling or heating that is preferred by each of the users on the different sides of the bed 112. For example, a first user on a left side of the bed 112 can prefer to have their side of the bed 112 cooled during the night while a second user on a right side of the bed 112 can prefer to have their side of the bed 112 warmed during the night.

In some implementations, the user of the air bed system 100 can use an input device, such as the remote control 122 or a mobile device as described above, to input a desired temperature for a surface of the bed 112 (or for a portion of the surface of the bed 112, for example at a foot region, a lumbar or waist region, a shoulder region, and/or a head region of the bed 112). The desired temperature can be encapsulated in a command data structure that includes the desired temperature and also identifies the temperature controller as the desired component to be controlled. The command data structure can then be transmitted via Bluetooth or another suitable communication protocol (e.g., WIFI, a local network, etc.) to the processor 136. In various examples, the command data structure is encrypted before being transmitted. The temperature controller can then configure its elements to increase or decrease the temperature of the pad depending on the temperature input provided at the remote control 122 by the user.

In some implementations, data can be transmitted from a component back to the processor 136 or to one or more display devices, such as the display 126 of the remote controller 122. For example, the current temperature as determined by a sensor element of temperature controller, the pressure of the bed, the current position of the foundation or other information can be transmitted to control box 124. The control box 124 can then transmit the received information to the remote control 122, where the information can be displayed to the user (e.g., on the display 126). As described above, the control box 124 can also transmit the received information to a mobile device (e.g., smartphone, cellphone, laptop, tablet, computer, wearable device, or home automation device) to be displayed in a mobile application or other graphical user interface (GUI) to the user.

In some implementations, the example air bed system 100 further includes an adjustable foundation and an articulation controller configured to adjust the position of a bed (e.g., the bed 112) by adjusting the adjustable foundation that supports the bed. For example, the articulation controller can adjust the bed 112 from a flat position to a position in which a head portion of a mattress of the bed is inclined upward (e.g., to facilitate a user sitting up in bed and/or watching television). The bed 112 can also include multiple separately articulable sections. As an illustrative example, the bed 112 can include one or more of a head portion, a lumbar/waist portion, a leg portion, and/or a foot portion, all of which can be separately articulable. As another example, portions of the bed 112 corresponding to the locations of the chambers 114A and 114B can be articulated independently from each other, to allow one user positioned on the bed 112 surface to rest in a first position (e.g., a flat position or other desired position) while a second user rests in a second position (e.g., a reclining position with the head raised at an angle from the waist or another desired position). Separate positions can also be set for two different beds (e.g., two twin beds placed next to each other). The foundation of the bed 112 can include more than one zone that can be independently adjusted.

Sometimes, the bed 112 can be adjusted to one or more user-defined positions based on user input and/or user preferences. For example, the bed 112 can automatically adjust, by the articulation controller, to one or more user-defined settings. As another example, the user can control the articulation controller to adjust the bed 112 to one or more user-defined positions. Sometimes, the bed 112 can be adjusted to one or more positions that may provide the user with improved or otherwise improve sleep and sleep quality. For example, a head portion on one side of the bed 112 can be automatically articulated, by the articulation controller, when one or more sensors of the air bed system 100 detect that a user sleeping on that side of the bed 112 is snoring. As a result, the user's snoring can be mitigated so that the snoring does not wake up another user sleeping in the bed 112.

In some implementations, the bed 112 can be adjusted using one or more devices in communication with the articulation controller or instead of the articulation controller. For example, the user can change positions of one or more portions of the bed 112 using the remote control 122 described above. The user can also adjust the bed 112 using a mobile application or other graphical user interface presented at a mobile computing device of the user.

The articulation controller can also be configured to provide different levels of massage to one or more portions of the bed 112 for one or more users on the bed 112. The user(s) can also adjust one or more massage settings for different portions of the bed 112 using the remote control 122 and/or a mobile device in communication with the air bed system 100, as described above.

### Example of a Bed in a Bedroom Environment

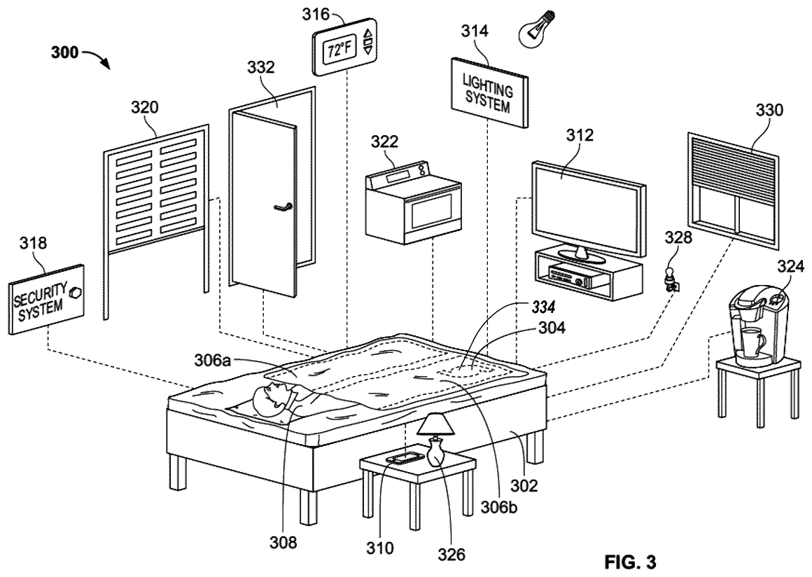

FIG. 3 shows an example environment 300 including a bed 302 in communication with devices located in and around a home. In the example shown, the bed 302 includes pump 304 for controlling air pressure within two air chambers 306a and 306b (as described above with respect to the air chambers 114A and 114B). The pump 304 additionally includes circuitry 334 for controlling inflation and deflation functionality performed by the pump 304. The circuitry 334 is further programmed to detect fluctuations in air pressure of the air chambers 306a-b and uses the detected fluctuations in air pressure to identify bed presence of a user 308, sleep state of the user 308, movement of the user 308, and biometric signals of the user 308, such as heartrate and respiration rate. The detected fluctuations in air pressure can also be used to detect when the user 308 is snoring and whether the user 308 has sleep apnea or other health conditions. Moreover, the detected fluctuations in air pressure can be used to determine an overall sleep quality of the user 308.

In the example shown, the pump 304 is located within a support structure of the bed 302 and the control circuitry 334 for controlling the pump 304 is integrated with the pump 304. In some implementations, the control circuitry 334 is physically separate from the pump 304 and is in wireless or wired communication with the pump 304. In some implementations, the pump 304 and/or control circuitry 334 are located outside of the bed 302. In some implementations, various control functions can be performed by systems located in different physical locations. For example, circuitry for controlling actions of the pump 304 can be located within a pump casing of the pump 304 while control circuitry 334 for performing other functions associated with the bed 302 can be located in another portion of the bed 302, or external to the bed 302. As another example, the control circuitry 334 located within the pump 304 can communicate with control circuitry 334 at a remote location through a LAN or WAN (e.g., the internet). As yet another example, the control circuitry 334 can be included in the control box 124 of FIGS. 1 and 2.

In some implementations, one or more devices other than, or in addition to, the pump 304 and control circuitry 334 can be utilized to identify user bed presence, sleep state, movement, biometric signals, and other information (e.g., sleep quality and/or health related) about the user 308. For example, the bed 302 can include a second pump in addition to the pump 304, with each of the two pumps connected to a respective one of the air chambers 306a-b. For example, the pump 304 can be in fluid communication with the air chamber 306b to control inflation and deflation of the air chamber 306b as well as detect user signals for a user located over the air chamber 306b, such as bed presence, sleep state, movement, and biometric signals. The second pump can then be in fluid communication with the air chamber 306a and used to control inflation and deflation of the air chamber 306a as well as detect user signals for a user located over the air chamber 306a.

As another example, the bed 302 can include one or more pressure sensitive pads or surface portions that are operable to detect movement, including user presence, user motion, respiration, and heartrate. A first pressure sensitive pad can be incorporated into a surface of the bed 302 over a left portion of the bed 302, where a first user would normally be located during sleep, and a second pressure sensitive pad can be incorporated into the surface of the bed 302 over a right portion of the bed 302, where a second user would normally be located during sleep. The movement detected by the one or more pressure sensitive pads or surface portions can be used by control circuitry 334 to identify user sleep state, bed presence, or biometric signals for each of the users. The pressure sensitive pads can also be removable rather than incorporated into the surface of the bed 302.

The bed 302 can also include one or more temperature sensors and/or array of sensors that are operable to detect temperatures in microclimates of the bed 302. Detected temperatures in different microclimates of the bed 302 can be used by the control circuitry 334 to determine one or more modifications to the user 308's sleep environment. For example, a temperature sensor located near a core region of the bed 302 where the user 308 rests can detect high temperature values. Such high temperature values can indicate that the user 308 is warm. To lower the user's body temperature in this microclimate, the control circuitry 334 can determine that a cooling element of the bed 302 can be activated. As another example, the control circuitry 334 can determine that a cooling unit in the home can be automatically activated to cool an ambient temperature in the environment 300.

The control circuitry 334 can also process a combination of signals sensed by different sensors that are integrated into, positioned on, or otherwise in communication with the bed 112. For example, pressure and temperature signals can be processed by the control circuitry 334 to more accurately determine one or more health conditions of the user 308 and/or sleep quality of the user 308. Acoustic signals detected by one or more microphones or other audio sensors can also be used in combination with pressure or motion sensors in order to determine when the user 308 snores, whether the user 308 has sleep apnea, and/or overall sleep quality of the user 308. Combinations of one or more other sensed signals are also possible for the control circuitry 334 to more accurately determine one or more health and/or sleep conditions of the user 308.

Accordingly, information detected by one or more sensors or other components of the bed 112 (e.g., motion information) can be processed by the control circuitry 334 and provided to one or more user devices, such as a user device 310 for presentation to the user 308 or to other users. The information can be presented in a mobile application or other graphical user interface at the user device 310. The user 308 can view different information that is processed and/or determined by the control circuitry 334 and based the signals that are detected by components of the bed 302. For example, the user 308 can view their overall sleep quality for a particular sleep cycle (e.g., the previous night), historic trends of their sleep quality, and health information. The user 308 can also adjust one or more settings of the bed 302 (e.g., increase or decrease pressure in one or more regions of the bed 302, incline or decline different regions of the bed 302, turn on or off massage features of the bed 302, etc.) using the mobile application that is presented at the user device 310.

In the example depicted in FIG. 3, the user device 310 is a mobile phone; however, the user device 310 can also be any one of a tablet, personal computer, laptop, a smartphone, a smart television (e.g., a television 312), a home automation device, or other user device capable of wired or wireless communication with the control circuitry 334, one or more other components of the bed 302, and/or one or more devices in the environment 300. The user device 310 can be in communication with the control circuitry 334 of the bed 302 through a network or through direct point-to-point communication. For example, the control circuitry 334 can be connected to a LAN (e.g., through a WIFI router) and communicate with the user device 310 through the LAN. As another example, the control circuitry 334 and the user device 310 can both connect to the Internet and communicate through the Internet. For example, the control circuitry 334 can connect to the Internet through a WIFI router and the user device 310 can connect to the Internet through communication with a cellular communication system. As another example, the control circuitry 334 can communicate directly with the user device 310 through a wireless communication protocol, such as Bluetooth. As yet another example, the control circuitry 334 can communicate with the user device 310 through a wireless communication protocol, such as ZigBee, Z-Wave, infrared, or another wireless communication protocol suitable for the application. As another example, the control circuitry 334 can communicate with the user device 310 through a wired connection such as, for example, a USB connector, serial/RS232, or another wired connection suitable for the application.

As mentioned above, the user device 310 can display a variety of information and statistics related to sleep, or user 308's interaction with the bed 302. For example, a user interface displayed by the user device 310 can present information including amount of sleep for the user 308 over a period of time (e.g., a single evening, a week, a month, etc.), amount of deep sleep, ratio of deep sleep to restless sleep, time lapse between the user 308 getting into bed and the user 308 falling asleep, total amount of time spent in the bed 302 for a given period of time, heartrate for the user 308 over a period of time, respiration rate for the user 308 over a period of time, or other information related to user interaction with the bed 302 by the user 308 or one or more other users of the bed 302. In some implementations, information for multiple users can be presented on the user device 310, for example information for a first user positioned over the air chamber 306a can be presented along with information for a second user positioned over the air chamber 306b. In some implementations, the information presented on the user device 310 can vary according to the age of the user 308. For example, the information presented on the user device 310 can evolve with the age of the user 308 such that different information is presented on the user device 310 as the user 308 ages as a child or an adult.

The user device 310 can also be used as an interface for the control circuitry 334 of the bed 302 to allow the user 308 to enter information and/or adjust one or more settings of the bed 302. The information entered by the user 308 can be used by the control circuitry 334 to provide better information to the user 308 or to various control signals for controlling functions of the bed 302 or other devices. For example, the user 308 can enter information such as weight, height, and age of the user 308. The control circuitry 334 can use this information to provide the user 308 with a comparison of the user 308's tracked sleep information to sleep information of other people having similar weights, heights, and/or ages as the user 308. The control circuitry 308 can also use this information to more accurately determine overall sleep quality and/or health of the user 308 based on information that is detected by one or more components (e.g., sensors) of the bed 302.

As another example, and as mentioned above, the user 308 can use the user device 310 as an interface for controlling air pressure of the air chambers 306a and 306b, for controlling various recline or incline positions of the bed 302, for controlling temperature of one or more surface temperature control devices of the bed 302, or for allowing the control circuitry 334 to generate control signals for other devices (as described in greater detail below).

In some implementations, the control circuitry 334 of the bed 302 can communicate with other devices or systems in addition to or instead of the user device 310. For example, the control circuitry 334 can communicate with the television 312, a lighting system 314, a thermostat 316, a security system 318, home automation devices, and/or other household devices, including but not limited to an oven 322, a coffee maker 324, a lamp 326, and/or a nightlight 328. Other examples of devices and/or systems that the control circuitry 334 can communicate with include a system for controlling window blinds 330, one or more devices for detecting or controlling the states of one or more doors 332 (such as detecting if a door is open, detecting if a door is locked, or automatically locking a door), and a system for controlling a garage door 320 (e.g., control circuitry 334 integrated with a garage door opener for identifying an open or closed state of the garage door 320 and for causing the garage door opener to open or close the garage door 320). Communications between the control circuitry 334 of the bed 302 and other devices can occur through a network (e.g., a LAN or the Internet) or as point-to-point communication (e.g., using Bluetooth, radio communication, or a wired connection). In some implementations, control circuitry 334 of different beds 302 can communicate with different sets of devices. For example, a kid's bed may not communicate with and/or control the same devices as an adult bed. In some embodiments, the bed 302 can evolve with the age of the user such that the control circuitry 334 of the bed 302 communicates with different devices as a function of age of the user of that bed 302.

The control circuitry 334 can receive information and inputs from other devices/systems and use the received information and inputs to control actions of the bed 302 and/or other devices. For example, the control circuitry 334 can receive information from the thermostat 316 indicating a current environmental temperature for a house or room in which the bed 302 is located. The control circuitry 334 can use the received information (along with other information, such as signals detected from one or more sensors of the bed 302) to determine if a temperature of all or a portion of the surface of the bed 302 should be raised or lowered. The control circuitry 334 can then cause a heating or cooling mechanism of the bed 302 to raise or lower the temperature of the surface of the bed 302. The control circuitry 334 can also cause a heating or cooling unit of the house or room in which the bed 302 is located to raise or lower the ambient temperature surrounding the bed 302. Thus, by adjusting the temperature of the bed 302 and/or the room in which the bed 302 is located, the user 308 can experience more improved sleep quality and comfort.

As an example, the user 308 can indicate a desired sleeping temperature of 74 degrees while a second user of the bed 302 indicates a desired sleeping temperature of 72 degrees. The thermostat 316 can transmit signals indicating room temperature at predetermined times to the control circuitry 334. The thermostat 316 can also send a continuous stream of detected temperature values of the room to the control circuitry 334. The transmitted signal(s) can indicate to the control circuitry 334 that the current temperature of the bedroom is 72 degrees. The control circuitry 334 can identify that the user 308 has indicated a desired sleeping temperature of 74 degrees, and can accordingly send control signals to a heating pad located on the user 308's side of the bed to raise the temperature of the portion of the surface of the bed 302 where the user 308 is located until the user 308's desired temperature is achieved. Moreover, the control circuitry 334 can sent control signals to the thermostat 316 and/or a heating unit in the house to raise the temperature in the room in which the bed 302 is located.

The control circuitry 334 can generate control signals to control other devices and propagate the control signals to the other devices. In some implementations, the control signals are generated based on information collected by the control circuitry 334, including information related to user interaction with the bed 302 by the user 308 and/or one or more other users. Information collected from one or more other devices other than the bed 302 can also be used when generating the control signals. For example, information relating to environmental occurrences (e.g., environmental temperature, environmental noise level, and environmental light level), time of day, time of year, day of the week, or other information can be used when generating control signals for various devices in communication with the control circuitry 334 of the bed 302.

For example, information on the time of day can be combined with information relating to movement and bed presence of the user 308 to generate control signals for the lighting system 314. The control circuitry 334 can, based on detected pressure signals of the user 308 on the bed 302, determine when the user 308 is presently in the bed 302 and when the user 308 falls asleep. Once the control circuitry 334 determines that the user has fallen asleep, the control circuitry 334 can transmit control signals to the lighting system 314 to turn off lights in the room in which the bed 302 is located, to lower the window blinds 330 in the room, and/or to activate the nightlight 328. Moreover, the control circuitry 334 can receive input from the user 308 (e.g., via the user device 310) that indicates a time at which the user 308 would like to wake up. When that time approaches, the control circuitry 334 can transmit control signals to one or more devices in the environment 300 to control devices that may cause the user 308 to wake up. For example, the control signals can be sent to a home automation device that controls multiple devices in the home. The home automation device can be instructed, by the control circuitry 334, to raise the window blinds 330, turn off the nightlight 328, turn on lighting beneath the bed 302, start the coffee machine 324, change a temperature in the house via the thermostat 316, or perform some other home automation. The home automation device can also be instructed to activate an alarm that can cause the user 308 to wake up. Sometimes, the user 308 can input information at the user device 310 that indicates what actions can be taken by the home automation device or other devices in the environment 300.

In some implementations, rather than or in addition to providing control signals for one or more other devices, the control circuitry 334 can provide collected information (e.g., information related to user movement, bed presence, sleep state, or biometric signals for the user 308) to one or more other devices to allow the one or more other devices to utilize the collected information when generating control signals. For example, the control circuitry 334 of the bed 302 can provide information relating to user interactions with the bed 302 by the user 308 to a central controller (not shown) that can use the provided information to generate control signals for various devices, including the bed 302.

The central controller can, for example, be a hub device that provides a variety of information about the user 308 and control information associated with the bed 302 and one or more other devices in the house. The central controller can include one or more sensors that detect signals that can be used by the control circuitry 334 and/or the central controller to determine information about the user 308 (e.g., biometric or other health data, sleep quality, etc.). The sensors can detect signals including but not limited to ambient light, temperature, humidity, volatile organic compound(s), pulse, motion, and audio. These signals can be combined with signals that are detected by sensors of the bed 302 to determine more accurate information about the user 308's health and sleep quality. The central controller can provide controls (e.g., user-defined, presets, automated, user initiated, etc.) for the bed 302, determining and viewing sleep quality and health information, a smart alarm clock, a speaker or other home automation device, a smart picture frame, a nightlight, and one or more mobile applications that the user 308 can install and use at the central controller. The central controller can include a display screen that can output information and also receive input from the user 308. The display can output information such as the user 308's health, sleep quality, weather information, security integration features, lighting integration features, heating and cooling integration features, and other controls to automate devices in the house. The central controller can therefore operate to provide the user 308 with functionality and control of multiple different types of devices in the house as well as the user 308's bed 302.

Still referring to FIG. 3, the control circuitry 334 of the bed 302 can generate control signals for controlling actions of other devices, and transmit the control signals to the other devices in response to information collected by the control circuitry 334, including bed presence of the user 308, sleep state of the user 308, and other factors. For example, the control circuitry 334 integrated with the pump 304 can detect a feature of a mattress of the bed 302, such as an increase in pressure in the air chamber 306b, and use this detected increase in air pressure to determine that the user 308 is present on the bed 302. In some implementations, the control circuitry 334 can identify a heartrate or respiratory rate for the user 308 to identify that the increase in pressure is due to a person sitting, laying, or otherwise resting on the bed 302, rather than an inanimate object (such as a suitcase) having been placed on the bed 302. In some implementations, the information indicating user bed presence can be combined with other information to identify a current or future likely state for the user 308. For example, a detected user bed presence at 11:00 am can indicate that the user is sitting on the bed (e.g., to tie her shoes, or to read a book) and does not intend to go to sleep, while a detected user bed presence at 10:00 pm can indicate that the user 308 is in bed for the evening and is intending to fall asleep soon. As another example, if the control circuitry 334 detects that the user 308 has left the bed 302 at 6:30 am (e.g., indicating that the user 308 has woken up for the day), and then later detects presence of the user 308 at 7:30 am on the bed 302, the control circuitry 334 can use this information that the newly detected presence is likely temporary (e.g., while the user 308 ties her shoes before heading to work) rather than an indication that the user 308 is intending to stay on the bed 302 for an extended period of time.

If the control circuitry 334 determines that the user 308 is likely to remain on the bed 302 for an extended period of time, the control circuitry 334 can determine one or more home automation controls that can aid the user 308 in falling asleep and experiencing improved sleep quality throughout the user 308's sleep cycle. For example, the control circuitry 334 can communicate with security system 318 to ensure that doors are locked. The control circuitry 334 can communicate with the oven 322 to ensure that the oven 322 is turned off. The control circuitry 334 can also communicate with the lighting system 314 to dim or otherwise turn off lights in the room in which the bed 302 is located and/or throughout the house, and the control circuitry 334 can communicate with the thermostat 316 to ensure that the house is at a desired temperature of the user 308. The control circuitry 334 can also determine one or more adjustments that can be made to the bed 302 to facilitate the user 308 falling asleep and staying asleep (e.g., changing a position of one or more regions of the bed 302, foot warming, massage features, pressure/firmness in one or more regions of the bed 302, etc.).

In some implementations, the control circuitry 334 is able to use collected information (including information related to user interaction with the bed 302 by the user 308, as well as environmental information, time information, and input received from the user 308) to identify use patterns for the user 308. For example, the control circuitry 334 can use information indicating bed presence and sleep states for the user 308 collected over a period of time to identify a sleep pattern for the user. The control circuitry 334 can identify that the user 308 generally goes to bed between 9:30 pm and 10:00 pm, generally falls asleep between 10:00 pm and 11:00 pm, and generally wakes up between 6:30 am and 6:45 am, based on information indicating user presence and biometrics for the user 308 collected over a week or a different time period. The control circuitry 334 can use identified patterns of the user 308 to better process and identify user interactions with the bed 302.

For example, given the above example user bed presence, sleep, and wake patterns for the user 308, if the user 308 is detected as being on the bed 302 at 3:00 pm, the control circuitry 334 can determine that the user 308's presence on the bed 302 is only temporary, and use this determination to generate different control signals than would be generated if the control circuitry 334 determined that the user 308 was in bed for the evening (e.g., at 3:00 pm, a head region of the bed 302 can be raised to facilitate reading or watching TV while in the bed 302, whereas in the evening, the bed 302 can be adjusted to a flat position to facilitate falling asleep). As another example, if the control circuitry 334 detects that the user 308 has gotten out of bed at 3:00 am, the control circuitry 334 can use identified patterns for the user 308 to determine that the user has only gotten up temporarily (e.g., to use the bathroom, or get a glass of water) and is not up for the day. For example, the control circuitry 334 can turn on underbed lighting to assist the user 308 in carefully moving around the bed 302 and the room. By contrast, if the control circuitry 334 identifies that the user 308 has gotten out of the bed 302 at 6:40 am, the control circuitry 334 can determine that the user 308 is up for the day and generate a different set of control signals than those that would be generated if it were determined that the user 308 were only getting out of bed temporarily (as would be the case when the user 308 gets out of the bed 302 at 3:00 am) (e.g., the control circuitry 334 can turn on light 326 near the bed 302 and/or raise the window blinds 330 when it is determined that the user 308 is up for the day). For other users, getting out of the bed 302 at 3:00 am can be a normal wake-up time, which the control circuitry 334 can learn and respond to accordingly. Moreover, if the bed 302 is occupied by two users, the control circuitry 334 can learn and respond to the patterns of each of the users.

As described above, the control circuitry 334 for the bed 302 can generate control signals for control functions of various other devices. The control signals can be generated, at least in part, based on detected interactions by the user 308 with the bed 302, as well as other information including time, date, temperature, etc. The control circuitry 334 can communicate with the television 312, receive information from the television 312, and generate control signals for controlling functions of the television 312. For example, the control circuitry 334 can receive an indication from the television 312 that the television 312 is currently turned on. If the television 312 is located in a different room than the bed 302, the control circuitry 334 can generate a control signal to turn the television 312 off upon making a determination that the user 308 has gone to bed for the evening or otherwise is remaining in the room with the bed 302. For example, if presence of the user 308 is detected on the bed 302 during a particular time range (e.g., between 8:00 pm and 7:00 am) and persists for longer than a threshold period of time (e.g., 10 minutes), the control circuitry 334 can determine that the user 308 is in bed for the evening. If the television 312 is on (as indicated by communications received by the control circuitry 334 of the bed 302 from the television 312), the control circuitry 334 can generate a control signal to turn the television 312 off. The control signals can be transmitted to the television (e.g., through a directed communication link between the television 312 and the control circuitry 334 or through a network, such as WIFI). As another example, rather than turning off the television 312 in response to detection of user bed presence, the control circuitry 334 can generate a control signal that causes the volume of the television 312 to be lowered by a pre-specified amount.

As another example, upon detecting that the user 308 has left the bed 302 during a specified time range (e.g., between 6:00 am and 8:00 am), the control circuitry 334 can generate control signals to cause the television 312 to turn on and tune to a pre-specified channel (e.g., the user 308 has indicated a preference for watching the morning news upon getting out of bed). The control circuitry 334 can generate the control signal and transmit the signal to the television 312 to cause the television 312 to turn on and tune to the desired station (which can be stored at the control circuitry 334, the television 312, or another location). As another example, upon detecting that the user 308 has gotten up for the day, the control circuitry 334 can generate and transmit control signals to cause the television 312 to turn on and begin playing a previously recorded program from a digital video recorder (DVR) in communication with the television 312.

As another example, if the television 312 is in the same room as the bed 302, the control circuitry 334 may not cause the television 312 to turn off in response to detection of user bed presence. Rather, the control circuitry 334 can generate and transmit control signals to cause the television 312 to turn off in response to determining that the user 308 is asleep. For example, the control circuitry 334 can monitor biometric signals of the user 308 (e.g., motion, heartrate, respiration rate) to determine that the user 308 has fallen asleep. Upon detecting that the user 308 is sleeping, the control circuitry 334 generates and transmits a control signal to turn the television 312 off. As another example, the control circuitry 334 can generate the control signal to turn off the television 312 after a threshold period of time has passed since the user 308 has fallen asleep (e.g., 10 minutes after the user has fallen asleep). As another example, the control circuitry 334 generates control signals to lower the volume of the television 312 after determining that the user 308 is asleep. As yet another example, the control circuitry 334 generates and transmits a control signal to cause the television to gradually lower in volume over a period of time and then turn off in response to determining that the user 308 is asleep. Any of the control signals described above in reference to the television 312 can also be determined by the central controller previously described.

In some implementations, the control circuitry 334 can similarly interact with other media devices, such as computers, tablets, mobile phones, smart phones, wearable devices, stereo systems, etc. For example, upon detecting that the user 308 is asleep, the control circuitry 334 can generate and transmit a control signal to the user device 310 to cause the user device 310 to turn off, or turn down the volume on a video or audio file being played by the user device 310.

The control circuitry 334 can additionally communicate with the lighting system 314, receive information from the lighting system 314, and generate control signals for controlling functions of the lighting system 314. For example, upon detecting user bed presence on the bed 302 during a certain time frame (e.g., between 8:00 pm and 7:00 am) that lasts for longer than a threshold period of time (e.g., 10 minutes), the control circuitry 334 of the bed 302 can determine that the user 308 is in bed for the evening. In response to this determination, the control circuitry 334 can generate control signals to cause lights in one or more rooms other than the room in which the bed 302 is located to switch off. The control signals can then be transmitted to the lighting system 314 and executed by the lighting system 314 to cause the lights in the indicated rooms to shut off. For example, the control circuitry 334 can generate and transmit control signals to turn off lights in all common rooms, but not in other bedrooms. As another example, the control signals generated by the control circuitry 334 can indicate that lights in all rooms other than the room in which the bed 302 is located are to be turned off, while one or more lights located outside of the house containing the bed 302 are to be turned on, in response to determining that the user 308 is in bed for the evening. Additionally, the control circuitry 334 can generate and transmit control signals to cause the nightlight 328 to turn on in response to determining user 308 bed presence or that the user 308 is asleep. As another example, the control circuitry 334 can generate first control signals for turning off a first set of lights (e.g., lights in common rooms) in response to detecting user bed presence, and second control signals for turning off a second set of lights (e.g., lights in the room in which the bed 302 is located) in response to detecting that the user 308 is asleep.

In some implementations, in response to determining that the user 308 is in bed for the evening, the control circuitry 334 of the bed 302 can generate control signals to cause the lighting system 314 to implement a sunset lighting scheme in the room in which the bed 302 is located. A sunset lighting scheme can include, for example, dimming the lights (either gradually over time, or all at once) in combination with changing the color of the light in the bedroom environment, such as adding an amber hue to the lighting in the bedroom. The sunset lighting scheme can help to put the user 308 to sleep when the control circuitry 334 has determined that the user 308 is in bed for the evening. Sometimes, the control signals can cause the lighting system 314 to dim the lights or change color of the lighting in the bedroom environment, but not both.

The control circuitry 334 can also be configured to implement a sunrise lighting scheme when the user 308 wakes up in the morning. The control circuitry 334 can determine that the user 308 is awake for the day, for example, by detecting that the user 308 has gotten off of the bed 302 (e.g., is no longer present on the bed 302) during a specified time frame (e.g., between 6:00 am and 8:00 am). As another example, the control circuitry 334 can monitor movement, heartrate, respiratory rate, or other biometric signals of the user 308 to determine that the user 308 is awake or is waking up, even though the user 308 has not gotten out of bed. If the control circuitry 334 detects that the user is awake or waking up during a specified timeframe, the control circuitry 334 can determine that the user 308 is awake for the day. The specified timeframe can be, for example, based on previously recorded user bed presence information collected over a period of time (e.g., two weeks) that indicates that the user 308 usually wakes up for the day between 6:30 am and 7:30 am. In response to the control circuitry 334 determining that the user 308 is awake, the control circuitry 334 can generate control signals to cause the lighting system 314 to implement the sunrise lighting scheme in the bedroom in which the bed 302 is located. The sunrise lighting scheme can include, for example, turning on lights (e.g., the lamp 326, or other lights in the bedroom). The sunrise lighting scheme can further include gradually increasing the level of light in the room where the bed 302 is located (or in one or more other rooms). The sunrise lighting scheme can also include only turning on lights of specified colors. For example, the sunrise lighting scheme can include lighting the bedroom with blue light to gently assist the user 308 in waking up and becoming active.

In some implementations, the control circuitry 334 can generate different control signals for controlling actions of one or more components, such as the lighting system 314, depending on a time of day that user interactions with the bed 302 are detected. For example, the control circuitry 334 can use historical user interaction information for interactions between the user 308 and the bed 302 to determine that the user 308 usually falls asleep between 10:00 pm and 11:00 pm and usually wakes up between 6:30 am and 7:30 am on weekdays. The control circuitry 334 can use this information to generate a first set of control signals for controlling the lighting system 314 if the user 308 is detected as getting out of bed at 3:00 am and to generate a second set of control signals for controlling the lighting system 314 if the user 308 is detected as getting out of bed after 6:30 am. For example, if the user 308 gets out of bed prior to 6:30 am, the control circuitry 334 can turn on lights that guide the user 308's route to a bathroom. As another example, if the user 308 gets out of bed prior to 6:30 am, the control circuitry 334 can turn on lights that guide the user 308's route to the kitchen (which can include, for example, turning on the nightlight 328, turning on under bed lighting, turning on the lamp 326, or turning on lights along a path that the user 308 takes to get to the kitchen).

As another example, if the user 308 gets out of bed after 6:30 am, the control circuitry 334 can generate control signals to cause the lighting system 314 to initiate a sunrise lighting scheme, or to turn on one or more lights in the bedroom and/or other rooms. In some implementations, if the user 308 is detected as getting out of bed prior to a specified morning rise time for the user 308, the control circuitry 334 can cause the lighting system 314 to turn on lights that are dimmer than lights that are turned on by the lighting system 314 if the user 308 is detected as getting out of bed after the specified morning rise time. Causing the lighting system 314 to only turn on dim lights when the user 308 gets out of bed during the night (e.g., prior to normal rise time for the user 308) can prevent other occupants of the house from being woken up by the lights while still allowing the user 308 to see in order to reach the bathroom, kitchen, or another destination in the house.

The historical user interaction information for interactions between the user 308 and the bed 302 can be used to identify user sleep and awake timeframes. For example, user bed presence times and sleep times can be determined for a set period of time (e.g., two weeks, a month, etc.). The control circuitry 334 can then identify a typical time range or timeframe in which the user 308 goes to bed, a typical timeframe for when the user 308 falls asleep, and a typical timeframe for when the user 308 wakes up (and in some cases, different timeframes for when the user 308 wakes up and when the user 308 actually gets out of bed). In some implementations, buffer time can be added to these timeframes. For example, if the user is identified as typically going to bed between 10:00 pm and 10:30 pm, a buffer of a half hour in each direction can be added to the timeframe such that any detection of the user getting in bed between 9:30 pm and 11:00 pm is interpreted as the user 308 going to bed for the evening. As another example, detection of bed presence of the user 308 starting from a half hour before the earliest typical time that the user 308 goes to bed extending until the typical wake up time (e.g., 6:30 am) for the user 308 can be interpreted as the user 308 going to bed for the evening. For example, if the user 308 typically goes to bed between 10:00 pm and 10:30 pm, if the user 308's bed presence is sensed at 12:30 am one night, that can be interpreted as the user 308 getting into bed for the evening even though this is outside of the user 308's typical timeframe for going to bed because it has occurred prior to the user 308's normal wake up time. In some implementations, different timeframes are identified for different times of the year (e.g., earlier bed time during winter vs. summer) or at different times of the week (e.g., user 308 wakes up earlier on weekdays than on weekends).

The control circuitry 334 can distinguish between the user 308 going to bed for an extended period (such as for the night) as opposed to being present on the bed 302 for a shorter period (such as for a nap) by sensing duration of presence of the user 308 (e.g., by detecting pressure signals and/or temperature signals of the user 308 on the bed 302 by one or more sensors that are integrated into the bed 302). In some examples, the control circuitry 334 can distinguish between the user 308 going to bed for an extended period (such as for the night) as opposed to going to bed for a shorter period (such as for a nap) by sensing duration of sleep of the user 308. For example, the control circuitry 334 can set a time threshold whereby if the user 308 is sensed on the bed 302 for longer than the threshold, the user 308 is considered to have gone to bed for the night. In some examples, the threshold can be about 2 hours, whereby if the user 308 is sensed on the bed 302 for greater than 2 hours, the control circuitry 334 registers that as an extended sleep event. In other examples, the threshold can be greater than or less than two hours. The threshold can also be determined based on historic trends indicating how long the user 302 usually sleeps or otherwise stays on the bed 302.

The control circuitry 334 can detect repeated extended sleep events to automatically determine a typical bed time range of the user 308, without requiring the user 308 to enter a bed time range. This can allow the control circuitry 334 to accurately estimate when the user 308 is likely to go to bed for an extended sleep event, regardless of whether the user 308 typically goes to bed using a traditional sleep schedule or a non-traditional sleep schedule. The control circuitry 334 can then use knowledge of the bed time range of the user 308 to control one or more components (including components of the bed 302 and/or non-bed peripherals) based on sensing bed presence during the bed time range or outside of the bed time range.

In some examples, the control circuitry 334 can automatically determine the bed time range of the user 308 without requiring user inputs. In some examples, the control circuitry 334 can determine the bed time range of the user 308 automatically and in combination with user inputs (e.g., using one or more signals that are sensed by sensors of the bed 302 and/or the central controller described above). In some examples, the control circuitry 334 can set the bed time range directly according to user inputs. In some examples, the control circuitry 334 can associate different bed times with different days of the week. In each of these examples, the control circuitry 334 can control one or more components (such as the lighting system 314, the thermostat 316, the security system 318, the oven 322, the coffee maker 324, the lamp 326, and the nightlight 328), as a function of sensed bed presence and the bed time range.

The control circuitry 334 can additionally communicate with the thermostat 316, receive information from the thermostat 316, and generate control signals for controlling functions of the thermostat 316. For example, the user 308 can indicate user preferences for different temperatures at different times, depending on the sleep state or bed presence of the user 308. For example, the user 308 may prefer an environmental temperature of 72 degrees when out of bed, 70 degrees when in bed but awake, and 68 degrees when sleeping. The control circuitry 334 of the bed 302 can detect bed presence of the user 308 in the evening and determine that the user 308 is in bed for the night. In response to this determination, the control circuitry 334 can generate control signals to cause the thermostat 316 to change the temperature to 70 degrees. The control circuitry 334 can then transmit the control signals to the thermostat 316. Upon detecting that the user 308 is in bed during the bed time range or asleep, the control circuitry 334 can generate and transmit control signals to cause the thermostat 316 to change the temperature to 68. The next morning, upon determining that the user 308 is awake for the day (e.g., the user 308 gets out of bed after 6:30 am), the control circuitry 334 can generate and transmit control circuitry 334 to cause the thermostat to change the temperature to 72 degrees.

The control circuitry 334 can also determine control signals to be transmitted to the thermostat 316 based on maintaining improved or preferred sleep quality of the user 308. In other words, the control circuitry 334 can determine adjustments to the thermostat 316 that are not merely based on user-inputted preferences. For example, the control circuitry 334 can determine, based on historic sleep patterns and quality of the user 308 and by applying one or more machine learning models, that the user 308 experiences their best sleep when the bedroom is at 74 degrees. The control circuitry 334 can receive temperature signals from one or more devices and/or sensors in the bedroom indicating a temperature of the bedroom. When the temperature is below 74 degrees, the control circuitry 334 can determine control signals that cause the thermostat 316 to activate a heating unit in the house to raise the temperature to 74 degrees in the bedroom. When the temperature is above 74 degrees, the control circuitry 334 can determine control signals that cause the thermostat 316 to activate a cooling unit in the house to lower the temperature back to 74 degrees. Sometimes, the control circuitry 334 can also determine control signals that cause the thermostat 316 to maintain the bedroom within a temperature range that is intended to keep the user 308 in particular sleep states and/or transition to next preferred sleep states.

In some implementations, the control circuitry 334 can generate control signals to cause one or more heating or cooling elements on the surface of the bed 302 to change temperature at various times, either in response to user interaction with the bed 302, at various pre-programmed times, based on user preference, and/or in response to detecting microclimate temperatures of the user 308 on the bed 302. For example, the control circuitry 334 can activate a heating element to raise the temperature of one side of the surface of the bed 302 to 73 degrees when it is detected that the user 308 has fallen asleep. As another example, upon determining that the user 308 is up for the day, the control circuitry 334 can turn off a heating or cooling element. As yet another example, the user 308 can pre-program various times at which the temperature at the surface of the bed should be raised or lowered. For example, the user 308 can program the bed 302 to raise the surface temperature to 76 degrees at 10:00 pm, and lower the surface temperature to 68 degrees at 11:30 pm. As another example, one or more temperature sensors on the surface of the bed 302 can detect microclimates of the user 308 on the bed 302. When a detected microclimate of the user 308 drops below a predetermined threshold temperature, the control circuitry 334 can activate a heating element to raise the user 308's body temperature, thereby improving the user 308's comfortability, maintaining the user 308 in their sleep cycle, transitioning the user 308 to a next preferred sleep state, and/or otherwise maintaining or improving the user 308's sleep quality.

In some implementations, in response to detecting user bed presence of the user 308 and/or that the user 308 is asleep, the control circuitry 334 can cause the thermostat 316 to change the temperature in different rooms to different values. For example, in response to determining that the user 308 is in bed for the evening, the control circuitry 334 can generate and transmit control signals to cause the thermostat 316 to set the temperature in one or more bedrooms of the house to 72 degrees and set the temperature in other rooms to 67 degrees. Other control signals are also possible, and can be based on user preference and user input.

The control circuitry 334 can also receive temperature information from the thermostat 316 and use this temperature information to control functions of the bed 302 or other devices. For example, as discussed above, the control circuitry 334 can adjust temperatures of heating elements included in or otherwise attached to the bed 302 (e.g., a foot warming pad) in response to temperature information received from the thermostat 316.

In some implementations, the control circuitry 334 can generate and transmit control signals for controlling other temperature control systems. For example, in response to determining that the user 308 is awake for the day, the control circuitry 334 can generate and transmit control signals for causing floor heating elements to activate in the bedroom and/or in other rooms in the house. For example, the control circuitry 334 can cause a floor heating system in a master bedroom to turn on in response to determining that the user 308 is awake for the day. One or more of the control signals described herein that are determined by the control circuitry 334 can also be determined by the central controller described above.

The control circuitry 334 can additionally communicate with the security system 318, receive information from the security system 318, and generate control signals for controlling functions of the security system 318. For example, in response to detecting that the user 308 in is bed for the evening, the control circuitry 334 can generate control signals to cause the security system 318 to engage or disengage security functions. The control circuitry 334 can then transmit the control signals to the security system 318 to cause the security system 318 to engage (e.g., turning on security cameras along a perimeter of the house, automatically locking doors in the house, etc.). As another example, the control circuitry 334 can generate and transmit control signals to cause the security system 318 to disable in response to determining that the user 308 is awake for the day (e.g., user 308 is no longer present on the bed 302 after 6:00 am). In some implementations, the control circuitry 334 can generate and transmit a first set of control signals to cause the security system 318 to engage a first set of security features in response to detecting user bed presence of the user 308, and can generate and transmit a second set of control signals to cause the security system 318 to engage a second set of security features in response to detecting that the user 308 has fallen asleep.

In some implementations, the control circuitry 334 can receive alerts from the security system 318 and indicate the alert to the user 308. For example, the control circuitry 334 can detect that the user 308 is in bed for the evening and in response, generate and transmit control signals to cause the security system 318 to engage or disengage. The security system can then detect a security breach (e.g., someone has opened the door 332 without entering the security code, or someone has opened a window when the security system 318 is engaged). The security system 318 can communicate the security breach to the control circuitry 334 of the bed 302. In response to receiving the communication from the security system 318, the control circuitry 334 can generate control signals to alert the user 308 to the security breach. For example, the control circuitry 334 can cause the bed 302 to vibrate. As another example, the control circuitry 334 can cause portions of the bed 302 to articulate (e.g., cause the head section to raise or lower) in order to wake the user 308 and alert the user to the security breach. As another example, the control circuitry 334 can generate and transmit control signals to cause the lamp 326 to flash on and off at regular intervals to alert the user 308 to the security breach. As another example, the control circuitry 334 can alert the user 308 of one bed 302 regarding a security breach in a bedroom of another bed, such as an open window in a kid's bedroom. As another example, the control circuitry 334 can send an alert to a garage door controller (e.g., to close and lock the door). As another example, the control circuitry 334 can send an alert for the security to be disengaged. The control circuitry 334 can also set off a smart alarm or other alarm device/clock near the bed 302. The control circuitry 334 can transmit a push notification, text message, or other indication of the security breach to the user device 310. Also, the control circuitry 334 can transmit a notification of the security breach to the central controller described above The central controller can then determine one or more responses to the security breach.

The control circuitry 334 can additionally generate and transmit control signals for controlling the garage door 320 and receive information indicating a state of the garage door 320 (e.g., open or closed). For example, in response to determining that the user 308 is in bed for the evening, the control circuitry 334 can generate and transmit a request to a garage door opener or another device capable of sensing if the garage door 320 is open. The control circuitry 334 can request information on the current state of the garage door 320. If the control circuitry 334 receives a response (e.g., from the garage door opener) indicating that the garage door 320 is open, the control circuitry 334 can either notify the user 308 that the garage door is open (e.g., by displaying a notification or other message at the user device 310, by outputting a notification at the central controller, etc.), and/or generate a control signal to cause the garage door opener to close the garage door 320. For example, the control circuitry 334 can send a message to the user device 310 indicating that the garage door is open. As another example, the control circuitry 334 can cause the bed 302 to vibrate. As yet another example, the control circuitry 334 can generate and transmit a control signal to cause the lighting system 314 to cause one or more lights in the bedroom to flash to alert the user 308 to check the user device 310 for an alert (in this example, an alert regarding the garage door 320 being open). Alternatively, or additionally, the control circuitry 334 can generate and transmit control signals to cause the garage door opener to close the garage door 320 in response to identifying that the user 308 is in bed for the evening and that the garage door 320 is open. Control signals can also vary depend on the age of the user 308.

The control circuitry 334 can similarly send and receive communications for controlling or receiving state information associated with the door 332 or the oven 322. For example, upon detecting that the user 308 is in bed for the evening, the control circuitry 334 can generate and transmit a request to a device or system for detecting a state of the door 332. Information returned in response to the request can indicate various states of the door 332 such as open, closed but unlocked, or closed and locked. If the door 332 is open or closed but unlocked, the control circuitry 334 can alert the user 308 to the state of the door, such as in a manner described above with reference to the garage door 320. Alternatively, or in addition to alerting the user 308, the control circuitry 334 can generate and transmit control signals to cause the door 332 to lock, or to close and lock. If the door 332 is closed and locked, the control circuitry 334 can determine that no further action is needed.

Similarly, upon detecting that the user 308 is in bed for the evening, the control circuitry 334 can generate and transmit a request to the oven 322 to request a state of the oven 322 (e.g., on or off). If the oven 322 is on, the control circuitry 334 can alert the user 308 and/or generate and transmit control signals to cause the oven 322 to turn off. If the oven is already off, the control circuitry 334 can determine that no further action is necessary. In some implementations, different alerts can be generated for different events. For example, the control circuitry 334 can cause the lamp 326 (or one or more other lights, via the lighting system 314) to flash in a first pattern if the security system 318 has detected a breach, flash in a second pattern if garage door 320 is on, flash in a third pattern if the door 332 is open, flash in a fourth pattern if the oven 322 is on, and flash in a fifth pattern if another bed has detected that a user 308 of that bed has gotten up (e.g., that a child of the user 308 has gotten out of bed in the middle of the night as sensed by a sensor in the child's bed). Other examples of alerts that can be processed by the control circuitry 334 of the bed 302 and communicated to the user (e.g., at the user device 310 and/or the central controller described herein) include a smoke detector detecting smoke (and communicating this detection of smoke to the control circuitry 334), a carbon monoxide tester detecting carbon monoxide, a heater malfunctioning, or an alert from any other device capable of communicating with the control circuitry 334 and detecting an occurrence that should be brought to the user 308's attention.

The control circuitry 334 can also communicate with a system or device for controlling a state of the window blinds 330. For example, in response to determining that the user 308 is in bed for the evening, the control circuitry 334 can generate and transmit control signals to cause the window blinds 330 to close. As another example, in response to determining that the user 308 is up for the day (e.g., user has gotten out of bed after 6:30 am) or that the user 308 set an alarm to wake up at a particular time, the control circuitry 334 can generate and transmit control signals to cause the window blinds 330 to open. By contrast, if the user 308 gets out of bed prior to a normal rise time for the user 308, the control circuitry 334 can determine that the user 308 is not awake for the day and may not generate control signals that cause the window blinds 330 to open. As yet another example, the control circuitry 334 can generate and transmit control signals that cause a first set of blinds to close in response to detecting user bed presence of the user 308 and a second set of blinds to close in response to detecting that the user 308 is asleep.

The control circuitry 334 can generate and transmit control signals for controlling functions of other household devices in response to detecting user interactions with the bed 302. For example, in response to determining that the user 308 is awake for the day, the control circuitry 334 can generate and transmit control signals to the coffee maker 324 to cause the coffee maker 324 to begin brewing coffee. As another example, the control circuitry 334 can generate and transmit control signals to the oven 322 to cause the oven 322 to begin preheating (for users that like fresh baked bread in the morning or otherwise bake or prepare food in the morning). As another example, the control circuitry 334 can use information indicating that the user 308 is awake for the day along with information indicating that the time of year is currently winter and/or that the outside temperature is below a threshold value to generate and transmit control signals to cause a car engine block heater to turn on.

As another example, the control circuitry 334 can generate and transmit control signals to cause one or more devices to enter a sleep mode in response to detecting user bed presence of the user 308, or in response to detecting that the user 308 is asleep. For example, the control circuitry 334 can generate control signals to cause a mobile phone of the user 308 to switch into sleep mode or night mode such that notifications from the mobile phone are muted to not disturb the user 308's sleep. The control circuitry 334 can then transmit the control signals to the mobile phone. Later, upon determining that the user 308 is up for the day, the control circuitry 334 can generate and transmit control signals to cause the mobile phone to switch out of sleep mode.

In some implementations, the control circuitry 334 can communicate with one or more noise control devices. For example, upon determining that the user 308 is in bed for the evening, or that the user 308 is asleep (e.g., based on pressure signals received from the bed 302, audio/decibel signals received from audio sensors positioned on or around the bed 302, etc.), the control circuitry 334 can generate and transmit control signals to cause one or more noise cancelation devices to activate. The noise cancelation devices can, for example, be included as part of the bed 302 or located in the bedroom with the bed 302. As another example, upon determining that the user 308 is in bed for the evening or that the user 308 is asleep, the control circuitry 334 can generate and transmit control signals to turn the volume on, off, up, or down, for one or more sound generating devices, such as a stereo system radio, television, computer, tablet, mobile phone, etc.

Additionally, functions of the bed 302 can be controlled by the control circuitry 334 in response to user interactions with the bed 302. As mentioned throughout, functions of the bed 302 described herein can also be controlled by the user device 310 and/or the central controller (e.g., a hub device or other home automation device that controls multiple different devices in the home). As mentioned above, the bed 302 can include an adjustable foundation and an articulation controller configured to adjust the position of one or more portions of the bed 302 by adjusting the adjustable foundation that supports the bed 302. For example, the articulation controller can adjust the bed 302 from a flat position to a position in which a head portion of a mattress of the bed 302 is inclined upward (e.g., to facilitate a user sitting up in bed, reading, and/or watching television). In some implementations, the bed 302 includes multiple separately articulable sections. For example, portions of the bed corresponding to the locations of the air chambers 306a and 306b can be articulated independently from each other, to allow one person positioned on the bed 302 surface to rest in a first position (e.g., a flat position) while a second person rests in a second position (e.g., a reclining position with the head raised at an angle from the waist). In some implementations, separate positions can be set for two different beds (e.g., two twin beds placed next to each other). The foundation of the bed 302 can include more than one zone that can be independently adjusted. The articulation controller can also be configured to provide different levels of massage to one or more users on the bed 302 or to cause the bed to vibrate to communicate alerts to the user 308 as described above.

The control circuitry 334 can adjust positions (e.g., incline and decline positions for the user 308 and/or an additional user of the bed 302) in response to user interactions with the bed 302. For example, the control circuitry 334 can cause the articulation controller to adjust the bed 302 to a first recline position for the user 308 in response to sensing user bed presence for the user 308. The control circuitry 334 can cause the articulation controller to adjust the bed 302 to a second recline position (e.g., a less reclined, or flat position) in response to determining that the user 308 is asleep. As another example, the control circuitry 334 can receive a communication from the television 312 indicating that the user 308 has turned off the television 312, and in response, the control circuitry 334 can cause the articulation controller to adjust the position of the bed 302 to a preferred user sleeping position (e.g., due to the user turning off the television 312 while the user 308 is in bed indicating that the user 308 wishes to go to sleep).

In some implementations, the control circuitry 334 can control the articulation controller so as to wake up one user of the bed 302 without waking another user of the bed 302. For example, the user 308 and a second user of the bed 302 can each set distinct wakeup times (e.g., 6:30 am and 7:15 am respectively). When the wakeup time for the user 308 is reached, the control circuitry 334 can cause the articulation controller to vibrate or change the position of only a side of the bed on which the user 308 is located to wake the user 308 without disturbing the second user. When the wakeup time for the second user is reached, the control circuitry 334 can cause the articulation controller to vibrate or change the position of only the side of the bed on which the second user is located. Alternatively, when the second wakeup time occurs, the control circuitry 334 can utilize other methods (such as audio alarms, or turning on the lights) to wake the second user since the user 308 is already awake and therefore will not be disturbed when the control circuitry 334 attempts to wake the second user.

Still referring to FIG. 3, the control circuitry 334 for the bed 302 can utilize information for interactions with the bed 302 by multiple users to generate control signals for controlling functions of various other devices. For example, the control circuitry 334 can wait to generate control signals for, for example, engaging the security system 318, or instructing the lighting system 314 to turn off lights in various rooms, until both the user 308 and a second user are detected as being present on the bed 302. As another example, the control circuitry 334 can generate a first set of control signals to cause the lighting system 314 to turn off a first set of lights upon detecting bed presence of the user 308 and generate a second set of control signals for turning off a second set of lights in response to detecting bed presence of a second user. As another example, the control circuitry 334 can wait until it has been determined that both the user 308 and a second user are awake for the day before generating control signals to open the window blinds 330. As yet another example, in response to determining that the user 308 has left the bed 302 and is awake for the day, but that a second user is still sleeping, the control circuitry 334 can generate and transmit a first set of control signals to cause the coffee maker 324 to begin brewing coffee, to cause the security system 318 to deactivate, to turn on the lamp 326, to turn off the nightlight 328, to cause the thermostat 316 to raise the temperature in one or more rooms to 72 degrees, and/or to open the window blinds 330 in rooms other than the bedroom in which the bed 302 is located. Later, in response to detecting that the second user is no longer present on the bed (or that the second user is awake or is waking up) the control circuitry 334 can generate and transmit a second set of control signals to, for example, cause the lighting system 314 to turn on one or more lights in the bedroom, to cause window blinds in the bedroom to open, and to turn on the television 312 to a pre-specified channel. One or more other home automation control signals can be determined and generated by the control circuitry 334, the user device 310, and/or the central controller described herein.

Examples of Data Processing Systems Associated with a Bed

Described here are examples of systems and components that can be used for data processing tasks that are, for example, associated with a bed. In some cases, multiple examples of a particular component or group of components are presented. Some of these examples are redundant and/or mutually exclusive alternatives. Connections between components are shown as examples to illustrate possible network configurations for allowing communication between components. Different formats of connections can be used as technically needed or desired. The connections generally indicate a logical connection that can be created with any technologically feasible format. For example, a network on a motherboard can be created with a printed circuit board, wireless data connections, and/or other types of network connections. Some logical connections are not shown for clarity. For example, connections with power supplies and/or computer readable memory may not be shown for clarities sake, as many or all elements of a particular component may need to be connected to the power supplies and/or computer readable memory.

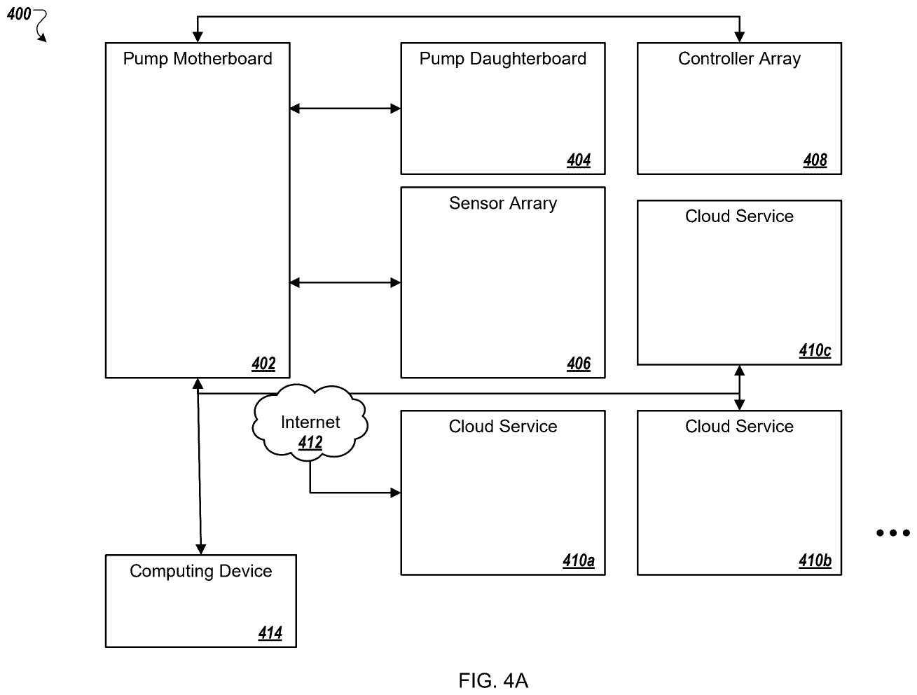

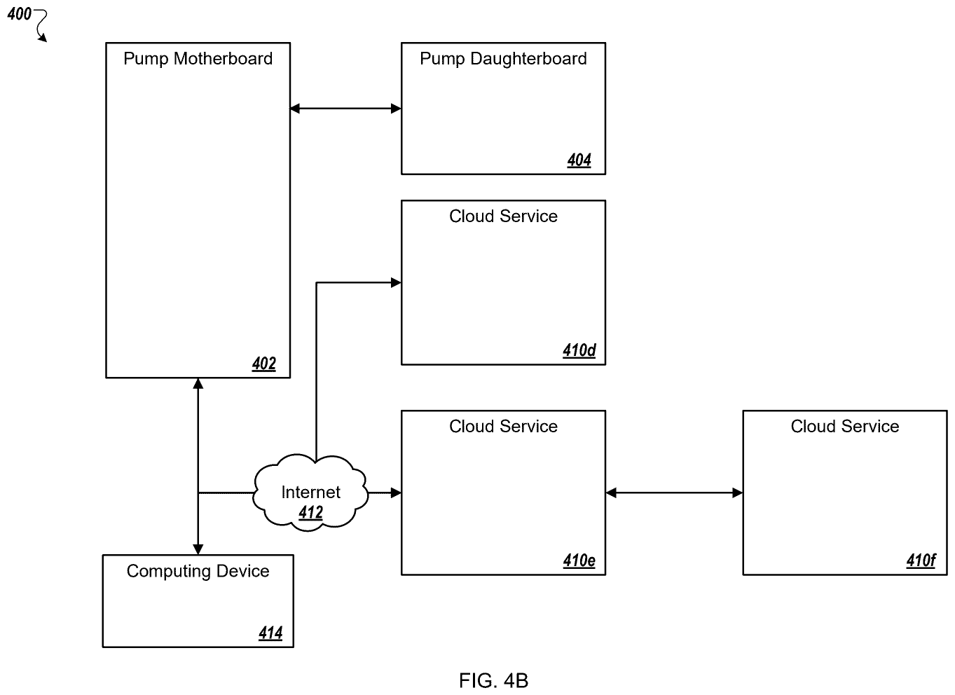

FIG. 4A is a block diagram of an example of a data processing system 400 that can be associated with a bed system, including those described above with respect to FIGS. 1-3. This system 400 includes a pump motherboard 402 and a pump daughterboard 404. The system 400 includes a sensor array 406 that can include one or more sensors configured to sense physical phenomenon of the environment and/or bed, and to report such sensing back to the pump motherboard 402 for, for example, analysis. The sensor array 406 can include one or more different types of sensors, including but not limited to pressure sensors, temperature sensors, light sensors, movement (e.g. motion) sensors, and audio sensors. The system 400 also includes a controller array 408 that can include one or more controllers configured to control logic-controlled devices of the bed and/or environment (such as home automation devices, security systems light systems, and other devices that are described in reference to FIG. 3). The pump motherboard 400 can be in communication with one or more computing devices 414 and one or more cloud services 410 over local networks, the Internet 412, or otherwise as is technically appropriate. Each of these components will be described in more detail, some with multiple example configurations, below.

In this example, a pump motherboard 402 and a pump daughterboard 404 are communicably coupled. They can be conceptually described as a center or hub of the system 400, with the other components conceptually described as spokes of the system 400. In some configurations, this can mean that each of the spoke components communicates primarily or exclusively with the pump motherboard 402. For example, a sensor of the sensor array 406 may not be configured to, or may not be able to, communicate directly with a corresponding controller. Instead, each spoke component can communicate with the motherboard 402. The sensor of the sensor array 406 can report a sensor reading to the motherboard 402, and the motherboard 402 can determine that, in response, a controller of the controller array 408 should adjust some parameters of a logic controlled device or otherwise modify a state of one or more peripheral devices. In one case, if the temperature of the bed is determined to be too hot based on received temperature signals from the sensor array 406, the pump motherboard 402 can determine that a temperature controller should cool the bed.

One advantage of a hub-and-spoke network configuration, sometimes also referred to as a star-shaped network, is a reduction in network traffic compared to, for example, a mesh network with dynamic routing. If a particular sensor generates a large, continuous stream of traffic, that traffic may only be transmitted over one spoke of the network to the motherboard 402. The motherboard 402 can, for example, marshal that data and condense it to a smaller data format for retransmission for storage in a cloud service 410. Additionally or alternatively, the motherboard 402 can generate a single, small, command message to be sent down a different spoke of the network in response to the large stream. For example, if the large stream of data is a pressure reading that is transmitted from the sensor array 406 a few times a second, the motherboard 402 can respond with a single command message to the controller array to increase the pressure in an air chamber of the bed. In this case, the single command message can be orders of magnitude smaller than the stream of pressure readings.

As another advantage, a hub-and-spoke network configuration can allow for an extensible network that can accommodate components being added, removed, failing, etc. This can allow, for example, more, fewer, or different sensors in the sensor array 406, controllers in the controller array 408, computing devices 414, and/or cloud services 410. For example, if a particular sensor fails or is deprecated by a newer version of the sensor, the system 400 can be configured such that only the motherboard 402 needs to be updated about the replacement sensor. This can allow, for example, product differentiation where the same motherboard 402 can support an entry level product with fewer sensors and controllers, a higher value product with more sensors and controllers, and customer personalization where a customer can add their own selected components to the system 400.

Additionally, a line of air bed products can use the system 400 with different components. In an application in which every air bed in the product line includes both a central logic unit and a pump, the motherboard 402 (and optionally the daughterboard 404) can be designed to fit within a single, universal housing. Then, for each upgrade of the product in the product line, additional sensors, controllers, cloud services, etc., can be added. Design, manufacturing, and testing time can be reduced by designing all products in a product line from this base, compared to a product line in which each product has a bespoke logic control system.

Each of the components discussed above can be realized in a wide variety of technologies and configurations. Below, some examples of each component will be further discussed. In some alternatives, two or more of the components of the system 400 can be realized in a single alternative component; some components can be realized in multiple, separate components; and/or some functionality can be provided by different components.

FIG. 4B is a block diagram showing some communication paths of the data processing system 400. As previously described, the motherboard 402 and the pump daughterboard 404 may act as a hub for peripheral devices and cloud services of the system 400. In cases in which the pump daughterboard 404 communicates with cloud services or other components, communications from the pump daughterboard 404 may be routed through the pump motherboard 402. This may allow, for example, the bed to have only a single connection with the internet 412. The computing device 414 may also have a connection to the internet 412, possibly through the same gateway used by the bed and/or possibly through a different gateway (e.g., a cell service provider).

Previously, a number of cloud services 410 were described. As shown in FIG. 4B, some cloud services, such as cloud services 410d and 410e, may be configured such that the pump motherboard 402 can communicate with the cloud service directly—that is the motherboard 402 may communicate with a cloud service 410 without having to use another cloud service 410 as an intermediary. Additionally or alternatively, some cloud services 410, for example cloud service 410f, may only be reachable by the pump motherboard 402 through an intermediary cloud service, for example cloud service 410e. While not shown here, some cloud services 410 may be reachable either directly or indirectly by the pump motherboard 402.

Additionally, some or all of the cloud services 410 may be configured to communicate with other cloud services. This communication may include the transfer of data and/or remote function calls according to any technologically appropriate format. For example, one cloud service 410 may request a copy for another cloud service's 410 data, for example, for purposes of backup, coordination, migration, or for performance of calculations or data mining. In another example, many cloud services 410 may contain data that is indexed according to specific users tracked by the user account cloud 410c and/or the bed data cloud 410a. These cloud services 410 may communicate with the user account cloud 410c and/or the bed data cloud 410a when accessing data specific to a particular user or bed.

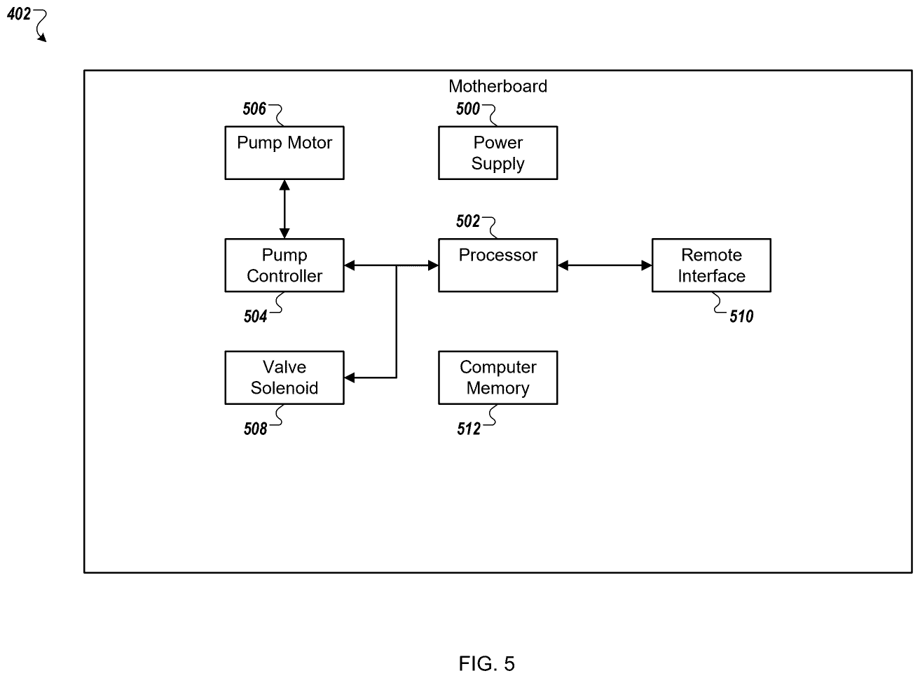

FIG. 5 is a block diagram of an example of a motherboard 402 that can be used in a data processing system that can be associated with a bed system, including those described above with respect to FIGS. 1-3. In this example, compared to other examples described below, this motherboard 402 consists of relatively fewer parts and can be limited to provide a relatively limited feature set.

The motherboard 402 includes a power supply 500, a processor 502, and computer memory 512. In general, the power supply 500 includes hardware used to receive electrical power from an outside source and supply it to components of the motherboard 402. The power supply can include, for example, a battery pack and/or wall outlet adapter, an AC to DC converter, a DC to AC converter, a power conditioner, a capacitor bank, and/or one or more interfaces for providing power in the current type, voltage, etc., needed by other components of the motherboard 402.

The processor 502 is generally a device for receiving input, performing logical determinations, and providing output. The processor 502 can be a central processing unit, a microprocessor, general purpose logic circuitry, application-specific integrated circuitry, a combination of these, and/or other hardware for performing the functionality needed.

The memory 512 is generally one or more devices for storing data. The memory 512 can include long term stable data storage (e.g., on a hard disk), short term unstable (e.g., on Random Access Memory) or any other technologically appropriate configuration.

The motherboard 402 includes a pump controller 504 and a pump motor 506. The pump controller 504 can receive commands from the processor 502 and, in response, control the functioning of the pump motor 506. For example, the pump controller 504 can receive, from the processor 502, a command to increase pressure of an air chamber by 0.3 pounds per square inch (PSI). The pump controller 504, in response, engages a valve so that the pump motor 506 is configured to pump air into the selected air chamber, and can engage the pump motor 506 for a length of time that corresponds to 0.3 PSI or until a sensor indicates that pressure has been increased by 0.3 PSI. In an alternative configuration, the message can specify that the chamber should be inflated to a target PSI, and the pump controller 504 can engage the pump motor 506 until the target PSI is reached.

A valve solenoid 508 can control which air chamber a pump is connected to. In some cases, the solenoid 508 can be controlled by the processor 502 directly. In some cases, the solenoid 508 can be controlled by the pump controller 504.

A remote interface 510 of the motherboard 402 can allow the motherboard 402 to communicate with other components of a data processing system. For example, the motherboard 402 can be able to communicate with one or more daughterboards, with peripheral sensors, and/or with peripheral controllers through the remote interface 510. The remote interface 510 can provide any technologically appropriate communication interface, including but not limited to multiple communication interfaces such as WIFI, Bluetooth, and copper wired networks.

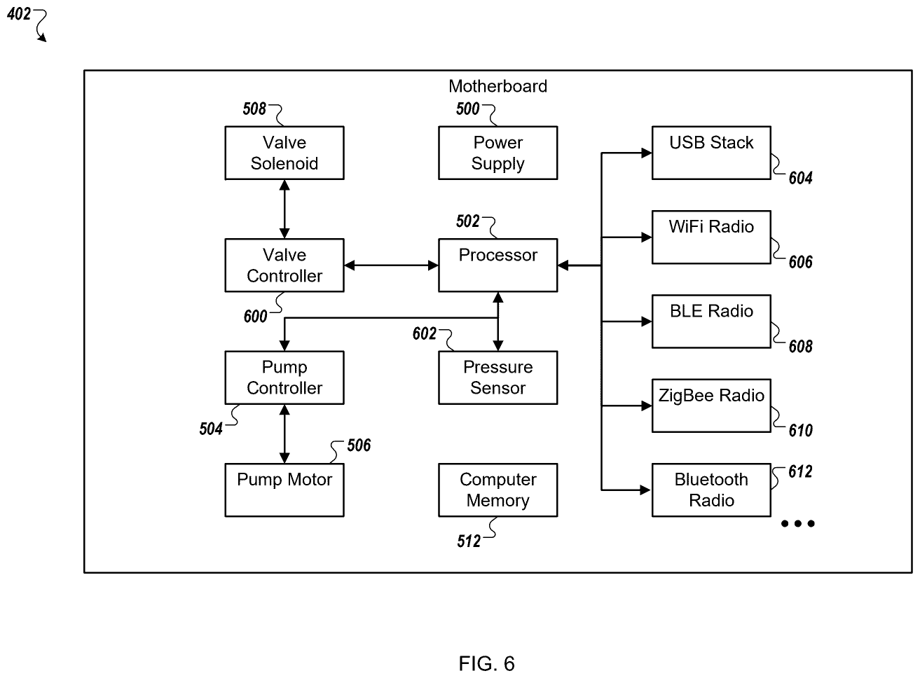

FIG. 6 is a block diagram of an example of the motherboard 402 that can be used in a data processing system associated with a bed system, including those described above with respect to FIGS. 1-3. Compared to the motherboard 402 described with reference to FIG. 5, the motherboard 402 in FIG. 6 can contain more components and provide more functionality in some applications.

In addition to the power supply 500, processor 502, pump controller 504, pump motor 506, and valve solenoid 508, this motherboard 402 is shown with a valve controller 600, a pressure sensor 602, a universal serial bus (USB) stack 604, a WiFi radio 606, a Bluetooth Low Energy (BLE) radio 608, a ZigBee radio 610, a Bluetooth radio 612, and a computer memory 512.

Similar to the way that the pump controller 504 converts commands from the processor 502 into control signals for the pump motor 506, the valve controller 600 can convert commands from the processor 502 into control signals for the valve solenoid 508. In one example, the processor 502 can issue a command to the valve controller 600 to connect the pump to a particular air chamber out of a group of air chambers in an air bed. The valve controller 600 can control the position of the valve solenoid 508 so that the pump is connected to the indicated air chamber.

The pressure sensor 602 can read pressure readings from one or more air chambers of the air bed. The pressure sensor 602 can also preform digital sensor conditioning. As described herein, multiple pressure sensors 602 can be included as part of the motherboard 402 or otherwise in communication with the motherboard 402.

The motherboard 402 can include a suite of network interfaces 604, 606, 608, 610, 612, etc., including but not limited to those shown in FIG. 6. These network interfaces can allow the motherboard to communicate over a wired or wireless network with any number of devices, including but not limited to peripheral sensors, peripheral controllers, computing devices, and devices and services connected to the Internet 412.

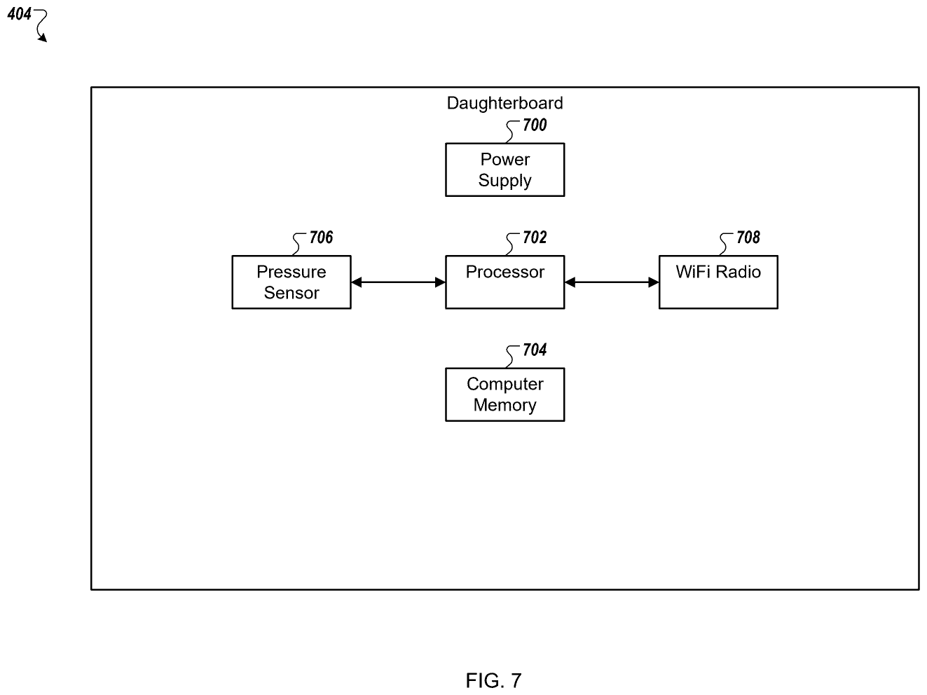

FIG. 7 is a block diagram of an example of a daughterboard 404 that can be used in a data processing system associated with a bed system, including those described above with respect to FIGS. 1-3. In some configurations, one or more daughterboards 404 can be connected to the motherboard 402. Some daughterboards 404 can be designed to offload particular and/or compartmentalized tasks from the motherboard 402. This can be advantageous, for example, if the particular tasks are computationally intensive, proprietary, or subject to future revisions. For example, the daughterboard 404 can be used to calculate a particular sleep data metric. This metric can be computationally intensive, and calculating the sleep metric on the daughterboard 404 can free up the resources of the motherboard 402 while the metric is being calculated. Additionally and/or alternatively, the sleep metric can be subject to future revisions. To update the system 400 with the new sleep metric, it is possible that only the daughterboard 404 that calculates that metric need be replaced. In this case, the same motherboard 402 and other components can be used, saving the need to perform unit testing of additional components instead of just the daughterboard 404.

The daughterboard 404 is shown with a power supply 700, a processor 702, computer readable memory 704, a pressure sensor 706, and a WiFi radio 708. The processor 702 can use the pressure sensor 706 to gather information about the pressure of an air chamber or chambers of an air bed. From this data, the processor 702 can perform an algorithm to calculate a sleep metric (e.g., sleep quality, whether a user is presently in the bed, whether the user has fallen asleep, a heartrate of the user, a respiration rate of the user, movement of the user, etc.). In some examples, the sleep metric can be calculated from only the pressure of air chambers. In other examples, the sleep metric can be calculated using signals from a variety of sensors (e.g., a movement sensor, a pressure sensor, a temperature sensor, and/or an audio sensor). In an example in which different data is needed, the processor 702 can receive that data from an appropriate sensor or sensors. These sensors can be internal to the daughterboard 404, accessible via the WiFi radio 708, or otherwise in communication with the processor 702. Once the sleep metric is calculated, the processor 702 can report that sleep metric to, for example, the motherboard 402. The motherboard 402 can then generate instructions for outputting the sleep metric to the user or otherwise using the sleep metric to determine one or more other information about the user or controls to control the bed system and/or peripheral devices.

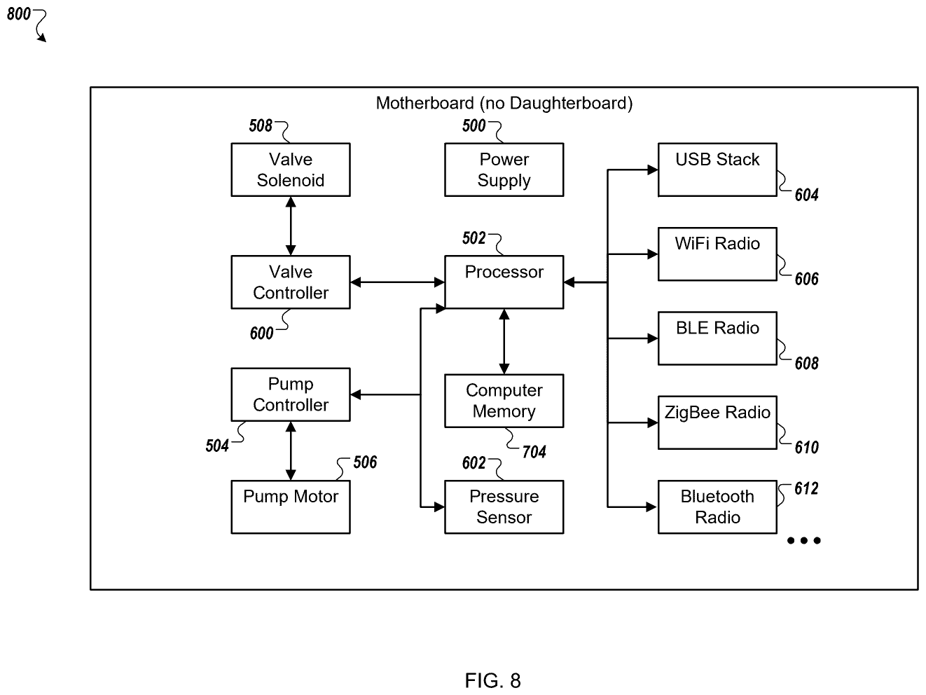

FIG. 8 is a block diagram of an example of a motherboard 800 with no daughterboard that can be used in a data processing system associated with a bed system, including those described above with respect to FIGS. 1-3. In this example, the motherboard 800 can perform most, all, or more of the features described with reference to the motherboard 402 in FIG. 6 and the daughterboard 404 in FIG. 7.

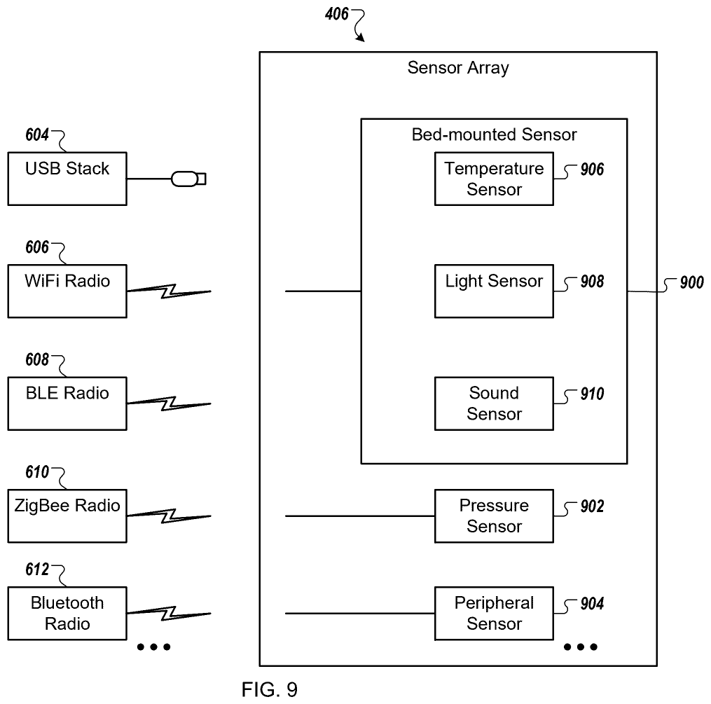

FIG. 9 is a block diagram of an example of the sensory array 406 that can be used in a data processing system associated with a bed system, including those described above with respect to FIGS. 1-3. In general, the sensor array 406 is a conceptual grouping of some or all the peripheral sensors that communicate with the motherboard 402 but are not native to the motherboard 402.

The peripheral sensors 902, 904, 906, 908, 910, etc. of the sensor array 406 can communicate with the motherboard 402 through one or more of the network interfaces of the motherboard, including but not limited to the USB stack 604, WiFi radio 606, Bluetooth Low Energy (BLE) radio 608, ZigBee radio 610, and Bluetooth radio 612, as is appropriate for the configuration of the particular sensor. For example, a sensor that outputs a reading over a USB cable can communicate through the USB stack 604.

Some of the peripheral sensors of the sensor array 406 can be bed mounted sensors 900, such as a temperature sensor 906, a light sensor 908, and a sound sensor 910. The bed mounted sensors 900 can be, for example, embedded into the structure of a bed and sold with the bed, or later affixed to the structure of the bed (e.g., part of a pressure sensing pad that is removably installed on a top surface of the bed, part of a temperature sensing or heating pad that is removably installed on the top surface of the bed, integrated into the top surface of the bed, attached along connecting tubes between a pump and air chambers, within air chambers, attached to a headboard of the bed, attached to one or more regions of an adjustable foundation, etc.). Other sensors 902 and 904 can be in communication with the motherboard 402, but optionally not mounted to the bed. The other sensors 902 and 904 can include a pressure sensor 902 and/or peripheral sensor 904. For example, the sensors 902 and 904 can be integrated or otherwise part of a user mobile device (e.g., mobile phone, wearable device, etc.). The sensors 902 and 904 can also be part of a central controller for controlling the bed and peripheral devices in the home. Sometimes, the sensors 902 and 904 can also be part of one or more home automation devices or other peripheral devices in the home.

In some cases, some or all of the bed mounted sensors 900 and/or sensors 902 and 904 can share networking hardware, including a conduit that contains wires from each sensor, a multi-wire cable or plug that, when affixed to the motherboard 402, connect all of the associated sensors with the motherboard 402. In some embodiments, one, some, or all of sensors 902, 904, 906, 908, and 910 can sense one or more features of a mattress, such as pressure, temperature, light, sound, and/or one or more other features of the mattress. In some embodiments, one, some, or all of sensors 902, 904, 906, 908, and 910 can sense one or more features external to the mattress. In some embodiments, pressure sensor 902 can sense pressure of the mattress while some or all of sensors 902, 904, 906, 908, and 910 can sense one or more features of the mattress and/or external to the mattress.

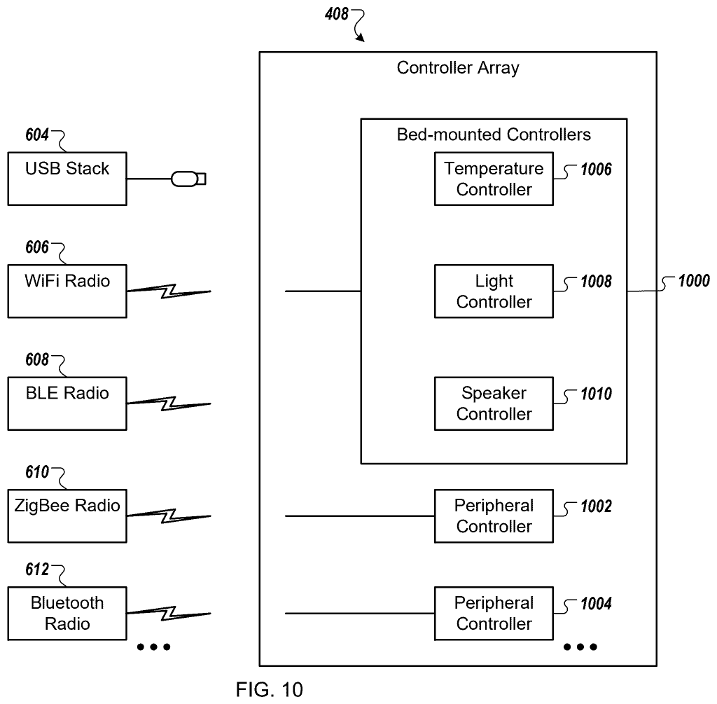

FIG. 10 is a block diagram of an example of the controller array 408 that can be used in a data processing system associated with a bed system, including those described above with respect to FIGS. 1-3. In general, the controller array 408 is a conceptual grouping of some or all peripheral controllers that communicate with the motherboard 402 but are not native to the motherboard 402.

The peripheral controllers of the controller array 408 can communicate with the motherboard 402 through one or more of the network interfaces of the motherboard, including but not limited to the USB stack 604, WiFi radio 606, Bluetooth Low Energy (BLE) radio 608, ZigBee radio 610, and Bluetooth radio 612, as is appropriate for the configuration of the particular sensor. For example, a controller that receives a command over a USB cable can communicate through the USB stack 604.

Some of the controllers of the controller array 408 can be bed mounted controllers 1000, such as a temperature controller 1006, a light controller 1008, and a speaker controller 1010. The bed mounting controllers 1000 can be, for example, embedded into the structure of a bed and sold with the bed, or later affixed to the structure of the bed, as described in reference to the peripheral sensors in FIG. 9. Other peripheral controllers 1002 and 1004 can be in communication with the motherboard 402, but optionally not mounted to the bed. In some cases, some or all of the bed mounted controllers 1000 and/or the peripheral controllers 1002 and 1004 can share networking hardware, including a conduit that contains wires for each controller, a multi-wire cable or plug that, when affixed to the motherboard 402, connects all of the associated controllers with the motherboard 402.

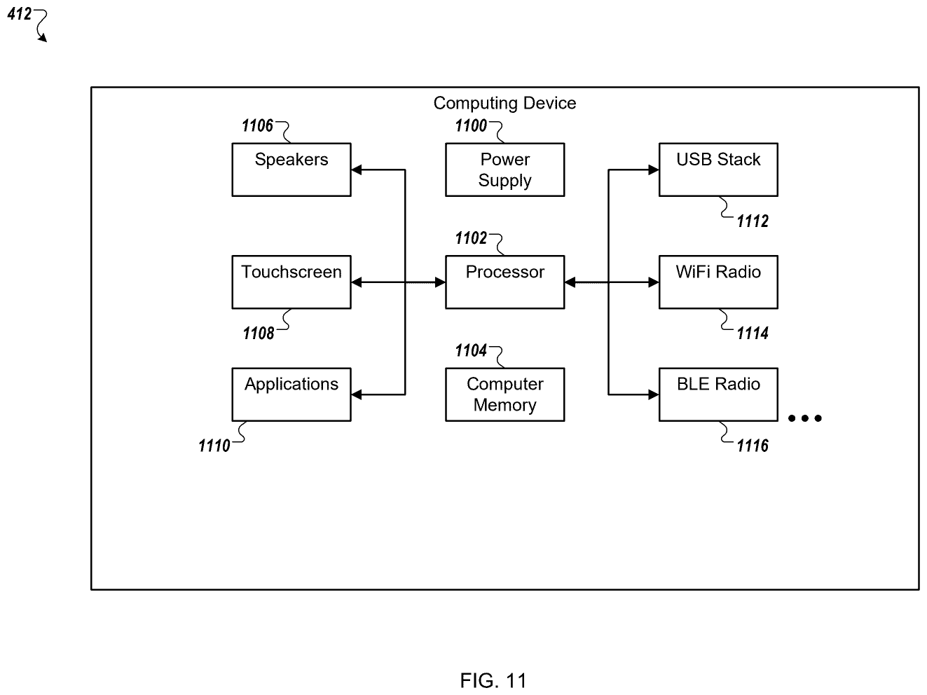

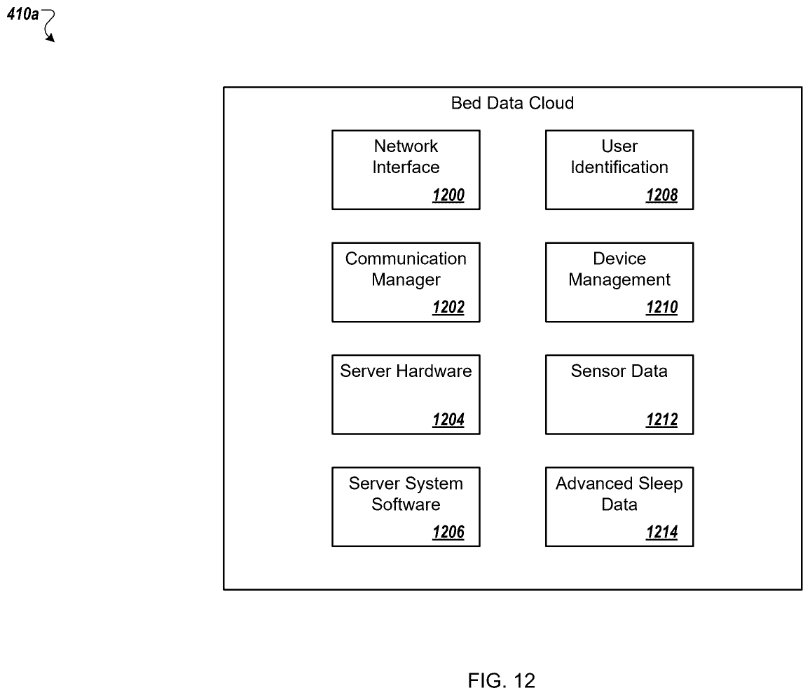

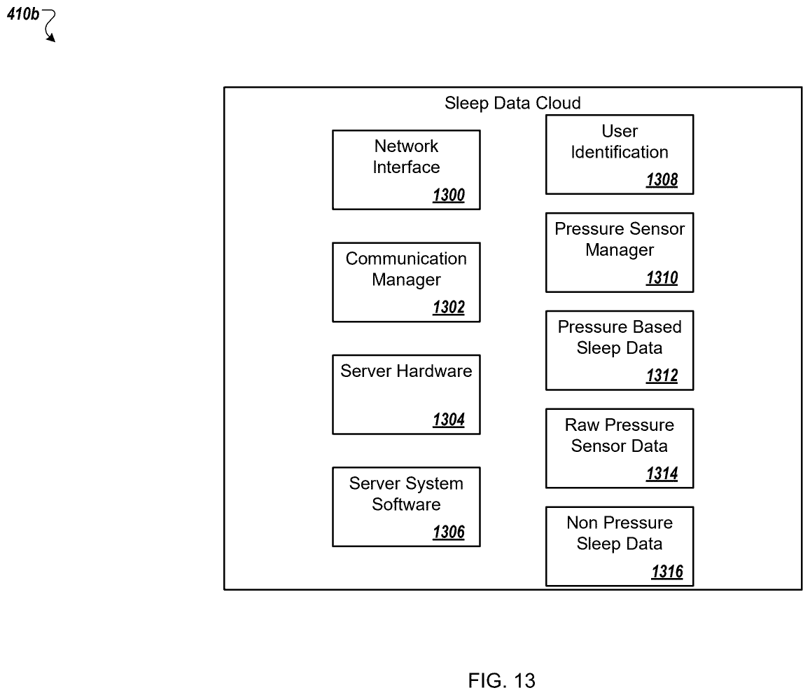

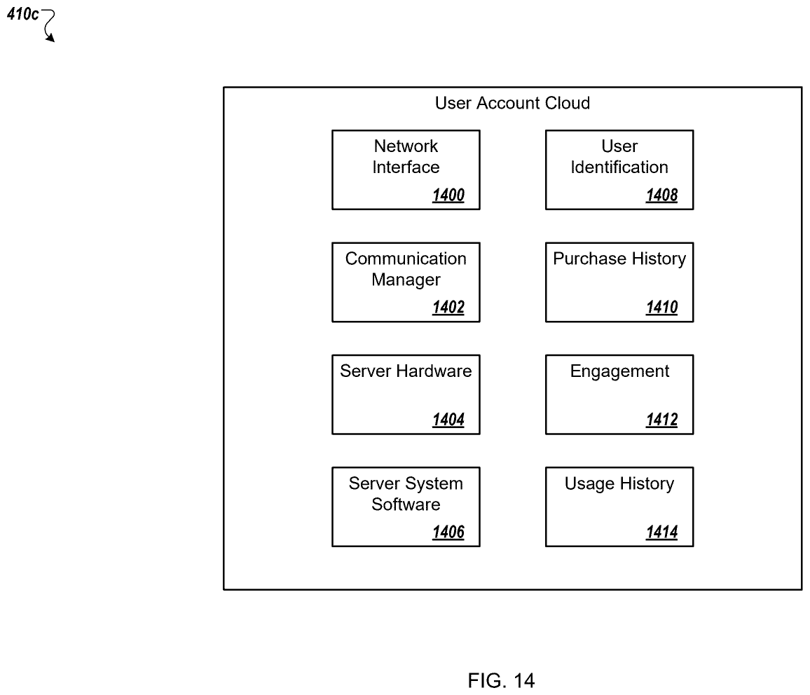

FIG. 11 is a block diagram of an example of the computing device 412 that can be used in a data processing system associated with a bed system, including those described above with respect to FIGS. 1-3. The computing device 412 can include, for example, computing devices used by a user of a bed. Example computing devices 412 include, but are not limited to, mobile computing devices (e.g., mobile phones, tablet computers, laptops, smart phones, wearable devices), desktop computers, home automation devices, and/or central controllers or other hub devices.

The computing device 412 includes a power supply 1100, a processor 1102, and computer readable memory 1104. User input and output can be transmitted by, for example, speakers 1106, a touchscreen 1108, or other not shown components, such as a pointing device or keyboard. The computing device 412 can run one or more applications 1110. These applications can include, for example, applications to allow the user to interact with the system 400. These applications can allow a user to view information about the bed (e.g., sensor readings, sleep metrics), information about themselves (e.g., health conditions that are detected based on signals that are sensed at the bed), and/or configure the behavior of the system 400 (e.g., set a desired firmness to the bed, set desired behavior for peripheral devices). In some cases, the computing device 412 can be used in addition to, or to replace, the remote control 122 described previously.

FIG. 12 is a block diagram of an example bed data cloud service 410a that can be used in a data processing system associated with a bed system, including those described above with respect to FIGS. 1-3. In this example, the bed data cloud service 410a is configured to collect sensor data and sleep data from a particular bed, and to match the sensor and sleep data with one or more users that use the bed when the sensor and sleep data was generated.

The bed data cloud service 410a is shown with a network interface 1200, a communication manager 1202, server hardware 1204, and server system software 1206. In addition, the bed data cloud service 410a is shown with a user identification module 1208, a device management 1210 module, a sensor data module 1210, and an advanced sleep data module 1214.

The network interface 1200 generally includes hardware and low level software used to allow one or more hardware devices to communicate over networks. For example the network interface 1200 can include network cards, routers, modems, and other hardware needed to allow the components of the bed data cloud service 410a to communicate with each other and other destinations over, for example, the Internet 412.

The communication manager 1202 generally comprises hardware and software that operate above the network interface 1200. This includes software to initiate, maintain, and tear down network communications used by the bed data cloud service 410a. This includes, for example, TCP/IP, SSL or TLS, Torrent, and other communication sessions over local or wide area networks. The communication manager 1202 can also provide load balancing and other services to other elements of the bed data cloud service 410a.

The server hardware 1204 generally includes physical processing devices used to instantiate and maintain the bed data cloud service 410a. This hardware includes, but is not limited to, processors (e.g., central processing units, ASICs, graphical processers) and computer readable memory (e.g., random access memory, stable hard disks, tape backup). One or more servers can be configured into clusters, multi-computer, or datacenters that can be geographically separate or connected.

The server system software 1206 generally includes software that runs on the server hardware 1204 to provide operating environments to applications and services. The server system software 1206 can include operating systems running on real servers, virtual machines instantiated on real servers to create many virtual servers, server level operations such as data migration, redundancy, and backup.

The user identification 1208 can include, or reference, data related to users of beds with associated data processing systems. For example, the users can include customers, owners, or other users registered with the bed data cloud service 410a or another service. Each user can have, for example, a unique identifier, user credentials, contact information, billing information, demographic information, or any other technologically appropriate information.

The device manager 1210 can include, or reference, data related to beds or other products associated with data processing systems. For example, the beds can include products sold or registered with a system associated with the bed data cloud service 410a. Each bed can have, for example, a unique identifier, model and/or serial number, sales information, geographic information, delivery information, a listing of associated sensors and control peripherals, etc. Additionally, an index or indexes stored by the bed data cloud service 410a can identify users that are associated with beds. For example, this index can record sales of a bed to a user, users that sleep in a bed, etc.

The sensor data 1212 can record raw or condensed sensor data recorded by beds with associated data processing systems. For example, a bed's data processing system can have a temperature sensor, pressure sensor, motion sensor, audio sensor, and/or light sensor. Readings from one or more of these sensors, either in raw form or in a format generated from the raw data (e.g. sleep metrics) of the sensors, can be communicated by the bed's data processing system to the bed data cloud service 410a for storage in the sensor data 1212. Additionally, an index or indexes stored by the bed data cloud service 410a can identify users and/or beds that are associated with the sensor data 1212.

The bed data cloud service 410a can use any of its available data, such as the sensor data 1212, to generate advanced sleep data 1214. In general, the advanced sleep data 1214 includes sleep metrics and other data generated from sensor readings, such as health information associated with the user of a particular bed. Some of these calculations can be performed in the bed data cloud service 410a instead of locally on the bed's data processing system, for example, because the calculations can be computationally complex or require a large amount of memory space or processor power that may not be available on the bed's data processing system. This can help allow a bed system to operate with a relatively simple controller and still be part of a system that performs relatively complex tasks and computations.

For example, the bed data cloud service 410a can retrieve one or more machine learning models from a remote data store and use those models to determine the advanced sleep data 1214. The bed data cloud service 410a can retrieve different types of models based on a type of the advanced sleep data 1214 that is being generated. As an illustrative example, the bed data cloud service 410a can retrieve one or more models to determine overall sleep quality of the user based on currently detected sensor data 1212 and/or historic sensor data (e.g., which can be stored in and accessed from a data store). The bed data cloud service 410a can retrieve one or more other models to determine whether the user is currently snoring based on the detected sensor data 1212. The bed data cloud service 410a can also retrieve one or more other models that can be used to determine whether the user is experiencing some health condition based on the detected sensor data 1212.

FIG. 13 is a block diagram of an example sleep data cloud service 410b that can be used in a data processing system associated with a bed system, including those described above with respect to FIGS. 1-3. In this example, the sleep data cloud service 410b is configured to record data related to users' sleep experience.

The sleep data cloud service 410b is shown with a network interface 1300, a communication manager 1302, server hardware 1304, and server system software 1306. In addition, the sleep data cloud service 410b is shown with a user identification module 1308, a pressure sensor manager 1310, a pressure based sleep data module 1312, a raw pressure sensor data module 1314, and a non-pressure sleep data module 1316. Sometimes, the sleep data cloud service 410b can include a sensor manager for each of the sensors that are integrated or otherwise in communication with the bed. In some implementations, the sleep data cloud service 410b can include a sensor manager that relates to multiple sensors in beds. For example, a single sensor manager can relate to pressure, temperature, light, movement, and audio sensors in a bed.

Referring to the sleep data cloud service 410b in FIG. 13, the pressure sensor manager 1310 can include, or reference, data related to the configuration and operation of pressure sensors in beds. For example, this data can include an identifier of the types of sensors in a particular bed, their settings and calibration data, etc.

The pressure based sleep data 1312 can use raw pressure sensor data 1314 to calculate sleep metrics specifically tied to pressure sensor data. For example, user presence, movements, weight change, heartrate, and breathing rate can all be determined from raw pressure sensor data 1314. Additionally, an index or indexes stored by the sleep data cloud service 410b can identify users that are associated with pressure sensors, raw pressure sensor data, and/or pressure based sleep data.

The non-pressure sleep data 1316 can use other sources of data to calculate sleep metrics. For example, user-entered preferences, light sensor readings, and sound sensor readings can all be used to track sleep data. Additionally, an index or indexes stored by the sleep data cloud service 410b can identify users that are associated with other sensors and/or non-pressure sleep data 1316.

FIG. 14 is a block diagram of an example user account cloud service 410c that can be used in a data processing system associated with a bed system, including those described above with respect to FIGS. 1-3. In this example, the user account cloud service 410c is configured to record a list of users and to identify other data related to those users.

The user account cloud service 410c is shown with a network interface 1400, a communication manager 1402, server hardware 1404, and server system software 1406. In addition, the user account cloud service 410c is shown with a user identification module 1408, a purchase history module 1410, an engagement module 1412, and an application usage history module 1414.

The user identification module 1408 can include, or reference, data related to users of beds with associated data processing systems. For example, the users can include customers, owners, or other users registered with the user account cloud service 410c or another service. Each user can have, for example, a unique identifier, and user credentials, demographic information, or any other technologically appropriate information. Each user can also have user-inputted preferences pertaining to the user's bed system (e.g., firmness settings, heating/cooling settings, inclined and/or declined positions of different regions of the bed, etc.), ambient environment (e.g., lighting, temperature, etc.), and/or peripheral devices (e.g., turning on or off a television, coffee maker, security system, alarm clock, etc.).

The purchase history module 1410 can include, or reference, data related to purchases by users. For example, the purchase data can include a sale's contact information, billing information, and salesperson information that is associated with the user's purchase of the bed system. Additionally, an index or indexes stored by the user account cloud service 410c can identify users that are associated with a purchase of the bed system.

The engagement 1412 can track user interactions with the manufacturer, vendor, and/or manager of the bed and or cloud services. This engagement data can include communications (e.g., emails, service calls), data from sales (e.g., sales receipts, configuration logs), and social network interactions. The engagement data can also include servicing, maintenance, or replacements of components of the user's bed system.

The usage history module 1414 can contain data about user interactions with one or more applications and/or remote controls of a bed. For example, a monitoring and configuration application can be distributed to run on, for example, computing devices 412. The computing devices 412 can include a mobile phone, laptop, tablet, computer, smartphone, and/or wearable device of the user. The computing devices 412 can also include a central controller or hub device that can be used to control operations of the bed system and one or more peripheral devices. Moreover, the computing devices 412 can include a home automation device. The application that is presented to the user via the computing devices 412 can log and report user interactions for storage in the application usage history module 1414. Additionally, an index or indexes stored by the user account cloud service 410c can identify users that are associated with each log entry. User interactions that are stored in the application usage history module 1414 can optionally be used to determine or otherwise predict user preferences and/or settings for the user's bed and/or peripheral devices that can improve the user's overall sleep quality.

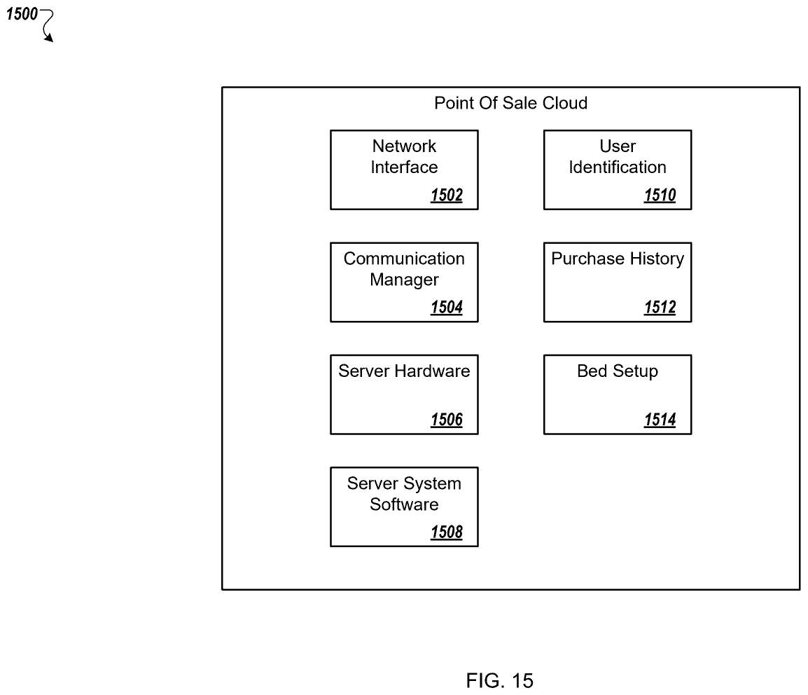

FIG. 15 is a block diagram of an example point of sale cloud service 1500 that can be used in a data processing system associated with a bed system, including those described above with respect to FIGS. 1-3. In this example, the point of sale cloud service 1500 is configured to record data related to users' purchases, specifically purchases of bed systems described herein.

The point of sale cloud service 1500 is shown with a network interface 1502, a communication manager 1504, server hardware 1506, and server system software 1508. In addition, the point of sale cloud service 1500 is shown with a user identification module 1510, a purchase history module 1512, and a bed setup module 1514.

The purchase history module 1512 can include, or reference, data related to purchases made by users identified in the user identification module 1510. The purchase information can include, for example, data of a sale, price, and location of sale, delivery address, and configuration options selected by the users at the time of sale. These configuration options can include selections made by the user about how they wish their newly purchased beds to be setup and can include, for example, expected sleep schedule, a listing of peripheral sensors and controllers that they have or will install, etc.

The bed setup module 1514 can include, or reference, data related to installations of beds that users purchase. The bed setup data can include, for example, a date and address to which a bed is delivered, a person who accepts delivery, configuration that is applied to the bed upon delivery (e.g., firmness settings), name or names of a user or users who will sleep on the bed, which side of the bed each user will use, etc.

Data recorded in the point of sale cloud service 1500 can be referenced by a user's bed system at later dates to control functionality of the bed system and/or to send control signals to peripheral components according to data recorded in the point of sale cloud service 1500. This can allow a salesperson to collect information from the user at the point of sale that later facilitates automation of the bed system. In some examples, some or all aspects of the bed system can be automated with little or no user-entered data required after the point of sale. In other examples, data recorded in the point of sale cloud service 1500 can be used in connection with a variety of additional data gathered from user-entered data.

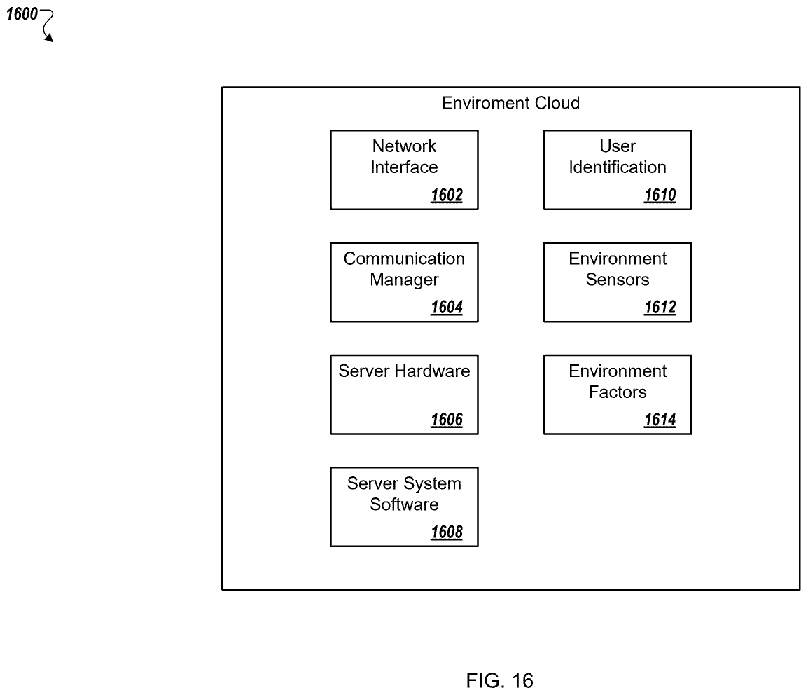

FIG. 16 is a block diagram of an example environment cloud service 1600 that can be used in a data processing system associated with a bed system, including those described above with respect to FIGS. 1-3. In this example, the environment cloud service 1600 is configured to record data related to users' home environment.

The environment cloud service 1600 is shown with a network interface 1602, a communication manager 1604, server hardware 1606, and server system software 1608. In addition, the environment cloud service 1600 is shown with a user identification module 1610, an environmental sensors module 1612, and an environmental factors module 1614.

The environmental sensors module 1612 can include a listing and identification of sensors that users identified in the user identification module 1610 have installed in and/or surrounding their bed. These sensors may include any sensors that can detect environmental variables, including but not limited to light sensors, noise/audio sensors, vibration sensors, thermostats, movement sensors (e.g., motion), etc. Additionally, the environmental sensors module 1612 can store historical readings or reports from those sensors. The environmental sensors module 1612 can then be accessed at a later time and used by one or more of the cloud services described herein to determine sleep quality and/or health information of the users.

The environmental factors module 1614 can include reports generated based on data in the environmental sensors module 1612. For example, the environmental factors module 1614 can generate and retain a report indicating frequency and duration of instances of increased lighting when the user is asleep based on light sensor data that is stored in the environment sensors module 1612.

In the examples discussed here, each cloud service 410 is shown with some of the same components. In various configurations, these same components can be partially or wholly shared between services, or they can be separate. In some configurations, each service can have separate copies of some or all of the components that are the same or different in some ways. Additionally, these components are only provided as illustrative examples. In other examples, each cloud service can have different number, types, and styles of components that are technically possible.

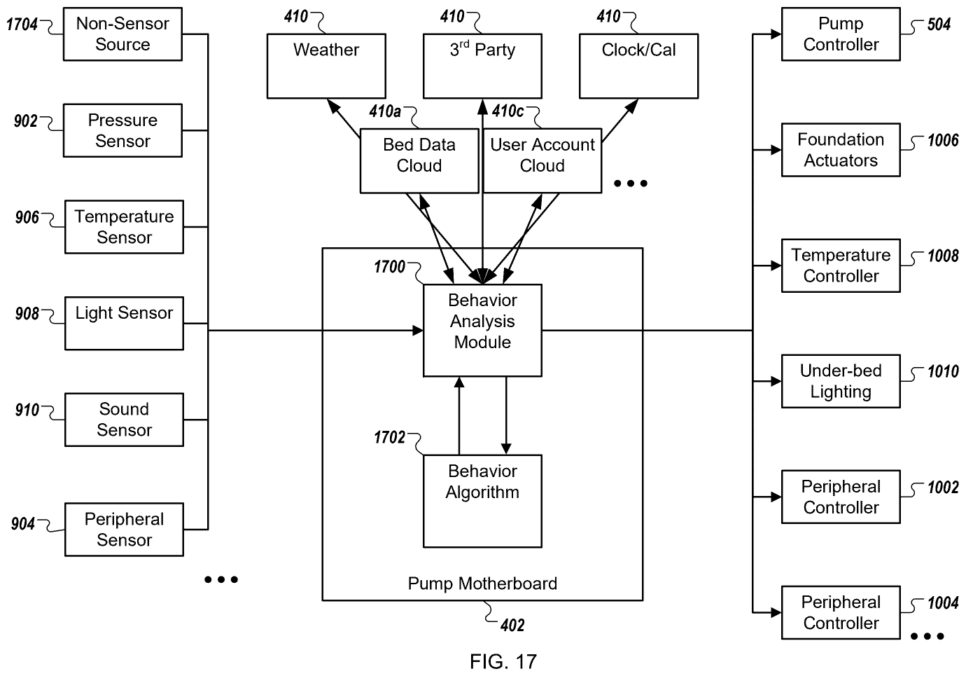

FIG. 17 is a block diagram of an example of using a data processing system associated with a bed (e.g., a bed of the bed systems described herein, such as in FIGS. 1-3) to automate peripherals around the bed. Shown here is a behavior analysis module 1700 that runs on the pump motherboard 402. For example, the behavior analysis module 1700 can be one or more software components stored on the computer memory 512 and executed by the processor 502.

In general, the behavior analysis module 1700 can collect data from a wide variety of sources (e.g., sensors 902, 904, 906, 908, and/or 910, non-sensor local sources 1704, cloud data services 410a and/or 410c) and use a behavioral algorithm 1702 (e.g., one or more machine learning models) to generate one or more actions to be taken (e.g., commands to send to peripheral controllers, data to send to cloud services, such as the bed data cloud 410a and/or the user account cloud 410c). This can be useful, for example, in tracking user behavior and automating devices in communication with the user's bed.

The behavior analysis module 1700 can collect data from any technologically appropriate source, for example, to gather data about features of a bed, the bed's environment, and/or the bed's users. Some such sources include any of the sensors of the sensor array 406 that is previously described (e.g., including but not limited to sensors such as 902, 904, 906, 908, and/or 910). For example, this data can provide the behavior analysis module 1700 with information about a current state of the environment around the bed. For example, the behavior analysis module 1700 can access readings from the pressure sensor 902 to determine the pressure of an air chamber in the bed. From this reading, and potentially other data, user presence in the bed can be determined. In another example, the behavior analysis module 1700 can access the light sensor 908 to detect the amount of light in the bed's environment. The behavior analysis module 1700 can also access the temperature sensor 906 to detect a temperature in the bed's environment and/or one or more microclimates in the bed. Using this data, the behavior analysis module 1700 can determine whether temperature adjustments should be made to the bed's environment and/or components of the bed in order to improve the user's sleep quality and overall comfortability.

Similarly, the behavior analysis module 1700 can access data from cloud services and use such data to make more accurate determinations of user sleep quality, health information, and/or control of the user's bed and/or peripheral devices. For example, the behavior analysis module 1700 can access the bed cloud service 410a to access historical sensor data 1212 and/or advanced sleep data 1214. Other cloud services 410, including those previously described can be accessed by the behavior analysis module 1700. For example, the behavior analysis module 1700 can access a weather reporting service, a 3^{rd} party data provider (e.g., traffic and news data, emergency broadcast data, user travel data), and/or a clock and calendar service. Using data that is retrieved from the cloud services 410, the behavior analysis module 1700 can more accurately determine user sleep quality, health information, and/or control of the user's bed and/or peripheral devices.

Similarly, the behavior analysis module 1700 can access data from non-sensor sources 1704. For example, the behavior analysis module 1700 can access a local clock and calendar service (e.g., a component of the motherboard 402 or of the processor 502). The behavior analysis module 1700 can use the local clock and/or calendar information to determine, for example, times of day that the user is in the bed, asleep, waking up, and/or going to bed.

The behavior analysis module 1700 can aggregate and prepare this data for use with one or more behavioral algorithms 1702. As mentioned, the behavioral algorithm 1702 can include machine learning models. The behavioral algorithms 1702 can be used to learn a user's behavior and/or to perform some action based on the state of the accessed data and/or the predicted user behavior. For example, the behavior algorithm 1702 can use available data (e.g., pressure sensor, non-sensor data, clock and calendar data) to create a model of when a user goes to bed every night. Later, the same or a different behavioral algorithm 1702 can be used to determine if an increase in air chamber pressure is likely to indicate a user going to bed and, if so, send some data to a third-party cloud service 410 and/or engage a peripheral controller 1002 or 1004, foundation actuators 1006, a temperature controller 1008, and/or an under-bed lighting controller 1010.

In the example shown, the behavioral analysis module 1700 and the behavioral algorithm 1702 are shown as components of the pump motherboard 402. However, other configurations are possible. For example, the same or a similar behavioral analysis module 1700 and/or behavioral algorithm 1702 can be run in one or more cloud services, and resulting output can be sent to the pump motherboard 402, a controller in the controller array 408, or to any other technologically appropriate recipient described throughout this document.

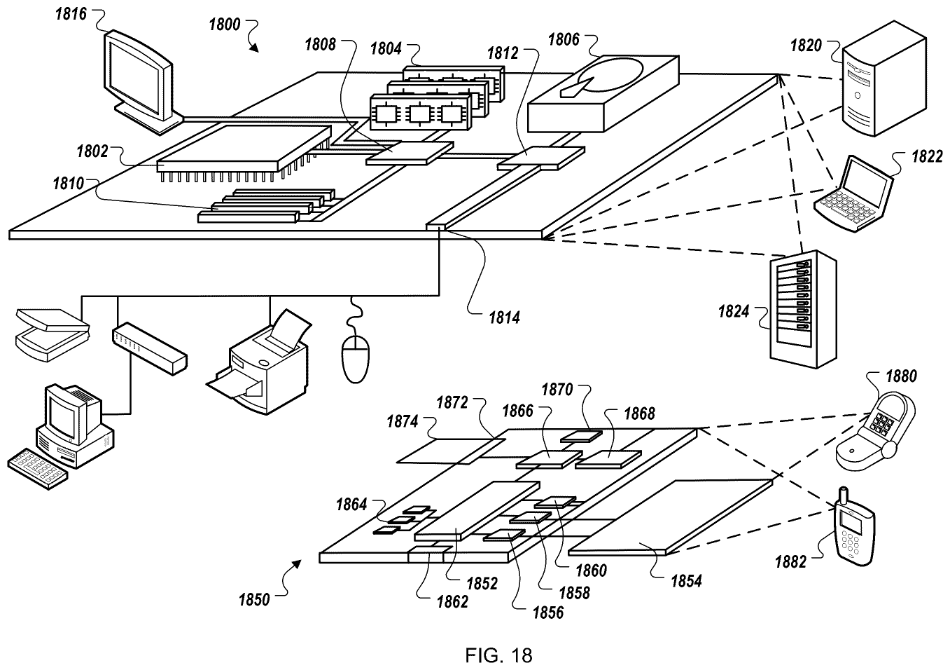

FIG. 18 shows an example of a computing device 1800 and an example of a mobile computing device that can be used to implement the techniques described here. The computing device 1800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1800 includes a processor 1802, a memory 1804, a storage device 1806, a high-speed interface 1808 connecting to the memory 1804 and multiple high-speed expansion ports 1810, and a low-speed interface 1812 connecting to a low-speed expansion port 1814 and the storage device 1806. Each of the processor 1802, the memory 1804, the storage device 1806, the high-speed interface 1808, the high-speed expansion ports 1810, and the low-speed interface 1812, are interconnected using various busses, and can be mounted on a common motherboard or in other manners as appropriate. The processor 1802 can process instructions for execution within the computing device 1800, including instructions stored in the memory 1804 or on the storage device 1806 to display graphical information for a GUI on an external input/output device, such as a display 1816 coupled to the high-speed interface 1808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1804 stores information within the computing device 1800. In some implementations, the memory 1804 is a volatile memory unit or units. In some implementations, the memory 1804 is a non-volatile memory unit or units. The memory 1804 can also be another form of computer-readable medium, such as a magnetic or optical disk.

The storage device 1806 is capable of providing mass storage for the computing device 1800. In some implementations, the storage device 1806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1804, the storage device 1806, or memory on the processor 1802.

The high-speed interface 1808 manages bandwidth-intensive operations for the computing device 1800, while the low-speed interface 1812 manages lower bandwidth-intensive operations. Such allocation of functions is exemplary only. In some implementations, the high-speed interface 1808 is coupled to the memory 1804, the display 1816 (e.g., through a graphics processor or accelerator), and to the high-speed expansion ports 1810, which can accept various expansion cards (not shown). In the implementation, the low-speed interface 1812 is coupled to the storage device 1806 and the low-speed expansion port 1814. The low-speed expansion port 1814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1800 can be implemented in a number of different forms, as shown in the figure. For example, it can be implemented as a standard server 1820, or multiple times in a group of such servers. In addition, it can be implemented in a personal computer such as a laptop computer 1822. It can also be implemented as part of a rack server system 1824. Alternatively, components from the computing device 1800 can be combined with other components in a mobile device (not shown), such as a mobile computing device 1850. Each of such devices can contain one or more of the computing device 1800 and the mobile computing device 1850, and an entire system can be made up of multiple computing devices communicating with each other.

The mobile computing device 1850 includes a processor 1852, a memory 1864, an input/output device such as a display 1854, a communication interface 1866, and a transceiver 1868, among other components. The mobile computing device 1850 can also be provided with a storage device, such as a micro-drive or other device, to provide additional storage. Each of the processor 1852, the memory 1864, the display 1854, the communication interface 1866, and the transceiver 1868, are interconnected using various buses, and several of the components can be mounted on a common motherboard or in other manners as appropriate.

The processor 1852 can execute instructions within the mobile computing device 1850, including instructions stored in the memory 1864. The processor 1852 can be implemented as a chipset of chips that include separate and multiple analog and digital processors. The processor 1852 can provide, for example, for coordination of the other components of the mobile computing device 1850, such as control of user interfaces, applications run by the mobile computing device 1850, and wireless communication by the mobile computing device 1850.

The processor 1852 can communicate with a user through a control interface 1858 and a display interface 1856 coupled to the display 1854. The display 1854 can be, for example, a TFT (Thin-Film-Transistor Liquid Crystal Display) display or an OLED (Organic Light Emitting Diode) display, or other appropriate display technology. The display interface 1856 can comprise appropriate circuitry for driving the display 1854 to present graphical and other information to a user. The control interface 1858 can receive commands from a user and convert them for submission to the processor 1852. In addition, an external interface 1862 can provide communication with the processor 1852, so as to enable near area communication of the mobile computing device 1850 with other devices. The external interface 1862 can provide, for example, for wired communication in some implementations, or for wireless communication in other implementations, and multiple interfaces can also be used.

The memory 1864 stores information within the mobile computing device 1850. The memory 1864 can be implemented as one or more of a computer-readable medium or media, a volatile memory unit or units, or a non-volatile memory unit or units. An expansion memory 1874 can also be provided and connected to the mobile computing device 1850 through an expansion interface 1872, which can include, for example, a SIMM (Single In Line Memory Module) card interface. The expansion memory 1874 can provide extra storage space for the mobile computing device 1850, or can also store applications or other information for the mobile computing device 1850. Specifically, the expansion memory 1874 can include instructions to carry out or supplement the processes described above, and can include secure information also. Thus, for example, the expansion memory 1874 can be provide as a security module for the mobile computing device 1850, and can be programmed with instructions that permit secure use of the mobile computing device 1850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1864, the expansion memory 1874, or memory on the processor 1852. In some implementations, the computer program product can be received in a propagated signal, for example, over the transceiver 1868 or the external interface 1862.

The mobile computing device 1850 can communicate wirelessly through the communication interface 1866, which can include digital signal processing circuitry where necessary. The communication interface 1866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1868 using a radio-frequency. In addition, short-range communication can occur, such as using a Bluetooth, WiFi, or other such transceiver (not shown). In addition, a GPS (Global Positioning System) receiver module 1870 can provide additional navigation- and location-related wireless data to the mobile computing device 1850, which can be used as appropriate by applications running on the mobile computing device 1850.

The mobile computing device 1850 can also communicate audibly using an audio codec 1860, which can receive spoken information from a user and convert it to usable digital information. The audio codec 1860 can likewise generate audible sound for a user, such as through a speaker, e.g., in a handset of the mobile computing device 1850. Such sound can include sound from voice telephone calls, can include recorded sound (e.g., voice messages, music files, etc.) and can also include sound generated by applications operating on the mobile computing device 1850.

The mobile computing device 1850 can be implemented in a number of different forms, as shown in the figure. For example, it can be implemented as a cellular telephone 1880. It can also be implemented as part of a smart-phone 1882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

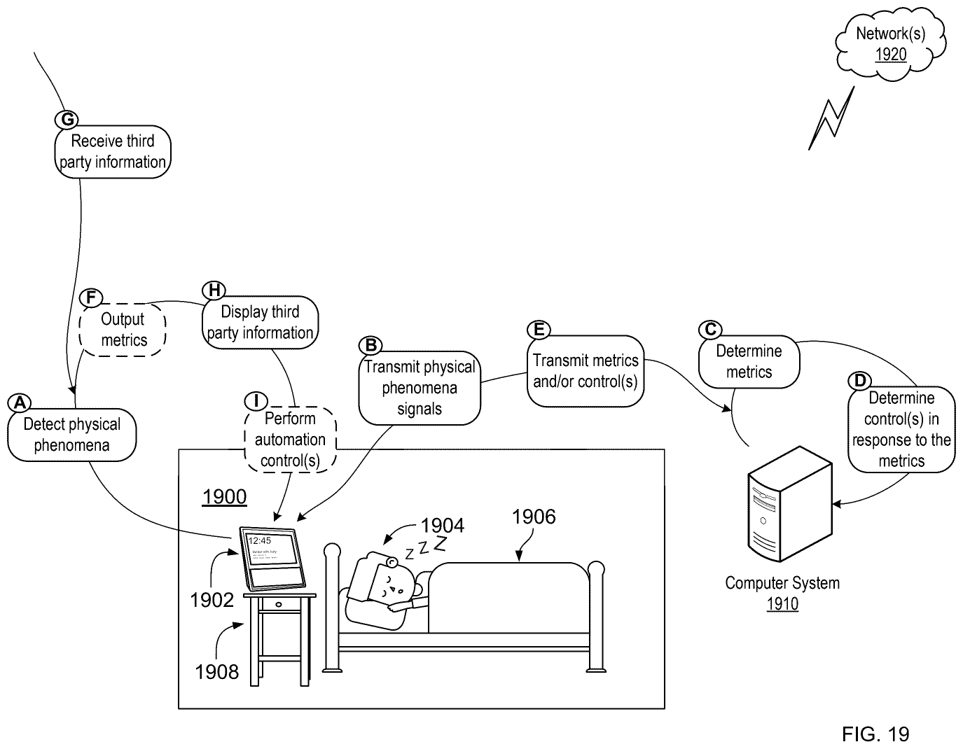

FIG. 19 is an overview conceptual diagram of an environment 1900 having a hub device 1902 that performs the techniques described herein. The environment 1900 can be a bedroom. The environment 1900 can also be any other suitable environment where user 1904 may sleep or nap. In FIG. 19, the user 1904 is sleeping on a bed 1906. The hub device 1904 is located on a nightstand 1908 near the bed 1906. The hub device 1902 can communicate (e.g., wired and/or wireless via network(s) 1920) with a computer system 1910 and/or one or more components in the environment 1900, such as home automation devices depicted and described in FIG. 3. The computer system 1910 can be configured to perform one or more of the processes, techniques, and operations described herein. The computer system 1910 can be remote from the hub device 1902 and/or the environment 1900. The computer system 1910 can also be part of the hub device 1902, in some implementations. Moreover, sometimes the hub device 1902 can perform some of the techniques described herein while the computer system 1910 performs others of the techniques described herein.

The hub device 1902 can be a smart nightstand device that acts as a main console and interface for the user 1904 to interact with a health hub application and other software. The hub device 1902 can include a touchscreen display that can run multiple applications from third parties while also providing the user with environmental, sleep, and health metrics that are determined by the hub device 1902 and/or the computer system 1910. The hub device 1902 may also include a marketplace for partner applications and device integrations, thereby creating a network effect from one singular device. The hub device 1902 can be integrated with electronic medical records (EMRs) to be able to share health-related data with medical and other healthcare providers. As described further, the hub device 1902 can generate a health score based on sensed physical phenomena in the environment 1900 and data associated with the user 1904's sleep and overall health.

The bed 1906 can include smart bed features. For example, the bed 1906 can include an articulable foundation as described above. The bed 1906 can also include an air mattress as previously described.

Although not depicted, home automation devices may also be located in the environment 1900 and/or in communication with one or more of the hub device 1902 and components of the bed 1906. Refer to FIG. 3 for further discussion.

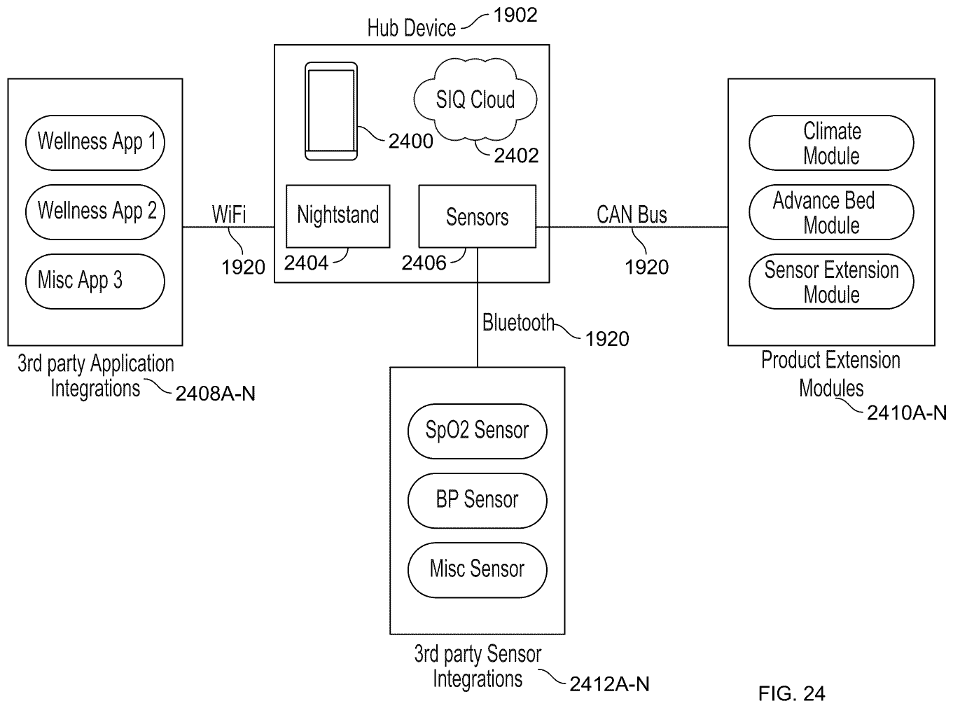

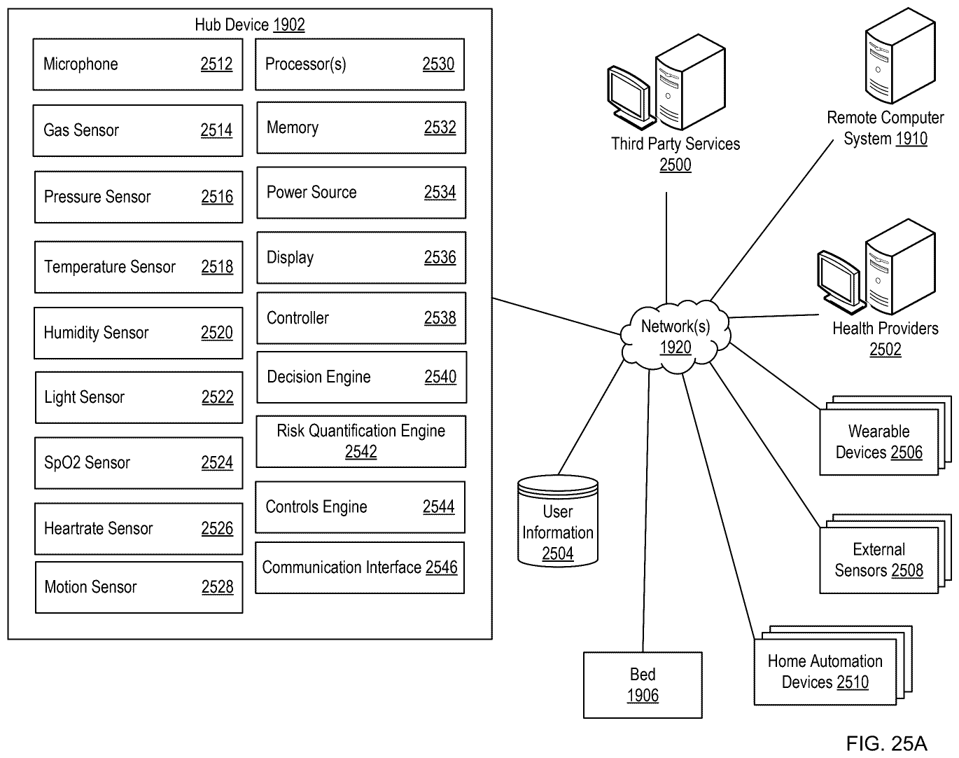

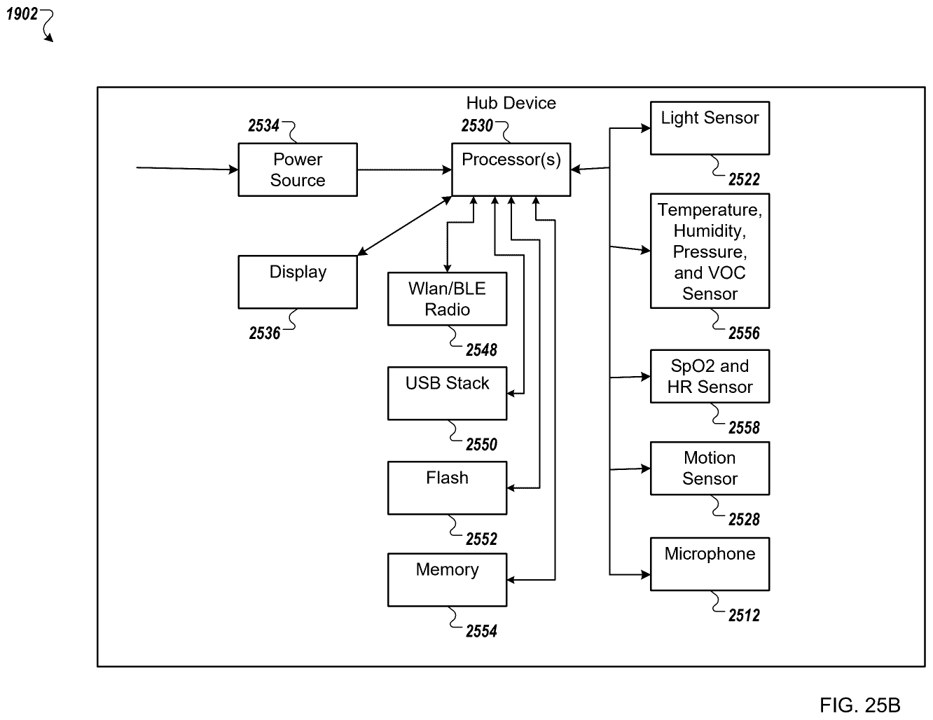

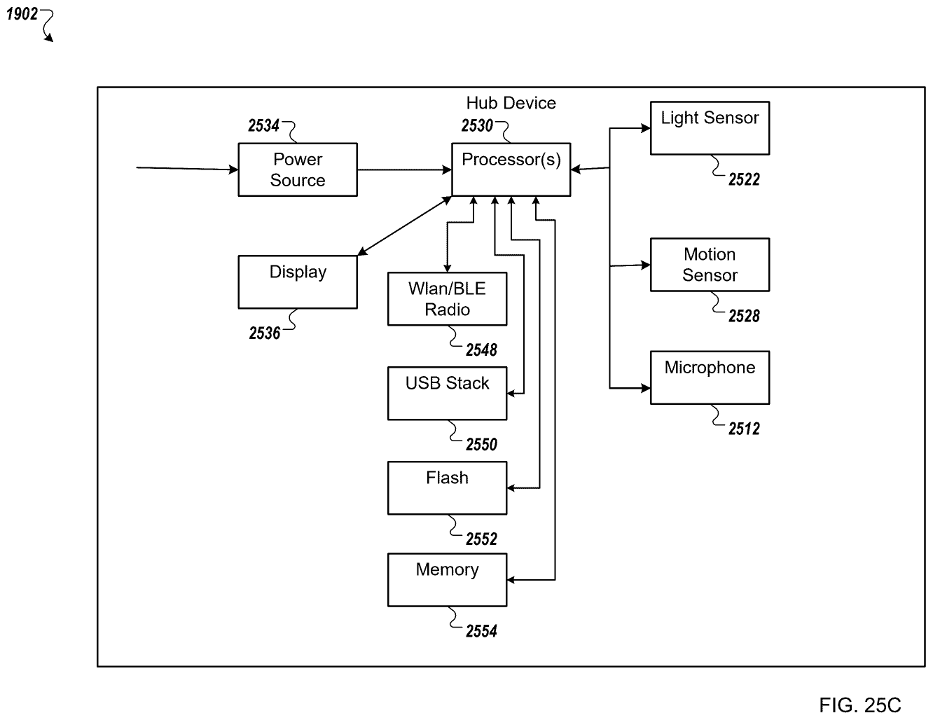

Still referring to FIG. 19, the hub device 1902 can detect physical phenomena in step A. As described further below (e.g., refer to FIGS. 24-25), the hub device 1902 can include a plurality of sensors configured to detect different types of physical phenomena. The hub device 1902 can also be in communication with one or more external sensors positioned throughout the environment 1900, and can receive detected physical phenomena from the external sensors in step A.

The hub device 1902 can transmit the physical phenomena signals to the computer system 1910 in step B. Although not depicted in FIG. 19, in some implementations, the hub device 1902 may not transfer the signals to the computer system 1910. Instead, the hub device 1902 can locally perform steps C-D, which can be advantageous to avoid clogging network bandwidth and to more efficiently utilize computing resources.

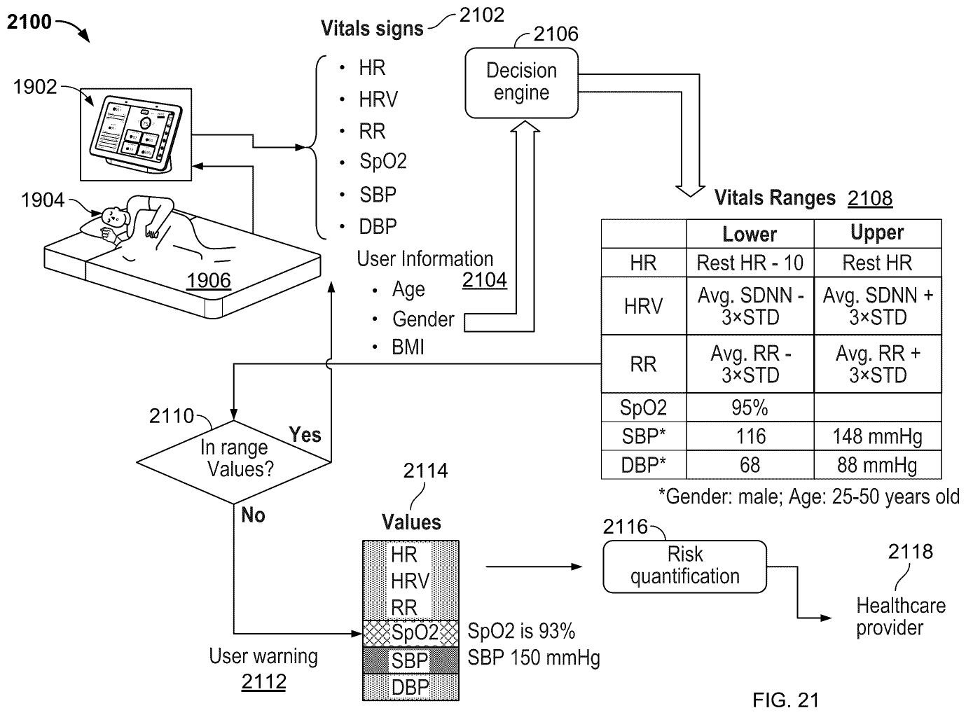

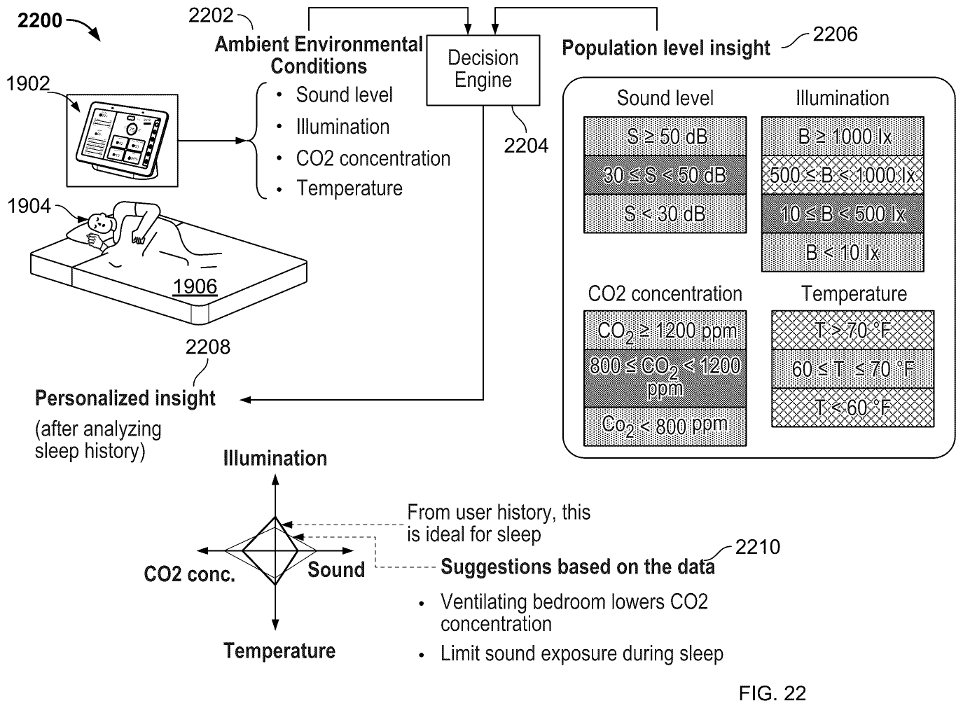

Referring to FIG. 19, the computer system 1910 can determine metrics in step C. Determining metrics can include analyzing the physical phenomena signals to identify environmental, sleep, and health metrics. The computer system 1910 can also determine what conditions in the environment are affecting the user 1904's sleep quality and overall health. Refer to FIGS. 21-22 for additional discussion on determining metrics in step C.

The computer system 1910 can also determine one or more controls in response to the metrics (step D). For example, the computer system 1910 can identify changes that can be made to the environment 1900 in order to improve the user's quality of sleep. Example changes can include adjusting lighting, sound, temperature, and/or humidity in the environment 1900. The computer system 1910 can also determine control signals, instructions, and/or operations that can be executed by the hub device 1902, one or more other controllers, and/or one or more home automation devices to adjust conditions in the environment 1900. As an example, the hub device 1902 can generate instructions that, when transmitted to a home automation device, causes the home automation device to automatically lower blinds over windows in the environment 1900 before the user 1904 goes to sleep. Accordingly, the computer system 1910 can transmit the metrics and/or controls to the hub device in step E. As mentioned above with regards to step B, if the hub device 1902 performs steps C-D, then step E may not be performed. Refer to FIGS. 21-22 for additional discussion on determining controls in step D.

In step F, the hub device 1902 can optionally output the metrics. For example, determined sleep quality information can be presented in a GUI on a display screen of the hub device 1902. As another example, the determined sleep quality information can be locally stored at the hub device 1902 and accessed by the user 1904 via one or more selectable options presented on the display of the hub device 1902. The determined sleep quality information and other metrics can also be stored in a data store (e.g., cloud-base system) and then retrieved by the hub device 1902 when provided with user input requesting to access and view such information.

The hub device 1902 may receive third party information in step G. Step G can be performed at any time, although it is depicted in FIG. 19 as occurring after outputting the metrics in step F. For example, the user 1904 can download third party applications and services to their hub device 1902. Such applications and services can be displayed and accessed at the hub device 1902 by the user 1904. The hub device 1902 can also receive information from medical or other healthcare providers in step G.

In step H, the hub device 1902 can display the third party information. Sometimes, the hub device 1902 may display the information when it is requested by the user 1904 (e.g., the user selects an option on the display—or provides audio input to the hub device 1902—to view a weather application that was downloaded from a marketplace and/or accessible via an Internet connection).

Optionally, the hub device 1902 can perform one or more of the automation controls in step I. Step I can be performed at any time between steps F-H. Sometimes, the hub device 1902 can perform some automation controls while other devices can be instructed, by the computer system 1910, to perform other automation controls. Other times, the hub device 1902 can perform all of the automation controls. Such controls can include, but are not limited to, adjusting settings of the bed 1906 to improve sleep quality (e.g., raising or lowering portions of the foundation, adjusting pressure in one or more air chambers of the mattress, activating heating or cooling elements of the bed 1906, turning on underlighting, etc.), adjusting conditions in the environment to improve sleep quality (e.g., turning lights on or off, turning an HVAC system on or off, opening or closing blinds, adjusting noise levels, adjusting humidity levels, etc.), and/or controlling other devices or components in the user 1904's home (e.g., locking or unlocking doors, starting a coffee machine, turning off a TV or other electronics, activating a security system, etc.).

Steps A-I can be repeated throughout a duration of the user 1904's sleep cycle. Sometimes, one or more of the steps A-I can be performed only upon detecting presence of the user 1904 in the environment 1900 (e.g., based on pressure readings from pressure sensors on the bed 1906). Other times, one or more of the steps A-I can be performed continuously. For example, adjustments can be made to the environment 1900 (e.g., such as adjusting temperature and lighting) before the user 1904 is expected to go to sleep such that the environment 1900 is already prepared for the user 1904 when the user 1904 goes to bed. Preparing the environment 1900 for the user 1904 can be advantageous to improve the user 1904's ability to fall asleep, remain asleep, and experience improved sleep quality.

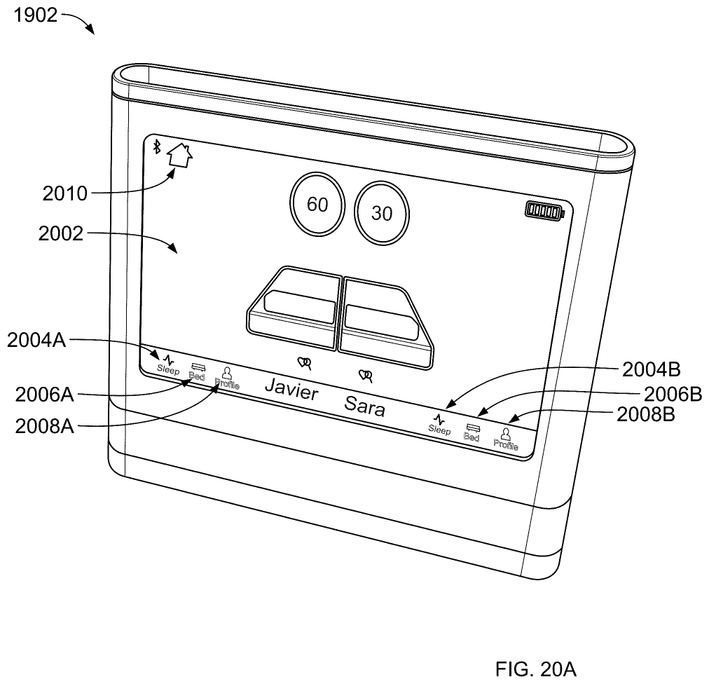

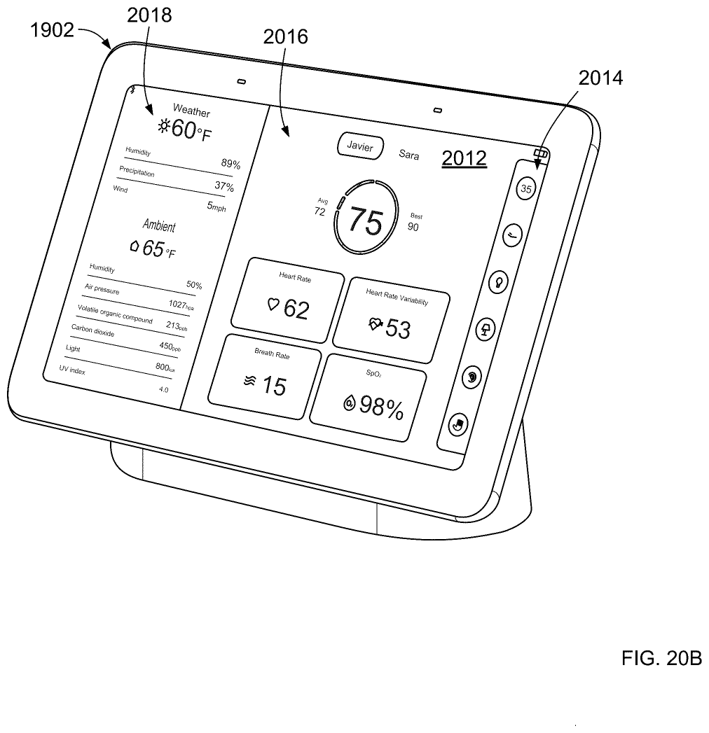

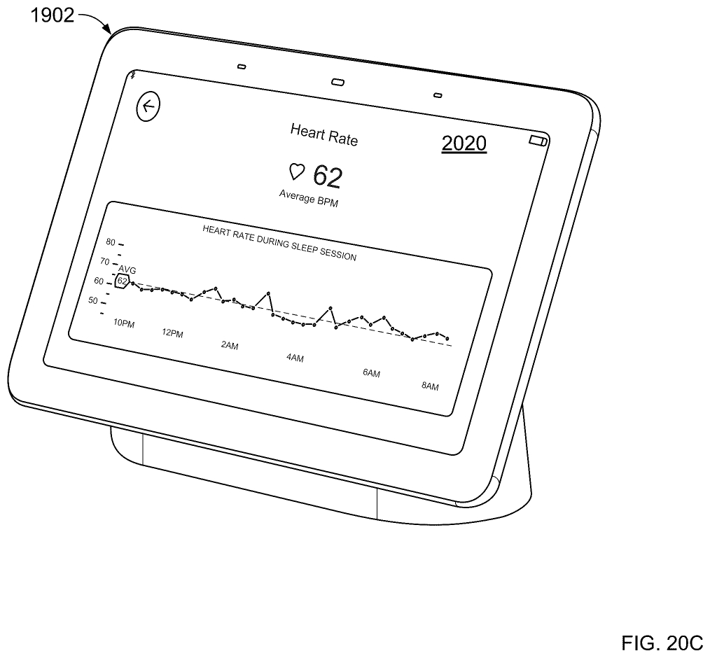

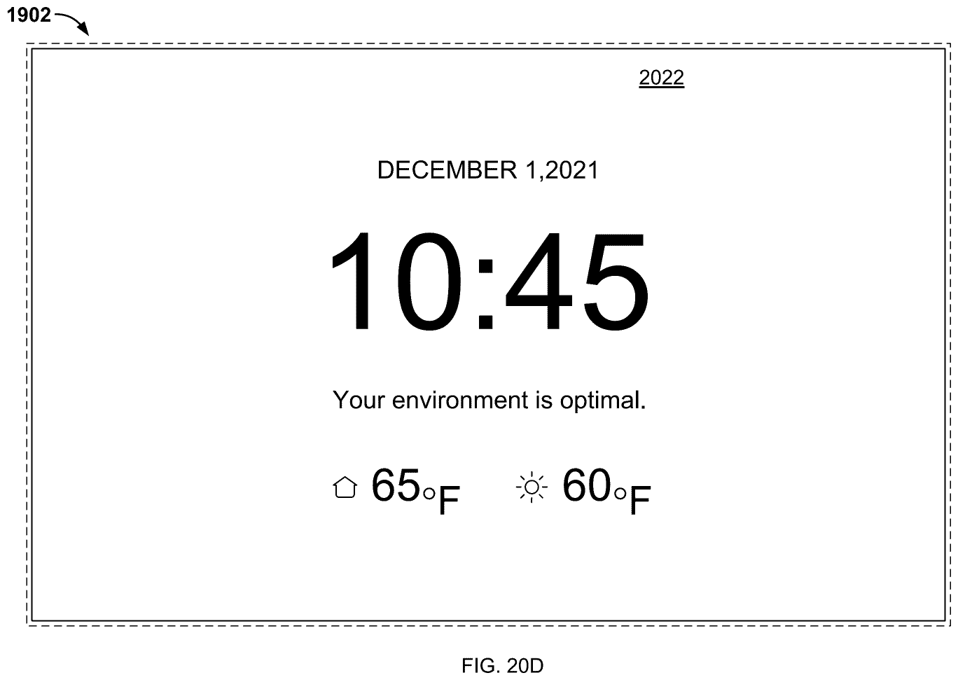

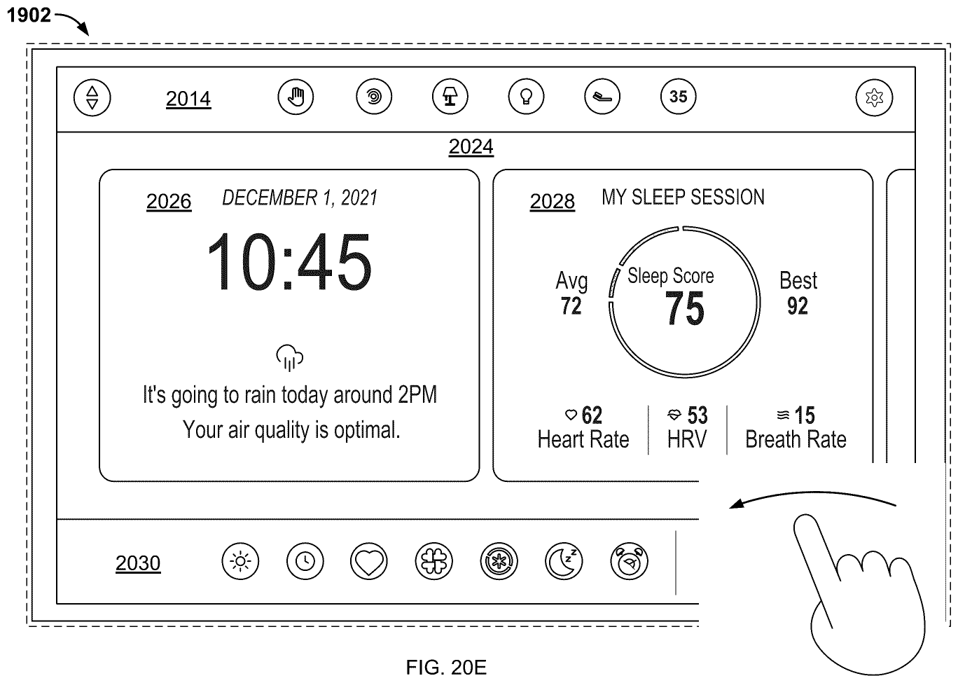

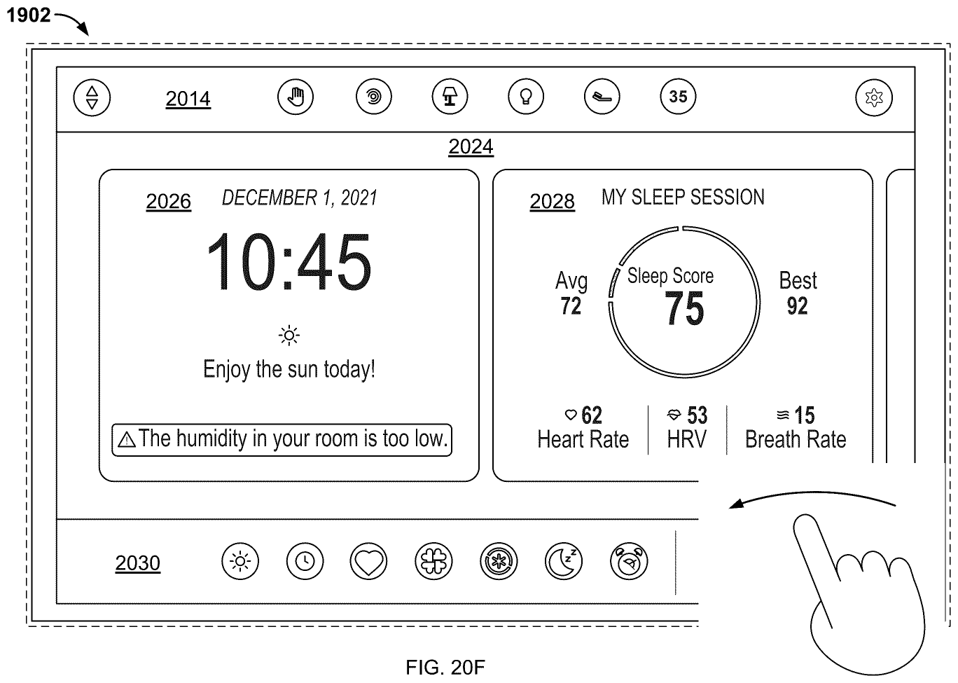

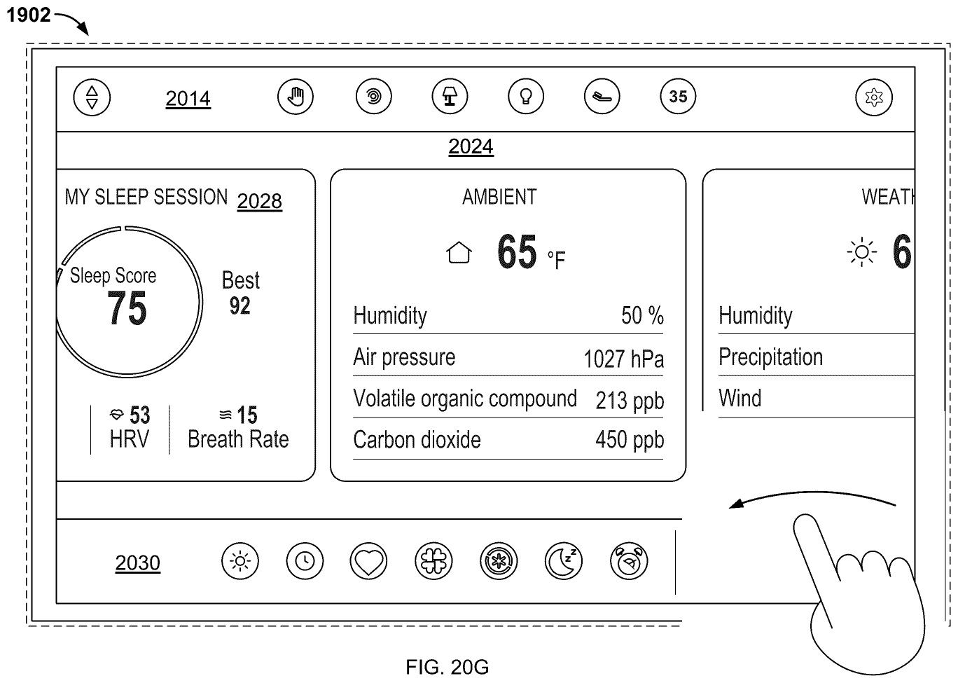

FIGS. 20A-G depict example graphical user interfaces (GUIs) presented at the hub device 1902.

As shown in FIG. 20A, GUI 2002 can provide numerous functionality and information to a user. Moreover, additional GUIs can be presented at the hub device 1902, for example, based on user input requesting to view different information at the hub device 1902. Refer to FIGS. 20B-G for additional GUIs.

The GUI 2002 includes a selectable home option 2010. The user can select this option (e.g., by touching the display screen of the hub device 1902 or providing audio input/speaking to the hub device 1902) in order to return to a home screen or other GUI. For example, the user can be presented a home screen that includes selectable options for viewing information regarding home automation, smart home integration, third party services and applications, environmental metrics, health metrics, and/or sleep metrics. The home screen can present one or more other information.

The GUI 2002 depicts bed control features for two sleepers of the bed: Javier and Sara. Each sleeper has different and independent sleep settings as well as sleep scores or other sleep metrics. For example, Javier has a pressure setting of 60 and Sara has a pressure setting of 30. For each of the sleepers, the GUI 2002 presents multiple selectable options. For example, for the left side of the bed (Javier's side of the bed), the user can select a sleep option 2004A, a bed option 2006A, or a profile option 2008A. Similarly, for the right side of the bed (Sara's side of the bed), the user can select a sleep option 2004B, a bed option 2006B or a profile option 2008B. Accordingly, both sleepers can use the singular hub device 1902 in order to view information and control their respective sides of the bed.

The sleep options 2004A and 2004B allow the respective users to view their sleep metrics and other sleep-related information. Sometimes, a portion of the GUI 2002 can be updated to reflect or display the sleep-related information. For example, if the sleep option 2004A is selected, the left side of the GUI 2002 can be updated to display sleep-related information for Javier, who sleeps on the left side of the bed. In some implementations, the entire GUI 2002 can be updated to display the sleep-related information for the selected side or sides of the bed. Thus, an image of the bed can be replaced with the sleep-related information.

The bed options 2006A and 2006B allow the respective users to view and change settings of their sides of the bed. For example, each of the users can increase or decrease pressure in one or more air chambers on their respective sides of the bed. The users can also raise or lower portions of their sides of the bed. The users can also adjust heating or cooling elements and/or turn massage features on and off on their sides of the bed. When either of the bed options 2006A and 2006B are selected, the GUI 2002 can be updated to display controls for adjusting settings of the respective side of the bed. Another GUI can also be presented at the hub device 1902 to provide functionality for the user(s) to modify settings of the respective side of the bed.

The profile options 2008A and 2008B allow the respective users to view and change their personal information and settings. The profiles can include information such as the user's name, weight, height, and other health metrics. Those health metrics can be inputted by the user and/or provided to the hub device 1902 by medical or healthcare providers. The health metrics can also be determined by the hub device 1902 based on one or more sensed physical phenomena. The profiles can also include information such as the user's sleep score (e.g. as determined by the hub device 1902, the bed, or another computer system), health quality score (e.g. as determined by the hub device 1902, the bed, or another computer system), bed setting preferences like pressure, heating, cooling, massage, incline or decline of portions of the bed (e.g., as inputted by the user and/or determined by the hub device 1902, the bed, or another computer system), environmental setting preferences like temperature, lighting, locking doors (e.g., as inputted by the user and/or determined by the hub device 1902, the bed, or another computer system), bedtime (e.g., as inputted by the user and/or determined by the hub device 1902, the bed, or another computer system), and wakeup time (e.g., as inputted by the user and/or determined by the hub device 1902, the bed, or another computer system). When either of the profile options 2008A and 2008B are selected, the GUI 2002 can be updated to display controls for modifying user profile information. Another GUI can also be presented at the hub device 1902 to provide functionality for the user(s) to modify their profile(s).

The GUI 2002 can include one or more other selectable options.

FIG. 20B depicts another GUI 2012 presented at the hub device 1902. In the GUI 2012, a menu of options 2014 can be displayed with panels 2016 and 2018. The menu 2014 can include one or more selectable options to navigate to different GUIs and/or to view different/additional information. The menu 2014 can also include selectable options to control one or more features and/or devices in a bedroom environment (e.g., turning a light on and off, articulating or otherwise adjusting the bed, etc.).

The panel 2016 can include information about a particular sleeper, similar to what was described in FIG. 20A. For example, the panel 2016 can present an average and best sleep score of the particular user (in FIG. 20B, the particular user is Javier), a heartrate, heartrate variability, breathing rate, and SpO₂. In some implementations, the particular user can select or click on any of the information presented in the panel 2016 to view additional information about the selected metric. For example, selecting the heartrate metric can cause the hub device 1902 to present a GUI such as GUI 2020 in FIG. 20C.

The panel 2018 can include additional information that can be detected and/or determined by the hub device 1902. In the example of FIG. 20B, the panel 2018 presents weather and ambient information. One or more of the weather and ambient information can be detected/determined by the hub device 1902 as described herein. In some implementations, a user can customize what information is presented in the panel 2018 and/or the panel 2016.

FIG. 20C depicts the GUI 2020. The GUI 2020 can be presented when the user selects the heartrate metric in the panel 2016 depicted and described in the GUI 2012 of FIG. 20B. The GUI 2020 can present additional information about the heartrate metric than what is presented in the GUI 2012 in FIG. 20B. For example, one or more graphs can be generated by the hub device 1902 that demonstrate overall heartrate of the user during a sleep session (e.g., a last or most recent sleep session).

FIG. 20D depicts a GUI 2022 presented at the hub device 1902. The GUI 2022 can be a home screen. In other words, the GUI 2022 can be presented at the hub device 1902 until a user toggles to another GUI. As another example, the GUI 2022 can be presented at the hub device 1902 while the user is sleeping or otherwise not interacting with the hub device 1902. Therefore, the GUI 2022 can resemble a simplified interface that may be presented at a home automation device or an alarm clock device.

The user can customize what information is presented in the GUI 2022. Here, the GUI 2022 includes date and time information. The GUI 2022 also includes weather and ambient information (e.g., temperature). The GUI 2022 may also include one or more suggestions for improving the user's sleep environment. In the example of FIG. 20D, the GUI 2022 includes text that says, “Your environment is optimal.” In other examples, the GUI 2022 can include text that prompts the user to change one or more features in the sleep environment, such as turning on an AC of heating system.

FIGS. 20E-G depict another GUI 2024 presented at the hub device 1902. The GUI 2024 can be a main interface for viewing various information and metrics. The GUI 2024 can include the menu 2014 described in reference to FIG. 20B. As shown in FIGS. 20B and 20E, the menu 2014 can be presented in various locations in a GUI. For example, the menu 2014 can be a menu bar positioned at a top of the GUI. The menu 2014 can also be a side bar positioned at a side of the GUI. In some implementations, the menu 2014 can also be a menu bar positioned at a bottom of the GUI.

The GUI 2024 can also include an options bar 2030. The options bar 2030 can include selectable icons that can be used to navigate to one or more other applications provided by and/or presented at the hub device 1902. Example applications can include, but are not limited to, ventilation control, light(s) on/off controls, brightness level control, bed articulation control, and noise masking control.

Moreover, between the menu 2014 and the options bar 2030, the GUI 2024 can present multiple panels. The user can customize what information is presented in the panels and/or how many panels can be displayed in the GUI 2024. The user can swipe across the GUI 2024 to navigate between the panels presented therein.

As shown in FIG. 20E, a panel 2026 can present general information, such as a current date, time, weather, and/or ambient information. A panel 2028 can present sleep session information to the user. The panel 2028 can also present biometrics data that was detected and/or determined by the hub device 1902 during the user's sleep session.

FIG. 20F depicts another example of the GUI 2024. Here, the panel 2026 includes a notification to the user about their ambient environment. The notification indicates that, “The humidity in your room is too low.” In some implementations, the panel 2026 can include one or more suggestions for improving the humidity level in the room. For example, the user can click on the notification to then be presented another GUI having one or more selectable controls for automatically adjusting humidity in the room.

As shown in FIG. 20G, when the user swipes across the GUI 2024, the panels 2026 and 2028 can shift to a left of the GUI 2024. The panel 2026 may no longer be visible in the GUI 2024. The panel 2028 may be partially visible in the GUI 2024. In this example, when the user swipes across the GUI 2024 towards a left side of the hub device 1902, a panel 2034 begins to appear in the GUI 2024. The panel 2034 includes weather information. As described throughout this disclosure, the panel 2034 can present other information that is selected by the user. One or more additional or fewer panels can also be presented in the GUI 2024 as the user swipes left or right across the GUI 2024.

As described further below, the hub device 1902 can have multiple different sensors that detect physical phenomena in a surrounding environment. The detected physical phenomena can be used by the hub device 1902 to determine metrics and information that is presented to the user in the GUI 2002 and/or other GUIs. For example, the hub device 1902 can include (and/or be in communication with) sensors that detect ambient light (e.g., visible, UV, and/or IR), temperature, humidity, volatile organic compounds (VOC), electromagnetic interference, atmospheric pressure, SpO₂, pulse, motion, microphone, and/or radar.

The hub device 1902 can also provide different functionality at the GUI 2002. Example functionality includes, but is not limited to, bed controls, sleep quality, sleep metrics, sleep data, a night light, a smart alarm clock, a speaker that receives input and provides output to the user, a smart picture frame, and/or third party applications and services that can be downloaded to the hub device 1902 from a marketplace or otherwise accessible over an Internet connection.

In some implementations, the hub device 1902 can also provide one or more cloud-based services to the user. For example, the cloud-based services can include a health dashboard (e.g., including and/or based on information received from medical or healthcare providers, remote computing systems, sensors, and/or data stores), weather data, stock quotes, integration with third-party automation services, security integration, lighting integration, and/or HVAC integration.

Moreover, the hub device 1902 can provide one or more health services to the user(s) of the bed. The health services can include extensive health dashboards, integration with EMRs, remote health reporting and communication with medical or healthcare providers, and/or remote monitoring by medical or healthcare providers. As a result, health conditions detected by the hub device 1902 and/or one or more components described herein can be used to provide for health monitoring and early detection of, and response to, health issues or conditions.

FIG. 21 is a conceptual diagram for monitoring vital signs 2102 of the user 1904 and assessing a need to report such vital signs 2102 to a healthcare provider 2118. FIG. 21 depicts a process 2100 for monitoring the vital signs 2102. As shown, the user 1904 is sleeping in the bed 1906. The hub device 1902 having its plurality of sensors is positioned in an environment surrounding the user 1904, near the bed 1906. The sensors of the hub device 1902 can detect physical phenomena. Physical phenomena can be continuously detected throughout a sleep cycle of the user 1904. The physical phenomena can also be detected at predetermined time intervals (e.g., every minute, every 5 minutes, etc.). Sometimes, the physical phenomena can be detected when a rare or unusual event occurs (e.g., a crashing sound, a sudden increase in shortness of breath, etc.). In yet some implementations, the physical phenomena can be detected once user presence is detected in the bed 1906 and/or near the hub device 1902.

Sensors on or integrated in the bed 1906 can also detect physical phenomena and transmit the physical phenomena to the hub device 1902. In the process 2100, the physical phenomena that is detected includes vital signs 2102. As described in reference to FIG. 22, the physical phenomena can also be ambient environmental conditions. The vital signs 2102 include heartrate (HR), heartrate variability (HRV), respiration rate (RR), oxygen saturation (SpO₂), systolic blood pressure (SBP), and diastolic blood pressure (DBP).

Such detected vital signs 2102 can be transmitted to a decision engine 2106 for processing. The decision engine 2106 can be part of the hub device 1902. The decision engine 2106 can also be part of a remote computer system, device, and/or cloud-based service. The decision engine 2106 can also receive user information 2104, which can be used to determine whether the user 1904 is experiencing any health related issues while sleeping in the bed 1906. The user information 2104 can be retrieved from a data store. The user information 2104 can also be received from a medical or healthcare provider or EMRs. Sometimes, the user information 2104 can be inputted by the user 1904 at the hub device 1902 and transmitted from the hub device 1902 to the decision engine 2106. The user information 2104 can be specific to the particular user 1904. Sometimes, the user information 2104 can be generic to a class or category of users that the user 1904 is part of. As shown in FIG. 21, the user information 2104 can include the user 1904's age, gender, and/or BMI.

The decision engine 2106 can use the vital signs 2102 and the user information 2104 in order to determine vitals ranges 2108 for the particular user 1904. The decision engine 2106 can also determine whether the particular user 1904 is currently within healthy ranges for the determined vitals ranges 2108.

The vitals ranges 2108 can be determined for each of the vital signs 2102 that are detected at the hub device 1902. In the example of FIG. 21, the vitals ranges 2108 include high and low ranges for HR, HRV, RR, SpO₂, SBP, and DBP. The high and low ranges for HR, HRV, RR, and SpO₂ can be the same regardless of age and/or gender of the user 1904. The SBP and DBP high and low ranges can be specific to the user 1904's age and/or gender. High and low ranges for one or more of the vital signs 2102 can also be based on the user 1904's age, gender, or other health-related information.

In the example of FIG. 21, the user 1904 is a male between 25 and 50 years old. Thus, the high and low ranges for the SBP and DBP are determined based on this gender and age information. The lower range for HR is the user 1904's resting HR minus 10. The upper range for HR is the user 1904's resting HR. The lower range for HRV is the average standard deviation of normal to normal (SDNN) intervals minus 3 times the standard deviation (STD). The upper range for HRV is the average SDNN plus 3 times the STD. SDNN can be measured in milliseconds (ms) and can be calculated over a 24 hour period of time. The lower range of RR can be the average RR minus 3 times the STD. The upper range of RR can be the average RR plus 3 times the STD. The lower range of SpO₂ can be 95%. SpO₂ may not have an upper range. The lower range of SBP, based on the user 1904's age and gender, is 116 mmHg. The upper range of SBP is 148 mmHg. The lower range of DBP, based on the user 1904's age and gender, is 68 mmHg. The upper range of DBP is 88 mmHg. The ranges described herein may be used for a variety of populations of users. For example, these ranges can apply to users in an age range of 20 to 50 years old. However, ranges may also dynamically change according to demographic factors. For example, a 60 year old male can have a normal average SBP (systolic BP) of 140 mmHg and an average DBP (diastolic BP of 75 mmHg). On the other hand, a 60 year old female can have a normal average SBP of 130 mmHg and an average DBP of 75 mmHg. These different ranges can be stored in a data store and retrieved based on user demographics information to perform the techniques described below.

Sometimes, a remote computing system, the hub device 1902, or a cloud-based computing service can determine the vitals ranges 2108 and store the vitals ranges 2108 in a data store. During run time (e.g., during a sleep cycle of the user 1904), the decision engine 2106 can retrieve the vitals ranges 2108 from the data store and use it to determine whether the user 1904 is currently operating within the vitals ranges 2108.

Once the vitals ranges 2108 are determined and/or retrieved from a data store, the decision engine 2106 can determine whether the detected vital signs 2102 are within the vitals ranges 2108 (2110). If the vital signs 2102 are within the vital ranges 2108, then monitoring of the vitals can continue. If any of the vital signs 2102 are not within the vitals ranges 2108, the decision engine 2106 can generate a user warning 2112.

The user warning 2112 can be based on determining, by the decision engine 2106, which of the vital signs 2102 are not within the vitals ranges 2108. Values 2114 indicate which of the vital signs 2102 are within the vitals ranges 2108 and do not require warning the user 1904 and which of the vital signs 2102 are not within the vitals ranges 2108 and should be reported to the user 1904. HR, HRV, RR, and DBP are depicted in green in the values 2114 table. The green color can indicate that such vital signs 2102 are within the vitals ranges 2108. An orange color can indicate a minor deviation from an expected range. For example, SpO₂ is depicted in the orange color since the SpO₂ of the user 1904 is currently 93%. 93% is less than the lower range for the SpO₂ (95%). Thus, the user 1904 should be warned about the SpO₂. Similarly, the SBP of the user 1904 is currently 150 mmHg, which is greater than the upper range (148 mmHg) of the SBP. Therefore, the user should be warned about the SBP. Some values in the values 2114 table can be represented in a red color, which can indicate a substantial deviation from an expected range for that value.

Next, the decision engine 2106 can perform risk quantification 2116. Risk quantification 2116 can be performed to determine whether the vital signs 2102 are trending so outside of the vitals ranges 2108 that the vital signs 2102 should be reported out to the healthcare provider(s) 2118. The decision engine 2106 can detect any deviation outside reference values for any metrics, such as the vital signs 2102. As another example, the decision engine 2106 can execute one or more machine learning trained models to quantify risk. For example, a model can be trained to calculate: z=w1*HR+w2*HRV+w3*RR+w4*SpO₂+w5*BP and to estimate a risk level based on: Risk=1/(1+exp(−z)), where the coefficients of w1, w2, w3, w4, and w5 are obtained during training of the model. Additionally, if the decision engine 2106 determines that the vital signs 2102 should be reported out to the healthcare provider(s) (2118), the decision engine 2106 can encrypt the vital signs 2102 to preserve user privacy rights.

Risk quantification 2116 can also be performed to determine an overall health index for the user 1904, which can be based on all the values 2114 for the vital signs 2102. The health index can, for example, be based on sleep session data, SpO₂, and blood pressure.

In this example of the process 2100, the decision engine 2106 can determine that the SpO₂ and SBP values 2114 should be reported to the healthcare provider 2118 because they pose a significant risk to the user 1904's overall health and/or sleep. The decision engine 2106 can also determine that the user 1904's overall health index should be reported out to the healthcare provider 2118 since it is based on values 2114 for SpO₂ and SBP that are trending outside of their respective vitals ranges 2108. Reporting the values 2114 can include generating a message that is transmitted to a secure portal of the healthcare provider 2118. In some implementations, the user 1904 can control what information is communicated to the healthcare provider 2118.

FIG. 22 is a conceptual diagram for determining changes to make to an environment based on monitoring ambient environmental conditions 2202. Similar to FIG. 21, FIG. 22 depicts a process 2200 for monitoring the ambient environmental conditions 2202. The processes 2100 and 2200 can be performed separately or together. Similar to the process 2100 in FIG. 21, in the process 2200, sensors of the hub device 1902 can detect ambient environmental conditions 2202. The conditions 2202 can be detected at predetermined time intervals (e.g., every minute, every 5 minutes, etc.). Sometimes, the conditions 2202 can be detected when a rare or unusual event occurs (e.g., a crashing sound, a sudden rise in temperature, etc.). In yet some implementations, the conditions 2202 can be detected once user presence is detected in the bed 1906 and/or near the hub device 1902 (e.g., by motion sensors of the hub device 1902).

The ambient environmental conditions 2202 that are detected by sensors of the hub device 1902 can include sound level, illumination/lighting, CO₂ concentration, and temperature. One or more other ambient environmental conditions can also be detected.

The conditions 2202 can be transmitted to decision engine 2204 once the conditions 2202 are detected. The decision engine 2204 can be same or similar to the decision engine 2104 described in FIG. 21. The decision engine 2204 can also receive population level insight 2206, which can be used with the conditions 2202 to determine whether the environment surrounding the user 1904 is preferred for quality sleep and/or health of the user 1904. The population level insight 2206 can be received from a data store, remote computing system, and/or cloud-based service or system. The population level insight 2206 can indicate different ranges of each ambient environmental condition that impacts user sleep quality and health. The population level insight 2206 can include ranges for the conditions 2202 including sound level, illumination, CO₂ concentration, and temperature. The population level insight 2206 can be represented in a color-coded table. For example, values represented in green are within range, values represented in yellow are deviating from an expected range/value, and values represented in red indicate substantial deviations from the expected range/value.

Sound can negatively or positively affect sleep depending on intensity, timing, and frequency of the sound. High intensity sound (e.g., >50 decibels, dB) at the beginning of sleep or during shallow sleep can disturb overall sleep while low intensity sound (up to 30 dB) may favor sleep by masking background noise. Noise that is greater than or equal to 30 dB but less than 50 dB may also disturb sleep. Therefore, it can be preferred that noise be maintained below 30 dB throughout the night in order to not disturb a user's sleep. Audio signals at 20 kilohertz (KHz) can therefore be detected and monitored by a microphone in the hub device 1902. Quality of the audio signal may be degraded depending on user (privacy related) preferences. Additional information can also be received to determine sleep quality relative to sound in the environment including but not limited to session information, raw 10s, subjective sleep quality, such as a questionnaire, and optionally signals from wearable sensors. Signals from the sensors, which may also include pressure and/or load-cell sensors, can be used to determine periods of sleep and periods of wake, which can then be correlated to sound levels in the environment. Thus, the disclosed technology can be used to determine what sound levels are affecting the user's sleep and how the environmental sounds can be masked or otherwise mitigated to improve the user's sleep.

Illumination levels that are greater than or equal to 1,000 lux (lx) can negatively impact a user's ability to fall asleep and experience quality sleep. Illumination that is greater than or equal to 500 lx and less than 1,000 lx also has a high chance of negatively impacting the user's sleep. Illumination that is greater than or equal to 10 lx and less than 500 lx may have a chance of negatively impacting the user's sleep. Illumination that is less than 10 lx is likely to have no negative impact on the user's sleep and therefore may promote improved sleep quality. Illumination levels can be measured during one or more predetermined periods of time, such as from 1 hour before entering the bed 1906 to 2 hours after entering the bed 1906. During that time period, if the illumination level is greater than 10 lx, the light can prevent the user 1904's HR from decreasing during sleep. Thus, the user 1904 may experience lower sleep quality during their sleep cycle. Light sensors in the hub device 1902 can therefore detect illumination values at one or more time intervals. For example, the light sensors can detect illumination values every 1, 5, 10, 15, 20, 25, and/or 30 minutes. One or more other time intervals can also be used. Additional information can also be received to determine sleep quality relative to light in the environment, including but not limited to session information, raw 10s (e.g., high-resolution data that reports heartrate, respiratory rate, movement level and/or bed presence at a 0.1 Hz resolution, or every 10 seconds), subjective sleep quality, and optionally signals from wearable sensor.

CO₂ concentration levels that are greater than or equal to 1,200 parts per million (ppm) can negative impact a user's sleep quality. CO₂ concentration that is greater than or equal to 800 ppm and less than 1,200 ppm may also negatively impact the user's sleep quality. Thus, CO₂ concentration that is less than 800 ppm can be preferred for the user to experience improved sleep quality. After all, CO₂ concentration that is less than 800 ppm can decrease sleep fragmentation. CO₂ sensors in the hub device 1902 can therefore detect CO₂ concentrations every 5 minutes. The decision engine 2204 can use this information and additional information to determine the user 1904's sleep quality. The additional information can include session information, raw 10s, subjective sleep quality, and optional signals from wearable sensors.

Temperature levels that are greater than 70 F or less than 60 F can negatively impact a user's sleep quality. Thus, a temperature between 60 and 70 F can promote faster sleep onset and longer, restful sleep. Temperature sensors of the hub device 1902 can detect ambient temperature values every 5 minutes. The decision engine 2204 can use this information and additional information to determine sleep quality relative to the temperature in the surrounding environment. The additional information can include but is not limited to session information, raw 10s, subjective sleep quality, and optional signals from wearable sensors. Sometimes, the user 1904's sleep quality relative to temperature can also be based on blood pressure readings that are detected from external sensors.

Still referring to the process 2200 in FIG. 22, the decision engine 2204 can be configured to determine personalized insight 2208 for the user 1904 based on the ambient environmental conditions 2202 in comparison to the population level insight 2206. The population level insight 2206 can be determined and based on age/gender and geographic location, since sleep differs depending on age and generate and geographic location affects noise level and lighting conditions in a surrounding environment. The personalized insight 2208 can also be based on analyzing historic data indicating the user 1904's sleep and overall health.

Determining the personalized insight 2208 can include graphing the conditions 2202 into a multi-dimensional matrix, where each dimension represents a different condition. The personalized insight 2208 graph can be a spider diagram, which may or may not be shown to the user 1904, but can visually indicate conditions that are associated with better sleep quality for the user 1904. Here, the dimensions (e.g., axes) include illumination, sound, temperature, and CO₂ concentration. Values can be plotted in the matrix along each of the dimensions based on the analysis of the user 1904's sleep history. The values can represent ideal illumination, sound, temperature, and CO₂ concentration levels to provide the user 1904 with improved, quality sleep.

The decision engine 2204 can also determine suggestions 2210 based on the graphed data. The suggestions 2210 can be generated to make changes to the environment that improve the user 1904's sleep quality. Example suggestions 2210 include ventilating the environment (e.g., bedroom) to lower the CO₂ concentration. Example suggestions 2210 also can include limiting sound exposure during the user 1904's sleep. One or more other suggestions 2210 can be generated by the decision engine 2204.

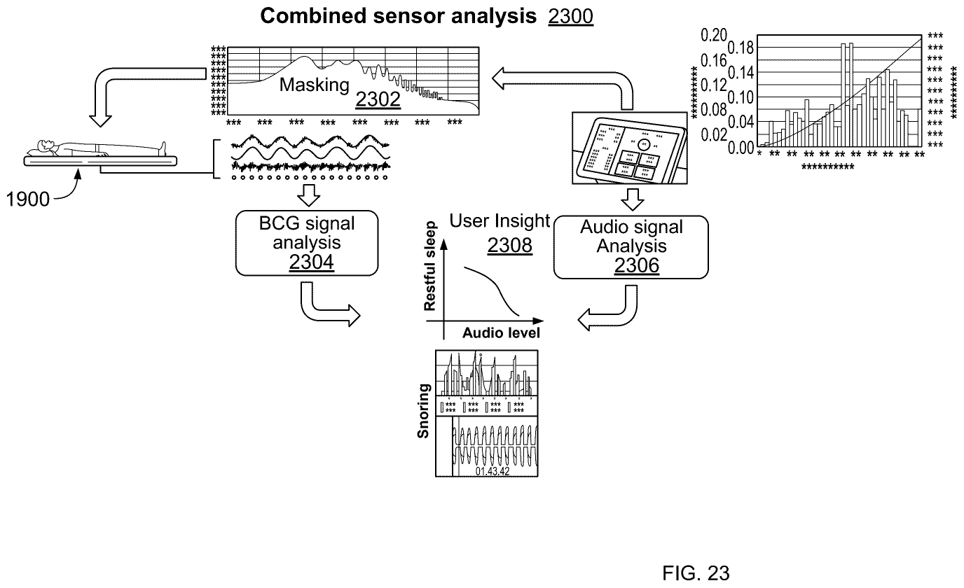

FIG. 23 is a conceptual diagram of combined sensor analysis 2300. The combined sensor analysis 2300 can be performed by the hub device 1902 and/or a remote computing system. By combining different types of sensor signals, the analysis 2300 can provide more robust user insight 2308 with regards to sleep quality, apnea, snore, and sleep stages. The analysis 2300 can also provide for improved and seamless remote health monitoring and early detection of health-related issues. The analysis 2300 can make health and wellness monitoring a part of a user's bedtime routine since clear physical markers can be identified and analyzed to proactively identify health conditions. Such physical markers can include weight, oxygen, temperature, and blood pressure. Integration of sensors via the analysis 2300 also provides for integration with third party solutions, services, and applications, which can improve accuracy in detection of health-related issues.

The analysis 2300 provides for combining data from multiple different sensors and related information from associated databases to achieve improved accuracies and more specific inferences that otherwise can be achieved via a single sensor. Therefore, the analysis 2300 is advantageous to make more accurate determinations earlier on about health-related conditions of the user.

Still referring to FIG. 23, environmental conditions and vital signs can be detected from sensors in the environment 1900. Vital signs (e.g., Ballistocardiographic, or BCG, signals) can be analyzed 2304. By analyzing the signs in 2304, user insight 2308 can be generated for the particular user. The vital signs can indicate, for example, information about snoring and/or apnea of the particular user.

Sensors of the hub device 1902 can also detect ambient environmental conditions. The hub device 1902 can perform, for example, audio signal analysis 2306 to determine whether the audio signals are related to the user snoring or apnea or environmental sound levels that may lower the user's sleep quality. The detected audio signals can also be masked in 2302 to reduce an amount of noise in the environment 1900, such as pink noise. This can be beneficial to help the user fall asleep and remain asleep. Once the audio signals and other environmental conditions are masked in 2302 and/or analyzed in 2306, such signals can be used to determine the user insight 2308.

The user insight 2308 can indicate how the user slept during a sleep cycle relative to ambient audio levels. As shown in the example user insight 2308, the user experienced less restful sleep as audio level increased in the environment 1900. Moreover during the same sleep cycle, the user also snored, which was determined based on the analysis of vital signs in 2304. By combining and analyzing values that are detected from a variety of different sensors, more robust insight can be provided about why the user may be snoring, what causes the snoring, what causes increases in ambient audio in the environment 1900, and how ambient audio can be reduced in the environment 1900 to reduce snoring or otherwise improve the user's overall sleep quality.

Audio levels in the environment 1900 can be determined using signals that are detected from microphones and similar sensors near and/or surrounding the user. The detected audio levels can be categorized into low (<30 dB), middle (30 to 50 dB), and loud (>50 dB) levels. As described herein, noise can have a negative or positive affect on sleep quality depending on timing and intensity. Loud noise while falling asleep can delay sleep onset while loud pink noise in the middle of a sleep session may protect sleep against sudden single-pitch noises. A negative effect of loud noise can be sleep fragmentation, which is an amount of time being awake between sleep onset and wakeup time. Thus, once the audio levels are categorized, the hub device 1902 can determine what environmental adjustments may be made to improve the user's sleep.

FIG. 24 is a system diagram of components that perform the techniques described herein. The hub device 1902 can include a variety of different components that can communicate with each other using one or more different connections (e.g., wired and/or wireless). For example, the hub device 1902 can include a display 2400. As described throughout this disclosure, the display 2400 can be a touchscreen that is part of the hub device 1902. The display 2400 can sometimes be a separate device in communication with the hub device 1902. For example, the display 2400 can be a touchscreen or other display screen of a mobile device. The mobile device can be a smartphone, tablet, laptop, computer, or other type of device that is used by the user.

The hub device 1902 can also include cloud-based services 2402 having data and processing engine(s), a nightstand 2404 (which can be a physical nightstand and/or a module in an application presented at the hub device 1902), and sensors 2406.

The hub device 1902 can be connected with one or more other devices, systems, and/or modules. For example, the hub device 1902 can communicate with third party application integrations 2408A-N. This communication can be via WiFi 1920 or another wireless/Internet-based connection. The third party application integrations 2408A-N can include communication between the hub device 1902 and one or more different wellness applications or services. Such wellness applications and services can be downloaded by the user from a marketplace and accessible at the hub device 1902.

The hub device 1902 can communicate with product extension modules 2410A-N. This communication can be via CAN bus 1920 or other wireless and/or wired connections. The product extension modules 2410A-N can be used to provide functionality to modify the user's bed. The modules 2410A-N can include, for example, a module to modify a climate of the bed, a module to raise or lower portions of the bed, and a module to attach or reconfigure different sensors to the bed.

The hub device 1902 can also communicate with third party sensor integrations 2412A-N. This communication can be via Bluetooth 1920 or other wireless and/or wired connections. The third party sensor integrations 2412A-N can be used to detect additional conditions, both vitals and environmental conditions. The integrations 2412A-N can include, for example, a SpO₂ sensor, a BP sensor, and/or other/miscellaneous sensors (e.g., wearable devices).

FIGS. 25A-C are system diagrams of components that perform the techniques described herein. Referring to FIG. 25A, the hub device 1902 can communicate with third party services 2500, remote computer system 1910, health providers 2502, user information data store 2504, wearable devices 2506, external sensors 2508, home automation devices 2510, and the bed 1906 via network(s) 1920.

As described herein, the third party services 2500 can offer applications that can be used by the hub device 1902 to provide additional insight into a user's health and sleep quality. The third party services 2500 can also offer applications that provide non-sleep or health related services to the user. For example, the applications can include games, weather data, stock quotes, dream diaries, virtual picture frames, alarm clocks, and other applications that can be downloaded or otherwise accessed from a marketplace where such applications are offered to the public.

The health providers 2502 can include medical and/or healthcare providers such as doctors, clinics, hospitals, and/or emergency response teams. The health providers 2502 can communicate EMRs with the hub device 1902. The health providers can also receive information from the hub device 1902, such as alerts or notifications when one or more vital signs of the user are trending outside of lower or upper ranges (e.g., refer to FIG. 21).

The wearable devices 2506 can include smart watches, heartrate monitors, bracelets, rings, wearable clothes, or other wearable devices that include sensors for tracking information about the user. The wearable devices 2506 can sense vital signs such as HR, RR, movement, etc.

The external sensors 2508 can be positioned throughout an environment surrounding the user's bed and the hub device 1902. The external sensors 2508 can detect vital signs of the user. The external sensors 2508 can also detect ambient environment conditions. In some implementations, the external sensors 2508 can be part of or otherwise integrated into the user's bed. Such sensors can be configured to detect pressure changes on a mattress, temperatures in microclimates on the mattress, snore or other audio signals, etc.

The home automation devices 2510 can be configured to control different devices, fixtures, and other elements in the environment surrounding the bed. For example, the home automation devices 2510 can be configured to raise or lower blinds, turn an HVAC system on or off, activate a security system, lock or unlock doors and windows, turn lights on or off, open or close windows, turn devices such as coffee makers and TVs on or off, etc.

The user information data store 2504 can be remote from one or more of the components described in reference to FIG. 25A. The data store 2504 can be configured to store information about each user that can be used by the hub device 1902 to more accurately make determinations about the user's sleep quality, health, and modifications that can be made to the user's sleep environment. Stored information can include user preferences for lighting, temperature, firmness of the bed, bed time, alarm clocks, and wakeup time. Stored information can also include historic sleep data, historic sleep patterns, determinations about the user's sleep quality and health, EMRs, normal environmental conditions, upper and lower ranges for monitored vital signs, and upper and lower ranges for monitored ambient environment conditions.

The hub device 1902 can include a plurality of components. As described throughout this disclosure, the hub device 1902 can include all of these components. In some implementations, the hub device 1902 can include less than all of these components. The hub device 1902 can include a microphone 2512, gas sensor 2514, pressure sensor 2516, temperature sensor 2518, humidity sensor 2520, light sensor 2522, SpO₂ sensor 2524, heartrate sensor 2526, motion sensor 2528, processor(s) 2530, memory 2532, power source 2534, display 2536, controller 2538, decision engine 2540, risk quantification engine 2542, controls engine 2544, and communication interface 2546.

The microphone 2512 can be configured to detect certain levels of audio signals in the environment, as described throughout this disclosure. For example, the microphone 2512 can detect sound pressure levels in decibels in different frequency bands including the low (<30 dB), middle (30 to 50 dB), and loud (>50 dB) noise levels in frequency bands (<2 KHz and >2 KHz but <20 KHz), which can then be correlated with sleep/wake metrics to detect a degree of influence of sound on sleep quality. A probability of waking up can depend on time since sleep onset. Sound masking techniques performed by the processor(s) 2530 of the hub device 1902 can use information such as when the user falls asleep and how long the user sleeps in order to preserve the user's sleep. In other words, audio signals that are detected by the microphone 2512 can be masked by the processor(s) 2530 in order to improve the user's sleep and keep them from waking up from ambient sounds in the environment. Detected sounds and sound masking can also be directly communicated to the user to improve the sleep environment. For example, the hub device 1902 can make suggestions for reducing ambient sound in the environment that are outputted at the display 2536. Audio signals detected by the microphone 2512 can also be used by the processor(s) 2530 of the hub device 1902 to determine whether and when the user is snoring and/or what may cause the user to snore. An extension of snore detection algorithm can enable sleep apnea detection. Detection of snore and/or apnea can be enhanced by combining and correlating signals (e.g., BCG signals) detected from other sensors, such as one or more sensors of the hub device 1902, wearable devices 2506, and/or external sensors 2508.

The gas sensor 2514 can be configured to detect air quality. The processor(s) 2530 of the hub device 1902 can use such information to determine how air quality may influence the user's sleep. The hub device 1902 can also use this information to determine what and how to improve in the user's sleep environment. For example, the gas sensor 2514 can detect CO₂ concentrations in the environment. CO₂ concentration can fluctuate based on whether windows are open or closed in the environment. Based on detected CO₂ concentrations in combination with user health-related information and current sleep information, the hub device 1902 can generate suggestions and/or automated controls to open or close windows in the environment. When ventilation occurs during sleep (e.g., thereby circulating air), users can experience a better mental state after sleep, feel more rested, and experience less next-day sleepiness. Therefore, the processor(s) 2530 of the hub device 1902 can determine whether CO₂ concentrations are within upper and lower ranges that promote a better mental state, feeling more resting, and less next-day sleepiness by using data collected by the gas sensor 2514.

The pressure sensor 2516 can be configured to detect pressure over a sleep surface. In some implementations, the pressure sensor 2516 can be part of a sensor array and/or can be integrated into a mat that covers a top surface of the user's bed. The pressure sensor 2516 can also be integrated into a base, foundation, and/or legs of a base or foundation of the user's bed. The pressure sensor 2516 can collect pressure signals indicative of movement of the user on the bed throughout their sleep cycle. The pressure sensor 2516 can also detect pressure signals that can be used to measure sleeper location on the bed, weight of the user, and biometrics data of the user, such as heartrate, breathing rate, and respiration rate. In some implementations, the pressure sensor 2516 can also be integrated into or otherwise part of a pump of the bed system. This configuration can be beneficial to reduce noise (e.g., by combining gain amplifier stage with ADC) and provide higher resolution of data acquisition than other forms of pressure sensors. In some implementations, one or more pressure sensors 2516 can be load cells that are integrated into the top surface of the mattress. Data collected by the load cells can be used by the hub device 1902 to accurate determine and monitor weight (such as for conditions like Congestive Heart Failure), biometric data (e.g., heartrate, respiratory rate), presence in the bed, user restlessness, and/or location/positioning of the user on the bed.

The temperature sensor 2518 can be configured to detect temperature measurements throughout the user's sleep environment. This can include detecting body temperature of the user as well as temperature(s) of microclimates on the surface of the bed and temperature(s) of the surrounding ambient environment. In some implementations, the temperature sensor 2518 can also be part of the bed. For example, one or more temperature sensors 2518 can be integrated into a layer of the mattress of the bed, such as on each sleeper side of the mattress. Each temperature sensor 2518 can separately detect and measure body temperature of the respective user sleeping on the bed. Temperature values detected by the temperature sensor 2518 can be incorporated into a closed loop control by the hub device 1902 in order to advance temperature control in such a way that can maintain the user at the right sleep stage. On bed thermistors, for example, can be used for accurate detection of fever conditions.

Temperature values collected by the temperature sensor 2518 can be used by the hub device 1902 to accurately detect presence of the user in the bed, detect when and if the user has a fever or other adverse health condition, and/or detect what sleep stage the user currently experiences. The hub device 1902 can also use the collected temperature values (e.g., the user's current body temperature, a microclimate temperate on the sleeper's side of the bed, etc.) to determine and, optionally, automatically implement one or more climate control changes to the bed (e.g., activating/deactivating a heating/cooling unit of the bed system).

The humidity sensor 2520 can be configured to detect humidity levels in the user's sleep environment before, during, and/or after the user's sleep cycle(s). The data collected by the humidity sensor 2520 can be transmitted and used by the hub device 1902 to determine one or more environmental modifications that can be made to improve the user's overall sleep quality and help the user maintain restful sleep.

The light sensor 2522 can be configured to detect illumination levels in the user's sleep environment before, during, and/or after the user's sleep cycle(s). During sleep, the user's heartrate should progressively decrease. However, exposure to evening bright light can limit that decrease in heartrate, thereby hindering the user's ability to fall asleep and/or experience more restful sleep. Illumination levels detected by the light sensor 2522 can be transmitted to the hub device 1902 and used by the hub device 1902 to determine suggested and/or automatic controls to adjust lighting (e.g., brightness) in the user's sleep environment. The hub device 1902′ determinations to control lighting in the sleep environment can facilitate progressive decrease in heartrate for the user, increased ability to fall asleep, and more restful sleep throughout the user's sleep cycle(s). In some implementations, the light sensor 2522 can be an ultraviolet sensor, which can sense UV radiation from the sun. UV radiation information can be transmitted to the hub device 1902 and further used by the hub device 1902 to determine brightness adjustments in the sleep environment of the user.

The SpO₂ sensor 2524 can be configured to detect oxygen saturation, photoplethysmography (PPG) peaks, pulse oximetry, heartrate, heartrate variability, and muscle oxygen saturation (SmO₂ and StO₂). PPG signals, in particular, can be used, by the hub device 1902 for example, to estimate SpO₂ for the particular user. Using the estimated SpO₂, the hub device 1902 can determine whether the user is experiencing health-related issues/conditions or is developing such issues/conditions. The SpO₂ sensor 2524 can be a pulse oximeter and/or an optical sensor. In some implementations, the SpO₂ sensor 2524 can be worn by the user, such as on their wrist, finger, ear, and/or other locations of the body.

The heartrate sensor 2526 can be configured to detect the user's heartrate while they are in bed. The hub device 1902 can use the heartrate signal and Pulse Transmit Time (PTT) to estimate the user's blood pressure. PTT can be determined as a difference between ECG peaks and PPG peaks, both of which can be measured by any one or more of the sensors described herein. The hub device 1902 can analyze the estimated blood pressure to determine whether the user is experiencing or developing any health-related issues/conditions.

The motion sensor 2528 can be configured to detect movement of the user in the sleep environment. Using the detected movement, the hub device 1902 can determine whether the user sleep walks or experiences other sleeping disorders. The hub device 1902 can also use BCG signals from the heartrate sensor 2526 or other sensors described herein to accurately detect sleep walking events and user motion throughout their sleep cycle.

In some implementations, the motion sensor 2528 can be an accelerometer (e.g., 3-axis), which can measure acceleration. The hub device 1902 may translate data from the accelerometer into biometric data about the user, based on and using BCG principles. The motion sensor 2528 can also be a gyroscope (e.g., 3-axis), which can measure angular velocity (e.g., rotations). The angular velocity data can also be translated, by the hub device 1902, into biometric data. In yet some implementations, the motion sensor 2528 can be a compass (e.g., 3-axis), which can measure directionality of the user and other objects within the surrounding environment. Directionality data can, for example, be used by the hub device 1902 to measure orientation of the user's bed.

One or more of the sensors described herein can be integrated into a sensor array. The sensor array can include a plurality of sensors that measure different data, including but not limited to an accelerometer, gyro, relative humidity, temperature, and barometric pressure sensor. The sensor array can be part of the hub device 1902. In some implementations, the sensor array, or one or more particular sensors, can be embedded into the pump of the bed system, which can be beneficial to expand sensor capabilities.

Moreover, the hub device 1902 may include one or more additional sensors and/or be in communication with one or more additional sensors that are part of the bed, worn by the user, or otherwise positioned throughout the user's sleeping environment (e.g., temperature sensors in a bedroom). As an illustrative example, an angle displacement sensor can be positioned on the top surface of the bed between the top surface of the bed and the user. The angle displacement sensor can detect biometrics data of the user, which can be transmitted to the hub device 1902 and used by the processor(s) 2530 to determine one or more biometrics of the user.

As another example, a stretch sensor can be positioned on the top surface of the bed. The stretch sensor can generate voltages when it is stretched. The voltages can be used by the hub device 1902 to measure biometrics of the user resting on the bed.

As yet another example, the hub device 1902 can include and/or be in communication with a particulate matter sensor, which can be configured to detect dust, pollution, burning, or other particulates in the surrounding sleep environment. Data collected by the particulate matter sensor can be processed by the hub device 1902 to determine one or more improvements that can be made to the environment in order to improve the user's overall sleep quality.

As another example, the hub device 1902 can include and/or be in communication with a blood pressure sensor that is separate and/or different from the other sensors described above. The blood pressure sensor can, for example, be worn by the user, such as a band around the user's wrist. The blood pressure sensor may also be part of the bed system and can be lightweight with a silent inflation feature so as to not disturb the user while they are resting on the bed. Data sensed by the blood pressure sensor can be processed by the hub device 1902 to determine blood pressure, irregular heartbeat, and/or body movement of the user. This data can also be used by the hub device 1902 to determine various types of health conditions that the user may be developing.

In yet some implementations, a singular sensor can detect multiple types of signals that are described herein. For example, the hub device 1902 can include or otherwise be in communication with an environmental sensor. The environmental sensor, similar to the gas sensor 2514, temperature sensor 2518, and humidity sensor 2520, can be configured to detect gas (e.g., volatile organic compounds, VOC), barometric pressure, ambient temperature, and relative humidity. Thus, data collected by the single environmental sensor can be used by the hub device 1902 to track air quality, determine pressure of the atmosphere, measure altitude and weather conditions, determine temperature of the atmosphere, and determine humidity in the user's sleep environment. This data can be beneficially used by the hub device 1902 to determine one or more modifications to make to the user's sleep environment to improve the user's overall sleep quality. Moreover, a singular environmental sensor can be cost effective, quiet, and have improved accuracy when implemented in the hub device 1902.

Data collected from the sensors described herein, such as ambient temperature, ambient humidity, barometric pressure, organic compounds volatiles, motion, and biometrics, can be used by the hub device 1902 to correlate sleep quality with sleeper ambient environmental conditions. Using these correlations, the hub device 1902 can generate suggestions for home thermostat settings to increase/improve sleep quality, inform users of indoor air quality concerns and methods for improving the air quality, detect potential health conditions of the user, and/or supplement chamber pressure data of the user's bed system to make more accurate determinations about the user's health conditions, sleep cycle(s), and/or sleep environment. Supplemental data can further be beneficial to increase fidelity of user motion and/or biometric data, thereby improving accuracy of in/out bed determinations, biometric coverage and resolution, system noise rejection capabilities, and sleep quality pressure delta sensitivity.

The processor(s) 2530 can be configured to perform one or more of the operations described throughout this disclosure. The memory 2532 can be configured to store (e.g., temporarily, long-term) one or more data collected by the sensors described herein and/or determined by the processor(s) 2530. In some implementations, the memory 2532 can include random access memory (RAM) of 8 Gb or 16 Gb. The memory 2532 can also include Embedded MultiMediaCard (eMMC) (e.g., Flash) memory, which can be 64 Gb, 128 Gb, 256 Gb, or 512 Gb.

The power source 2534 can be configured to plug into a power outlet to provide power to components of the hub device 1902. The power source 2534 can be an AC-DC power supply, with a power budget of approximately 10 W. The power source 2534 can include an AC-DC wall mount adapter of 5V, 2 A or 12V, 1 A. In some implementations, the power source 2534 can include a DC-DC power supply and/or regulator, which can be a 5V buck regulator. The power source 2534 can also include a power management integrated circuit (PMIC). In some implementations, the power source 2534 can include a battery. The power source 2534 can have one or more other implementations.

The display 2536 can be configured to receive user input and present output to the user. For example, the user can configure one or more ambient settings using the display 2536, such as a thermostat, automation of lights, pressure in the bed, and/or heating/cooling unit of the bed. The display 2536 can also output information to the user such as their sleep score, overall sleep quality, biometrics, and conditions in the environment that were sensed and analyzed during the user's sleep cycle by the hub device 1902. The display 2536 can, in some implementations, be a touchscreen or other interactive display. The display 2536 can be an LCD screen. Moreover, the display 2536 can be a variety of sizes and/or resolutions. For example, the display 2536 can be a 5 inch, 7 inch, or 8 inch screen. The display 2536 can also have a resolution of 800×480 or 800×1200. The display 2536 can have one or more other sizes and/or resolutions.

The controller 2538 can be configured to perform various operations described herein. For example, the controller 2538 can communicably couple to the sensors described herein. The controller 2538 can receive sensed physical phenomena from the sensors, analyze the physical phenomena to determine at least one of environmental, sleep, and health metrics of the user in the bed, and determine, based on at least one of the environmental, sleep, and health metrics of the sleeper, one or more control signals to modify the sleep environment surrounding the bed. The controller 2538 may also output, at the display 2536, at least one of the environmental, sleep, and health metrics of the user. The controller 2538 can also transmit the control signals to a second controller to engage a home automation device. The controller 2538 may also determine any one or more of the metrics described herein, based on the signals that are detected by the sensors of the hub device 1902.

The decision engine 2540 can be the same or similar to the decision engines 2106 and 2204 described in FIGS. 21-22. The decision engine 2540 can use vital signs, user information, population information, and other signals detected by the sensors described herein to determine vitals ranges of the user and whether the user is experiencing any health conditions/issues.

The risk quantification engine 2542 can provide for automatic risk quantification to determine whether the user is experiencing health issues that can and/or should be reported out. By monitoring physical phenomena during the user's sleep cycle, the risk quantification engine 2542 can more accurately track how the user's health conditions trend throughout the sleep cycle. The rick quantification engine 2542 can determine whether the user's health metrics trend outside of expected ranges for their age, gender, and other user-related information. Based on such continuous, non-invasive monitoring, the risk quantification engine 2542 can detect health-related issues early enough to get healthcare providers 2502 involved. In some implementations, for example, the risk quantification engine 2542 may transmit a notification (e.g., message, alert) to a healthcare provider 2502 of the user, notifying the healthcare provider 2502 of their condition(s).

The controls engine 2544 can be configured to determine one or more controls to adjust home automation devices and other components in the user's sleep environment using the disclosed techniques. In some implementations, the controls engine 2544 may also control one or more home automation devices and/or the bed system. For example, the controller 2538 can determine one or more operations to adjust the user's sleep environment, such as closing blinds and lowering a thermostat in the user's bedroom. These operations can be transmitted to the controls engine 2544, which the controls engine 2544 can execute.

The communication interface 2546 can be configured to provide communication between one or more of the components described herein.

In some implementations, the hub device 1902 can include any combination of one or more of the components described herein.

FIG. 25B depicts example components that can be part of the hub device 1902. The configuration depicted in FIG. 25B can be advantageous for determining health-related information about a user and how ambient environmental conditions impact the user's health. This example of the hub device 1902 can include multiple sensors for detecting various types of signals.

The hub device 1902 can include the power source 2534, the processor(s) 2530, the display 2536, Wlan/BLE radio 2548, USB stack 2550, Flash 2552, memory 2554, a light sensor 2522, a temperature, humidity, pressure, and VOC sensor 2556, an SpO₂ and HR sensor 2558, a motion sensor 2528, and a microphone 2512.

The power source 2534 can be a power supply. In some implementations, the power source 2534 can be a replaceable/rechargeable battery. The power source 2534 can be AC-DC and/or DC-DC. The display 2536 can be one or more different sizes. For example, the display 2536 can be 5 inches, 7 inches, and/or 8 inches. The display 2536 can also have one or more different resolutions, such as 800×480 and 800×1280.

The light sensor 2522 can detect illumination levels at 5 minute intervals. The light sensor 2522 may also detect illumination levels at one or more other intervals. The hub device 1902 can use such information to detect adverse changes in heartrate that may occur during sleep, which can be caused by bright light exposure throughout the user's sleep cycle. The sensor 2556 can collect gas, pressure, temperature, and/or humidity levels in the environment at 5 minute intervals (or other predetermined time intervals). The sensed values can be used by the hub device 1902 to determine one or more environmental changes that can be implemented to improve sleep quality. The sensor 2558 can detect SpO₂ and heartrate as spot measurements with a sampling rate of 500 Hz (or another predetermined sampling rate, including but not limited to 0.1 Hz to 1 KHz). In addition to oxygen saturation, SpO₂ can be used to estimate blood pressure of the user in conjunction with ECG signals, as described herein. The detected heartrate can also be used by the hub device 1902 to determine/estimate the user's blood pressure. The motion sensor 2528 can be configured to detect movement around the bed with a sample rate of 10 seconds (or another predetermined sampling rate), as described in reference to FIG. 25A. The hub device 1902 can use this information to detect sleepwalking and REM sleep disorders, for example. Finally, the microphone 2512 can detect signals indicative of snores using a 20 KHz sampling rate (or another predetermined sampling rate). Signals detected by the microphone 2512 can be used by the hub device 1902 to generate suggestions for improving noise levels in the user's sleep environment for improved sleep quality.

FIG. 25C depicts example components that can be part of the hub device 1902. The configuration depicted in FIG. 25C can be advantageous for determining sleep quality of a user and what surrounding environmental conditions impact the user's sleep quality. This example configuration of the hub device 1902 includes fewer sensors than the example configuration of the hub device 1902 in FIG. 25B. Although the example configuration in FIG. 25C may include fewer sensors, the hub device 1902 may be in communication with one or more other sensors that are part of the user's bed system, worn by the user, and/or positioned throughout the user's sleep environment. Thus, the hub device 1902 may receive supplemental data from the other sensors.

In the example configuration of FIG. 25C, the hub device 1902 can include the power source 2534, processor(s) 2530, display 2536, Wlan/BLE radio 2548, USB stack 2550, flash 2552, memory 2554, light sensor 2522, motion sensor 2528, and microphone 2512. One or more environmental sensors described throughout this disclosure can be separate from but in communication with the hub device 1902.

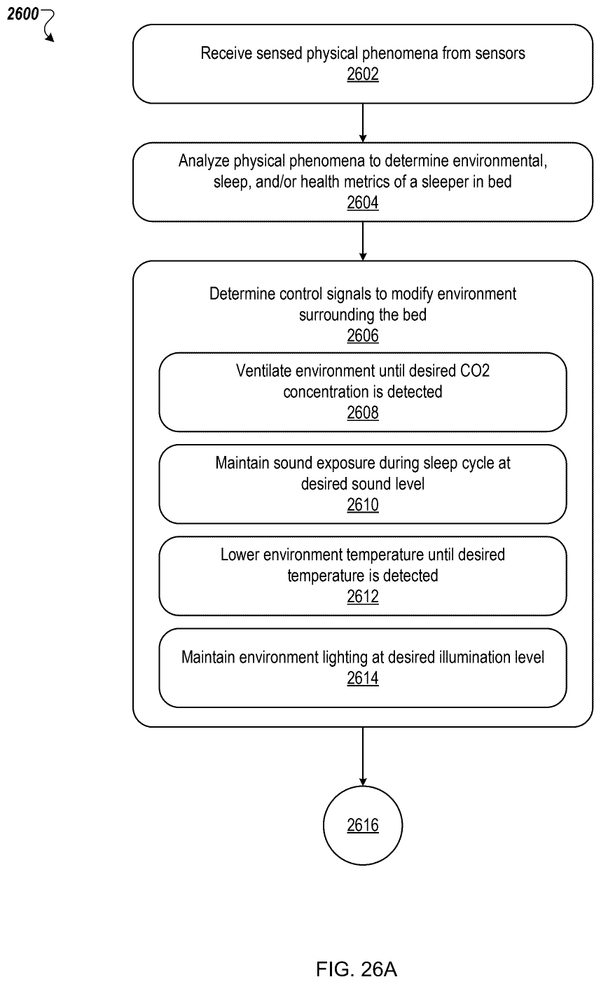

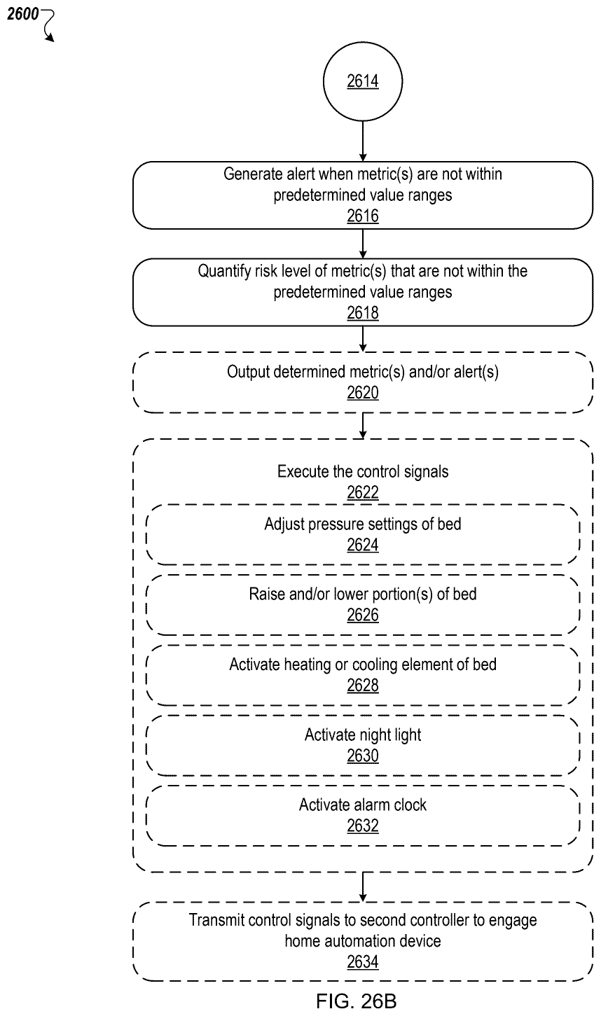

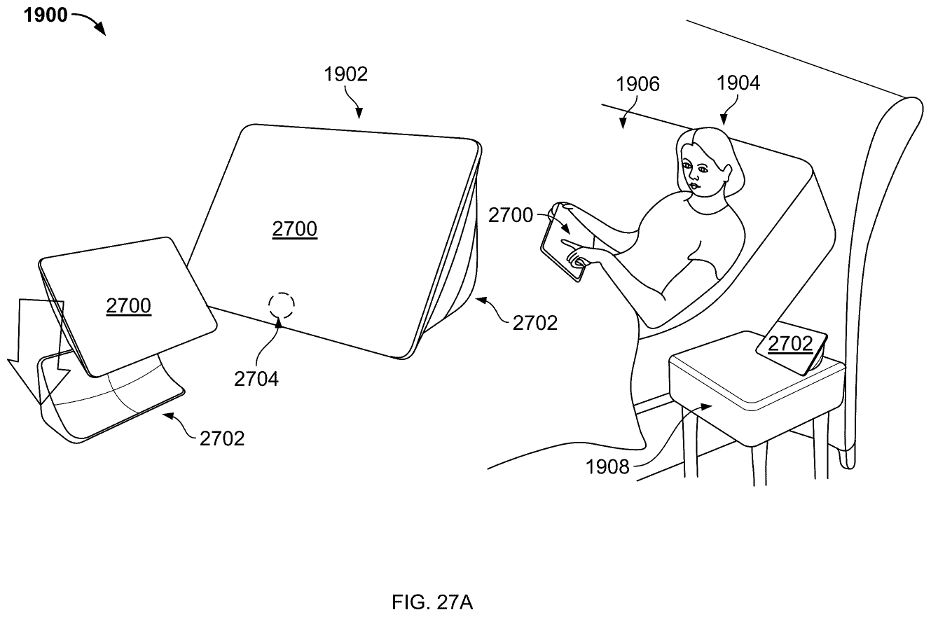

FIGS. 26A-B is a flowchart of a process 2600 for modifying an environment based on monitoring physical phenomena in the environment. The process 2600 can be performed by the hub device 1902 described throughout this disclosure. One or more blocks in the process 2600 can also be performed by one or more other computing systems and/or devices, such as the remote computer system 1910 and/or the third party services 2500. More particularly, the process 2600 can be performed by a controller of the hub device 1902 and/or the remote computer system 1910. In some implementations, the controller can be separate from sensors, a display screen (of the hub device 1902), and a user's bed. The controller can, for example, be a cloud-based system. For illustrative purposes, the process 2600 is described from the perspective of a computer system.

Referring to the process 2600 in both FIGS. 26A-B, the computer system can receive sensed physical phenomena from sensors in block 2602. As described herein, sensors can be configured to sense the physical phenomena in the environment surrounding a user's bed. The sensors can include audio, light, CO₂ concentration, temperature, humidity, motion, volatile organic compounds, electromagnetic interference, atmospheric pressure, systolic blood pressure (SBP), oxygen saturation (SPO₂), pulse, heartrate (HR), and/or radar sensors. Any of the sensors can be communicably coupled to the bed. The physical phenomena can include ambient sound, ambient light, ambient CO₂ concentration, and/or ambient temperature. The physical phenomena may also include heartrate variability (HRV), HR, respiratory rate (RR), SPO₂, SBP, and/or diastolic blood pressure (DBP).

In block 2604, the computer system can analyze the physical phenomena to determine environmental, sleep, and/or health metrics of a sleeper in a bed. The computer system can also output, at the display of the hub device 1902, at least one of the environmental, sleep, and/or health metrics of the sleeper. The computer system can also output data about the sleeper's sleep quality and/or sleep cycle at the display. Similarly, the computer system can output a health dashboard for the sleeper, weather data, stock quotes, security information associated with a home of the sleeper, lighting information in the sleep environment and/or throughout the home, and/or HVAC information in the sleep environment and/or throughout the home. The display can be a touchscreen, which can be positioned proximate to the bed in the sleep environment. In some implementations, the display can provide various functionality to the sleeper. For example, the display may receive audio input from the sleeper to control one or more home automation devices. The display may output user-selected pictures. The display may also output graphical user interfaces (GUIs) that include selectable options for the sleeper to interact with third party mobile applications that can be downloaded to and accessible via the display.

The computer system can determine health metrics of the sleeper in block 2604 using at least one of age, gender, and body mass index (BMI) of the sleeper. Such information can be retrieved from a data store (e.g., refer to FIG. 26A). Such information can be provided by the sleeper as input at the display of the hub device 1902 provided herein. For example, the computer system can determine environmental, sleep, and/or health metrics of the sleeper based on (i) sleep quality information that is provided as user input at the display of the hub device 1902 and/or (ii) physical phenomena sensed by one or more wearable devices and external sensors in communication with the computer system. The computer system may also determine whether the health metrics of the sleeper are within predetermined value ranges for each of the health metrics of the sleeper.

As another example, in block 2604, the computer system can receive audio detected by an audio sensor at 20 KHz, which can be used by the computer system to determine information about the sleeper's sleep cycle and/or sleep quality. The information about the sleep cycle and/or sleep quality can include snore and/or sleep apnea. As yet another example, the computer system can receive illumination values detected by a light sensor to determine, in combination with one or more other sensed values, changes in heartrate of the sleeper. The computer system may also determine the sleeper's sleep fragmentation using detected CO₂ concentration levels and one or more other signals from sensors described herein. Similarly, the computer system can determine how long it takes the sleeper to fall asleep and how long the sleeper experiences restful sleep based on received temperature signals in the sleep environment and one or more other sensor signals described herein. The computer system may also determine the sleeper's blood pressure and/or heartrate based on a combination of received sensor signals, including but not limited to spot measurements that are sensed by a SPO₂ sensor and/or a heartrate sensor. The computer system can also detect sleepwalking and/or REM sleep disorders based on a combination of sensor signals, including motion detected at predetermined time intervals (e.g., every 10 seconds) by a motion sensor.

The computer system can determine one or more control signals to modify an environment surrounding the bed (block 2606). The computer system can determine controls to ventilate the environment until a desired CO₂ concentration is detected in the environment (block 2608). As an illustrative example, detected ambient CO₂ concentrations can be greater than a threshold level and the controls can include ventilating the environment surrounding the bed until the CO₂ concentration in the environment is detected to be less than 800 parts per million (ppm). Ventilating the environment can include turning a fan on, opening a window, etc.

The controls can also include maintaining sound exposure during a sleep cycle of the user at a desired sound level (block 2610). For example, detected ambient sound can be greater than a threshold level and the one or more controls determined by the computer system can include maintaining the sound level at less than 30 decibels (dB) in the environment.

The controls can further include lowering an environmental temperature until a desired temperature is detected and/or maintained (block 2612). For example, detected ambient temperature can be greater than a threshold level and the controls determined by the computer system can include lowering the temperature of the environment to a value that can be greater than or equal to 60 F.° and less than or equal to 70 F.°.

The controls may also include maintaining environmental lighting at a desired illumination level (block 2614). For example, detected ambient light can be greater than a threshold level and the controls determined by the computer system can include maintaining illumination in the environment at less than 10 lux (lx). For example, during sleep, a light sensor can detect and determine whether luminous intensity is below a given threshold and then can control lighting fixtures in the environment to achieve the threshold or other desired luminous intensity. Moreover, the 10 lux threshold can be used for all users in a general population, regardless of age/gender, and/or geography.

The computer system can also generate an alert when one or more of the metrics determined in block 2605 are not within predetermined value ranges (block 2616). For example, the computer system can generate an alert based on determining that a determined health metric is not within the predetermined value range for the health metric, based on the sleeper's age, BMI, and/or other health conditions. Refer to FIG. 22 for additional discussion.

Accordingly, the computer system can quantify a risk level associated with the metric that is not within the predetermined value range in block 2618. Refer to FIG. 21 for additional discussion.

Optionally, the computer system may output the determined metric and/or the alert in block 2620. The determined metric and/or alert can be transmitted to a computing device of a healthcare provider. Therefore, the healthcare provider can be notified of a health condition/issue of the sleeper. The computer system can also output the alert at the display of the hub device 1902, thereby notifying the sleeper of their health condition/issue. In some implementations, the computer system can also generate audio output to be outputted by a speaker of the computer system, such as the hub device 1902. The audio output can indicate one or more determined metrics for the sleeper. The audio output can also include the generated alert. In yet some implementations, the audio output can include a greeting when the sleeper wakes up. The greeting can inform the sleeper of their sleep score and/or any other determined metrics.

The computer system can also optionally execute the control signals (block 2622). The control signals can be executed before and/or during any of blocks 2616-2620. Executing the control signals can optionally include adjusting pressure settings of the bed (block 2624), raising and/or lowering portions of the bed (block 2626), activating heating or cooling elements of the bed (block 2629), activating a night light (block 2630), and/or activating an alarm clock (block 2632). Therefore, the computer system can implement one or more control signals that are intended to alter the sleeper's sleep environment to improve their overall sleep quality and/or sleep experience.

Optionally, the computer system may also transmit the control signals to a second controller to engage a home automation device (block 2634). The second controller can perform any of the controls described throughout this disclosure and/or in blocks 2624-2632.

The process 2600 can be performed continuously throughout the user's sleep cycle. The process 2600 can also be performed at predetermined time intervals during the user's sleep cycle.

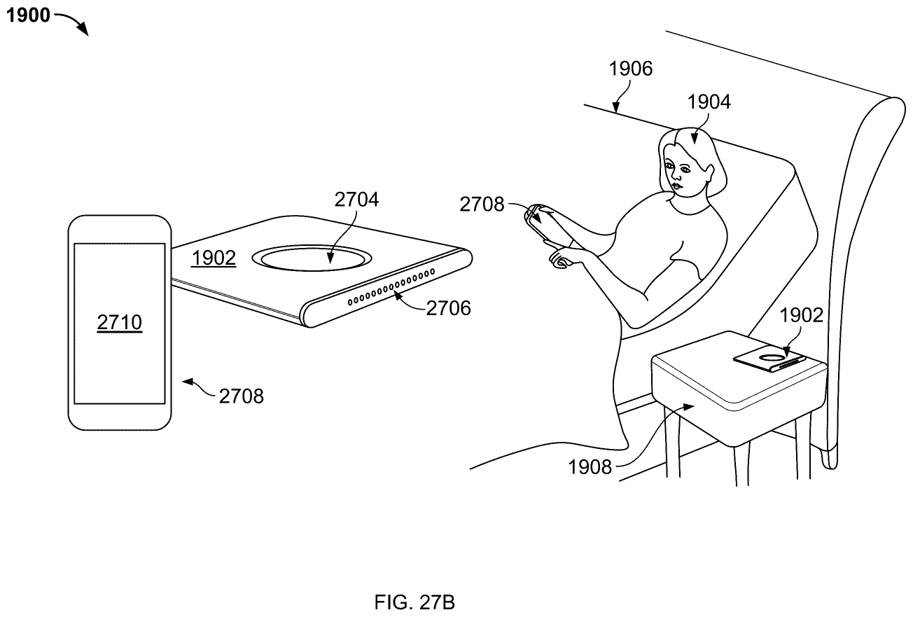

FIGS. 27A-B depict example implementations of the hub device 1902 described herein. FIG. 27A depicts the hub device 1902 in a tablet implementation. FIG. 27B depicts the hub device 1902 in a screenless implementation.

Referring to FIG. 27A, the hub device 1902 can be a tablet having an integrated interface 2700. This implementation of the hub device 1902 can provide for biometrics and health information to be determined and presented within a closed system. The hub device 1902 can include a stationary base 2702, which the hub device 1902 can rest upon at the sleeper 1904's nightstand 1908. The hub device 1902 can be ergonomic, in which the sleeper 1904 can remove the hub device 1902 from the stationary base 2702 and interact with the interface 2700 while in the bed 1906 in the bedroom 1900.

The stationary base 2702 can also include one or more of the sensors described throughout this disclosure. For example, the sensors can be integrated into a fabric or other material that encases the stationary base 2702. The hub device 1902 may also include a button 2704. The button 2704 can be on the front of the hub device 1902, such as part of the interface 2700, and can be pressed by the sleeper 1904 to measure the sleeper 1904's SPO₂ and/or body temperature. The hub device 1902 can include one or more additional features. For example, the hub device 1902 (and/or the stationary base 2702) can include one or more light sources, charging ports, and/or smart home or other home automation technology. The hub device 1902 can therefore be an integrated bedside device that can perform functions of other systems and/or devices that the sleeper 1904 may otherwise put on their nightstand 1908.

Referring to FIG. 27B, in some implementations, the hub device 1902 can be a screenless device, which communicates with other devices, such as the sleeper 1904's user device 2708, in the bedroom 1900. A screenless device can be small in size, cost less, ensure increased privacy protection, and result in fewer devices crowding a nightstand. The user device 2708 can be a mobile device, smartphone, laptop, tablet, or any other type of computing device described herein. In this implementation of the hub device 1902, biometrics and health information can be determined and presented within a mobile application that can be launched at the user device 2708. The application can provide controls for adjusting components and/or settings of the bed 1906. The application can also present any of the information determined by the hub device 1902.

The hub device 1902 can leverage existing devices such as the user device 2708 since the hub device 1902 may not have a screen in this implementation. As a result, information determined by the hub device 1902 can be presented in GUIs 2710 at the user device 2708. The sleeper 1904 can then use their device 2708 to view their sleep data as well as other metrics determined by the hub device 1902 and control components in the bedroom 1900.

Here, the hub device 1902 includes the button 2704. The hub device 1902 can also include one or more sensors 2706. The sensors 2706 may be any of the sensors described herein. The hub device 1902 can take up minimal space on the nightstand 1908. The hub device 1902 can also provide charging ports so that the sleeper 1904 can charge one or more other devices that may be placed on the nightstand or otherwise near/around the bed 1906.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

1-20. (canceled)

21. A system comprising:
an electronic hub device; and
a plurality of sensors configured to be in communication with the electronic hub device, the plurality of sensors configured to sense physical phenomena of a bed, an environment surrounding the bed, and at least one user in the bed, wherein at least one of the plurality of sensors is configured to be integrated with the electronic hub device,
wherein the electronic hub device is configured to:
receive the sensed physical phenomena from the plurality of sensors;
output information about the sensed physical phenomena to a display; and
communicate with a plurality of external devices that are configured to adjust conditions for at least one of the bed and the environment surrounding the bed.

22. The system of claim 21, further comprising a stationary base configured to receive the electronic hub device, wherein the electronic hub device is configured to be removable from the stationary base.

23. The system of claim 22, wherein at least one of the plurality of sensors is configured to be integrated with the stationary base.

24. The system of claim 21, further comprising a stationary base, wherein the electronic hub device defines a convex bottom portion and the stationary base defines a concave top portion, and wherein the concave top portion of the stationary base is configured to receive the convex bottom portion of the electronic hub device.

25. The system of claim 21, further comprising a stationary base defining a concave top portion and a flat bottom portion, the flat bottom portion of the stationary base configured to rest against an external surface, the concave top portion of the stationary base configured to receive a convex bottom portion of the electronic hub device, wherein the electronic hub device defines an integrated interface positioned opposite the convex bottom portion of the electronic hub device, the integrated interface comprising the display and an input portion configured to be interacted with by the at least one user.

26. The system of claim 21, wherein the plurality of external devices comprise (i) at least one bed controller configured to adjust conditions for the bed and (ii) at least one home automation device configured to adjust conditions for the environment surrounding the bed.

27. The system of claim 21, wherein the display is physically attached to the electronic hub device.

28. The system of claim 21, wherein the display is physically separate from the electronic hub device.

29. The system of claim 21, wherein the electronic hub device comprises a button that, when engaged, is configured to measure at least one of oxygen saturation and body temperature of the user using at least one of the plurality of sensors.

30. The system of claim 21, wherein the plurality of sensors comprise at least one of a microphone, a gas sensor, a pressure sensor, a temperature sensor, a humidity sensor, a light sensor, an oxygen saturation sensor, a heartrate sensor, a motion sensor, and a carbon dioxide sensor.

31. The system of claim 21, wherein the electronic hub device is configured to transmit health-related information of the at least one user in the bed to a healthcare provider.

32. The system of claim 21, wherein the electronic hub device is configured to generate a health score for the at least one user based on the sensed physical phenomena, wherein the health score is based on sleep data and health data associated with the at least one user.

33. The system of claim 21, wherein the electronic hub device defines a top surface, a bottom surface opposite the top surface, and a pair of opposing side surfaces between the top surface and the bottom surface, wherein the bottom surface is configured to lay flat against an external surface, wherein the top surface defines a recess that contains a button that, when engaged, is configured to initiate a function of the electronic hub device, and wherein the electronic hub device is screenless.

34. The system of claim 33, wherein the electronic hub device has a rectangular configuration when viewed from above and is sized to be held by the at least one user during use, wherein a first side surface of the pair of opposing side surfaces defines a first rounded end opposite a second rounded end, and wherein the at least one of the plurality of sensors configured to be integrated with the electronic hub device is positioned between the first and second rounded ends of the first side surface.

35. A system comprising:
a plurality of sensors configured to sense physical phenomena in an environment surrounding a bed; and
an electronic hub device configured to be communicably coupled to the plurality of sensors, wherein at least one of the plurality of sensors is configured to be integrated with the electronic hub device, the electronic hub device configured to:
receive the sensed physical phenomena from the plurality of sensors;
analyze the physical phenomena to determine at least one of environmental, sleep, and health metrics of at least one user in the bed;
output the at least one of environmental, sleep, and health metrics of the at least one user to a display; and
determine, based on the at least one environmental, sleep, and health metrics of the at least one user, one or more controls signals to modify the environment surrounding the bed.

36. The system of claim 35, wherein the display is physically attached to the electronic hub device.

37. The system of claim 35, wherein the display is physically separate from the electronic hub device, and wherein the electronic hub device is screenless.

38. The system of claim 35, wherein the electronic hub device is configured to:
transmit health-related information of the at least one user in the bed to a healthcare provider; and
generate a health score for the at least one user based on the sensed physical phenomena, the health score optionally further based on sleep data and health data associated with the at least one user.

39. A system comprising:
an electronic hub device;
a stationary base configured to receive the electronic hub device, the electronic hub device configured to be removable from the stationary base; and
a plurality of sensors configured to be in communication with the electronic hub device, the plurality of sensors configured to sense physical phenomena of a bed, an environment surrounding the bed, and at least one user in the bed, wherein at least one first sensor of the plurality of sensors is configured to be integrated with the electronic hub device, wherein at least one second sensor of the plurality of sensors is configured to be integrated with the stationary base,
wherein the electronic hub device is configured to:
receive the sensed physical phenomena from the plurality of sensors;
output information about the sensed physical phenomena to a display; and
communicate with a plurality of external devices that are configured to adjust conditions for at least one of the bed and the environment surrounding the bed.

40. The system of claim 39, wherein the plurality of sensors comprise at least one environmental sensor configured to detect air quality, barometric pressure, ambient temperature, and relative humidity associated with the environment surrounding the bed.
